(19) United States
(12) Patent Application Publication
(10) Pub. No.: US 2022/0290086 A1
(43) Pub. Date: Sep. 15, 2022

(54) METHODS AND SYSTEMS FOR CELL CULTURE

(71) Applicants: Ohio State Innovation Foundation, Columbus, OH (US); INSTITUTO TECNOLÓGICO Y DE ESTUDIOS SUPERIORES DE MONTERREY, Monterrey, Nuevo León (MX)

(72) Inventors: Carlos Fernando CEBALLOS GONZÁLEZ, Monterrey, Nuevo León (MX); Edna JOHANA BOLÍVAR MONSALVE, Monterrey, Nuevo León (MX); David DEAN, Upper Arlington, OH (US); Ciro RODRIGUEZ, Columbus, OH (US); Mario Moisés ALVAREZ, Monterrey, Nuevo León (MX); Grissel TRUJILLO DE SANTIAGO, Monterrey, Nuevo León (MX)

(21) Appl. No.: 17/635,658

(22) PCT Filed: Sep. 28, 2020
(86) PCT No.: PCT/US20/53163

§ 371 Date: Feb. 15, 2022

Related U.S. Application Data
(60) Provisional application No. 62/906,941, filed on Sep. 27, 2019.

(51) Int. Cl. C12M 1/12; C12N 5/00; C12N 5/077; C12M 1/42; C12M 1/36; B33Y 10/00; B33Y 80/00

(52) U.S. Cl. CPC C12M 25/14; C12N 5/0062; C12N 5/0669; C12M 35/04; C12M 35/02; C12M 41/48; B33Y 10/00; B33Y 80/00; C12N 2533/76; C12N 2533/54; C12N 2533/40; C12N 2501/165; C12N 2513/00

(57) ABSTRACT

Provided herein are methods for the preparation of perfusable scaffolds for cell culture. These methods can comprise providing a bioink composition and a fugitive ink composition; chaotic printing the bioink composition and the fugitive ink composition to generate a microstructured precursor comprising a plurality of lamellar structures formed from the bioink composition; curing the bioink composition to form a cured scaffold precursor; and removing the fugitive ink from the cured scaffold precursor, thereby forming the perfusable scaffold. Also provided are scaffolds prepared by these methods as well as modular bioreactors incorporating these scaffolds.

### CROSS REFERENCE TO RELATED APPLICATIONS

This application claims the benefit of U.S. Provisional Patent Application Ser. No. 62/906,941, filed Sep. 27, 2019, the disclosures of which are expressly incorporated herein by reference.

### BACKGROUND

The industrial production of well characterized, safe, and effective GMP (Good Manufacturing Practice) cells successfully supplies cell-mediated therapies, such as Leukemia therapies, based on mesenchymal stem cells (MSCs), where 10's to 100's of millions of cells are needed to treat a single patient. However, for many applications, such as personalized cancer drug-screening and regenerative medicine therapies, significantly larger quantities of cells (e.g., billions of cells) are needed. Existing cell expansion technologies require too much incubator clean room space and are too expensive to meet these needs. New cell expansion technologies are needed to support these emerging therapies and to allow them to reach the clinic.

### SUMMARY

Provided herein are method for the preparation of perfusable scaffolds for cell culture. These methods can comprise providing a bioink composition and a fugitive ink composition; chaotic printing the bioink composition and the fugitive ink composition to generate a microstructured precursor comprising a plurality of lamellar structures formed from the bioink composition; curing the bioink composition to form a cured scaffold precursor; and removing the fugitive ink from the cured scaffold precursor, thereby forming the perfusable scaffold. Importantly, these methods can rapidly and efficiently prepare microstructured scaffolds including multiple distinct layers of cells separated by controllable distances. These architectures mimic the biostructures which are involved in tissue and organ development in biological systems.

Also provided are high surface/volume, perfusable microstructured scaffolds for cell culture prepared by the chaotic printing methods described herein. In some embodiments, the perfusable scaffolds can exhibit an average striation thickness of from 10 nm to 500 μm, a surface-area-to-volume (SAV) of from 400 m⁻¹ to 5000 m⁻¹, a surface density of at least 0.05 m² cm⁻³, or any combination thereof.

Also provided are bioreactors for cell culture/expansion that comprise a plurality of the perfusable scaffolds described herein. The bioreactors can function as incubator-based systems allowing large numbers of cells to be expanded in the smallest possible space. Rather than state of the art indirectly tracked stirring systems, the bioreactor can include highly accurate sensors operatively coupled to each of the plurality of perfusable scaffolds present in the bioreactor. For example, in some embodiments, the perfusable scaffolds can be in the form of rods, fibers, or bundles of fibers. The perfusable scaffolds ran be fitted with proximal and distal collars that allow for conditions within each scaffold to be individual perfusable scaffold to be monitored in real time. For example, each collar can incorporate sensors to track environmental gases, nutrient, growth factor delivery, and waste removal. A single input plate can interface with each of the proximal and distal collars. The input plates can apply mechanical (e.g., tension, compression, and/or torsion) and/or electrical stimulation to the proximal and distal collars (and by extension scaffolds) throughout the course of cell culture.

A control system can monitor sensor readings and actuate pumps to alter, for example, media flow rate, levels of bioactive agents, etc. contacting the scaffold. Using this real-time feedback loop, the control system can provide for automated, direct chamber outlet tracking of media, flow actuation, and remote notification of the need for media additions. Unlike indirect testing in current systems, the collar sensors and associated control system can determine both when new media needs to be added, alert the user by the internet and/or wireless means (e.g., Bluetooth), and/or automatically control media flow rates of available media to ensure cell expansion rates. Unlike non-existent commercial and small scale, home-made systems that deliver non-homogenous mechanical or electrical stimulation, the collared chaotic laminar rod system will allow apply mechanical and electrical stimulation as well as allow automation of cell harvest and storage (freezing). The bioreactor can be housed in a small footprint incubator that facilitates automated and highly accurate control of environmental gases, humidity, and temperature.

Current lab-based use of non-GMP cells in standard footprint incubators can expand about 50 flasks of 1 million to 100 million cells (i.e., 5 billion cells). Whole room systems are available to expand up to 25-30 billion cells. The bioreactors described herein will accomplish this in a bench-top incubator.

### BRIEF DESCRIPTION OF DRAWINGS

The accompanying figures, which are incorporated in and constitute a part of this specification, illustrate several aspects described below.

FIGS. 1A-1F illustrate a miniaturized journal bearing (miniJB) flow system for use in chaotic printing. As shown in FIGS. 1A and 1B, an inner and an outer cylinder rotate alternately for half a cycle (n/2) in opposite directions. As shown in FIG. 1C, the miniJB system is driven by two stepper motors that are independently controlled by an Arduino platform (FIG. 1D). In this system, regular flows are obtained when concentric configurations are used (FIG. 1E), and chaotic flows can be originated at eccentric configurations (FIG. 1F). Lines show the evolution of a set of dye segments initially located (n=0) along the center-line of the external cylinder, under a protocol [2701, 8101] (n=3) in a concentric (FIG. 1E), and eccentric (FIG. 1F) configuration.

FIGS. 2A-2H illustrate the chaotic printing of microstructures in curable polymers using a miniaturized journal bearing (miniJB) flow system (both experiments and computational fluid dynamics (CFD) simulations). FIG. 2A shows the injection of a drop of ink between two eccentrically located cylinders rotating alternately in opposite directions. As shown in FIG. 2B, this results in the development of a complex microstructure after a few applications of the flow that can be preserved by curing or crosslinking. The direct simulation of JB flows, by solving the Navier-Stokes equations (using 2D CFD simulations), enables the prediction of the microstructure at any time point. Predicted microstructure evolution for an experiment in which there is a drop of fluorescent particles in a JB flow for the first three flow cycles of a mixing protocol [720°, 2160°]. FIG. 2C shows the chaotic structure experimentally produced after 4 cycles. FIG. 2D shows a 2D-CFD simulation of the dispersion of an injection of massless particles using the same mixing protocol. Scale bars: 1 mm. FIG. 2E shows the resulting microstructure is 3D in nature. A cross-section of a construct produced by chaotic mixing [270°, 810°] n=7 is (FIG. 2F) examined under a microscope, revealing a complex 3D lamellar structure in the z direction. Scale bar: 500 μm. FIG. 2G shows the evolution of the microstructure of any given JB flow recipe can be accurately predicted using 3D CFD simulations of the dispersion of a circular drop in a chaotic flow [720°, 2160°]. The resulting microstructure is depicted at different times: after n=0, 0.5, 1.0, 1.5, 1.6, 1.7, 1.8, 1.9, and 2.0 flow cycles, respectively. As shown in FIG. 2H, 3D simulations accurately reproduce structural features of the microstructure obtained experimentally. Three sections in the simulation (i, ii, and iii) are compared with images, obtained by confocal microscopy, of an experimentally obtained construct (insets ii′, and iii′). Scale bar: 1 mm

FIGS. 3A-3C illustrate the calculation of the Lyapunov exponent (Λ) for 2D JB flows. A drop composed of 10 000 points is located at time t=0 in an initial position, in a concentric (FIG. 3A) or an eccentric (FIG. 3B) JB flow. In both cases, the mixing protocol used was [270° and 810°]. The linear or exponential evolution of the filament, as stretched and folded by the regular (FIG. 3A) or chaotic (FIG. 3B) flow, is depicted for 10 full flow cycles. FIG. 3C is a plot showing the length of the filament (L/Lo), as deformed by the flow, is calculated at each quarter flow cycle and plotted. For the regular flow, the advance of the length (L/Lo) is linear, while for the chaotic flow this advance is exponential. The L value of the flow can be calculated from the slope of the straight line (ln[L/Lo] vs. time). For this particular 2D JB case, Λ=1.61 cycle⁻¹.

FIGS. 4A-4O show highly aligned micro- and nanostructures produced by chaotic printing. The growth of surface area (FIG. 4A) and the reduction in striation thickness (FIG. 4B) achievable by a chaotic printing process characterized by Λ=1.61 cycle⁻¹=2.68 min⁻¹ at 5 rpm (red symbols; by chaotic printing characterized by Λ=3.50 cycle⁻¹=2.18 min⁻¹ at 5 rpm (yellow symbols); by a state-of-the-art extrusion 3D printer (blue symbols), and by a state-of-the-art extrusion bioprinter (green symbols). As shown in FIG. 4C, fluorescent microparticles (2-15 μm in diameter) chaotically printed in PDMS using the protocol [270°, 810°] n=7 are aligned to the chaotic flow manifold to reveal a complex 3D microstructure. FIGS. 4D and 4E show close-ups of two regions within the construct. After seven flow iterations, repeated folding and stretching yields alignment at the micro scale, and (FIGS. 4E and 4F) develop a densely packed microstructure composed of parallel lamella. As shown in FIG. 4G, in some regions of the flow, after three flow iterations, this separation is on the order of tens of microns. As shown in FIG. 4H, the striation thickness distribution (STD) can be calculated from the estimation of the space between neighbor striations using image analysis techniques. FIG. 4I shows the microstructure resulting from the injection of two different types of microparticles at two different time points (n=2, green; n=3, red) and locations. Both particle sets are aligned to the flow manifold to produce a highly aligned microstructure, as revealed by experiments and (FIGS. 4J and 4K) CFD simulations. Scale bars: 500 μm. FIG. 4L shows the injection of a drop of a silver nanowire suspension into GelMA under a chaotic miniJB flow [270°, 810°] n=4. The chaotic flow rapidly stretches the pointwise injection into strings of nanowires aligned on the micro-scale. A close-up of a particular region of the construct reveals a high level of alignment and resolution. Individual nanowires rotate and align to the flow manifold. FIG. 4M shows parallel strains of carbon nanoparticles in a PDMS construct [720°, 2160°] n=4. Similarly, FIGS. 4N and 4O show gold nanoparticles chaotically printed in PDMS align to the flow manifold [720°, 2160°] n=4. FIG. 4P is a plot showing the STD (log H(S)) of the microstructure as determined by image analysis techniques. Scale bars: (FIG. 4L) 50 μm; (FIG. 4M) 20 μm; (FIG. 4N) 10 μm; (FIG. 4O) 2 μm.

FIGS. 5A-5L illustrate the chaotic bioprinting of enzymes. Microfabrication of (FIG. 5A) catalytic surfaces inspired by internal cell membranes. FIG. 5B shows a schematic of a sequential reactive enzymatic system composed of glucose oxidase and peroxidase (FIG. 5C) immobilized into nanoparticles and chaotically printed in GelMA hydrogels. As shown in FIG. 5D, glucose is added to the constructs and (FIG. 5E) the local reaction rate and extent is visualized by the change in color (colorless to red). FIG. 5F illustrates an experiment in which green nanoparticles functionalized with either enzyme were co-injected. As shown in FIGS. 5G-5H, the extent of the local reaction is denoted by the development of red fluorescence in the membrane sections. FIGS. 5I-5L show the reaction front as revealed after 5, 15, 30 and 60 seconds, respectively. Scale bars: 500 μm.

FIGS. 6A-6L illustrate the chaotic bioprinting of cells. As shown in FIG. 6A, sheets of HUVECs expressing green fluorescent protein (HUVEC-GFP) were chaotically printed in a GelMA construct and observed using optical fluorescence microscopy. FIG. 6B shows a different focal plane of the same region of the construct, revealing the three-dimensional nature of the structure. As shown in FIG. 6C, simulations show similar features within the same region. Scale bars: 500 μm. FIGS. 6D and 6E show HUVEC-GFP chaotically co-printed in GelMA containing VEGF (VEGF-GelMA). The cells first spread along a string defined by the chaotic flow manifold, and they subsequently connect across lines after 96 hours of culture, as observed by optical microscopy. Scale bars: 100 μm. FIGS. 6F-6H show different degrees of intimacy of MCF7 cancerous (red) and MCF10A healthy (green) cells co-printed in the same construct [270°, 810°] n=3. Scale bars: 500 μm. FIGS. 6I-6K show the bioprinting of GFP+RFP E. coli (green and red fluorescent bacteria) using the protocol [270°, 810°]. Different degrees of intimacy between lines of the green and red bacteria appear as time advances: (FIG. 6I) n=3: (FIG. 6J) n=4; (FIG. 6K) n=7. FIG. 6L shows the bioprinting of GFP+RFP E. coli (green and red fluorescent bacteria) using a globally chaotic protocol [720°, 2160°] at n=2. Scale bars: 500 μm.

FIGS. 7A-7D show that miniJB flow can produce either regular or chaotic flows. The geometry of the system (i.e., whether the internal cylinder is located concentrically or off-centered) and the rotation protocol determine the extent of chaos in the miniJB system. While (FIG. 7A) a concentric system will generate only regular motion, (FIG. 7B) an eccentric (off-centered) configuration and a suitable rotation protocol will produce a chaotic flow. FIG. 7C shows the simulated evolution of the microstructure developed from the stretching of different segments (shown in different colors) in a regular flow originated by a concentric system [270°, 810°] at n=3, and (FIG. 7D) in a chaotic flow originated in an eccentric system [270°, 810°] at n=3.

FIGS. 8A-8G show that different JB mixing protocols generate different microstructures. The microstructure produced by the application of different JB mixing protocols is shown, as calculated by 2D simulations. Selection of certain protocols of rotation results in the generation of either regular, partially chaotic, or globally chaotic flows within the reservoir. FIG. 8A shows a partially chaotic flow with vast islands of regular motion (zones not visited by particles) results from the application of the protocol [180°, 540°]; whereas FIG. 8B shows that a practically globally chaotic flow originates by the application of the protocol [270°, 810°]. FIG. 8C-8F show that partially chaotic flows give rise to islands of regular flow of different sizes and in different locations. FIG. 8G shows that an essentially globally chaotic flow, with a high Lyapunov (A) value, is generated by the application of the flow recipe [720°, 2160°]. All microstructures correspond to the dispersion of a circular injection of 10,000 particles, after the application of 10 flow cycles (n=10).

FIGS. 9A-9B show the exponential reduction of striation thicknesses (distances between printed lines) originated by chaotic printing. As shown in FIG. 9A, a professional printer reduces the length scales in a squared region at a linear rate. By contrast, as shown in FIG. 9B, a chaotic printer (Λ=1.61 cycle⁻¹; Λ=2.68 min⁻¹ at 5 RPM) reduces the characteristic length scales (striation thicknesses) at an exponentially fast rate.

FIGS. 10A-10F show the chaotic bioprinting of cells for tissue engineering applications. FIG. 10A shows a close-up of a string of NIH3 3T3 fibroblasts aligned by the chaotic flow within a GelMA construct. FIG. 10B shows a segment of a chaotic structure printed using an ink composed of HUVEC-GFP cells and VEGF-conjugated nanoparticles (np). Cells spread along the lines of particles after 5 days of culture. FIG. 10C shows a segment of a chaotic structure printed using an ink composed of HUVEC-GFP cells and VEGF-conjugated nanoparticles, as observed by optical microscopy after 9 days of culture. Cells have been actinDAPI stained to reveal the position of the nuclei. FIG. 10D show MCF7 cells (red) and MCF10A cells (green) co-printed in the same construct. Scale bars: 100 μm. FIG. 10E shows the printing of sacrificial gelatin sheets (rhodamine-stained) within a 3D-GelMA construct, as observed under red fluorescence and bright field illumination, (FIG. 10F) and only red fluorescence (inset). Scale bars: 100 μm.

FIGS. 11A-11B illustrate the rheology window of operation (Newtonian regime) for 3D chaotic printing using GeLMA as a matrix. FIG. 11A is a plot showing the viscosity of 10% GelMA pre-gels at different strain rates (in the low strain regime) and at different temperatures. FIG. 11B is a plot showing the viscosity of 5% GelMA pregels at different strain rates (in the low strain regime) and at different temperatures.

FIGS. 12A-12C illustrate the blinking vortex (BV)—an alternative experimental chaotic flow system. FIG. 12A shows the as-built BV flow system. Two cylinders rotate, in an alternating fashion, in the same direction. In this case, the liquid reservoir (external cylinder) does not rotate and a full flow cycle consists of a 720° rotation of the left internal cylinder and a 2160° rotation of the right internal cylinder [720°, 2160°]. This mixing recipe produces a partially chaotic flow. FIG. 12B shows a close-up of the microstructure experimentally attained with this blinking vortex flow. FIG. 12C shows a comparison of results from 2D simulations and experiments, showing the similarity between the predicted and the experimentally obtained microstructural features after 1, 2, and 3 full flow cycles.

FIGS. 13A-13G illustrate the experimental setup evaluated in Example 2. Continuous chaotic printing is based on the ability of a static mixer to create structure within a fluid. The Kenics static mixer (KSM) induces a chaotic flow by a repeated process of reorientation and splitting of fluid as it passes through the mixing elements. FIG. 13A shows a schematic representation of a KSM with two inlets on the lid. The inks are fed at a constant rate through the inlets using syringe pumps. The inks flow across the static mixer to produce a lamellar structure at the outlet. The inks are crosslinked at the exit of the KSM to stabilize the structure. Our KSM design includes a cap with two inlet ports, a straight non-mixing section that keeps the ink injections independent, a mixing section containing one or more mixing elements, and a nozzle tip. The lid can be adapted to inject several inks simultaneously. FIG. 13B shows two rotated views at 0° and 90°, of a single KSM element. FIG. 13C shows a 3D rendering of a KSM with 6 elements and schematic representation of the flow splitting action, the increase in the number of striations, and the reduction in length scales, in a KSM-printhead. The resolution, namely the number of lamellae and the distance between them (δ), can be tuned using different numbers of KSM elements. FIG. 13D is a photo showing actual continuous chaotic printing in operation. The inset FIG. 13E shows the inner lamellar structure formed at the cross-section of the printed fiber (the use of 4 KSM elements originates 16 striations). Scale bar: 250 μm. FIGS. 13F and 13G show longitudinal (FIG. 13f) and cross-sectional (FIG. 13G) microstructure of fiber obtained using different tip nozzle geometries. The images show CFD results of particle tracking experiments where two different inks containing red or green particles are coextruded through a printhead containing 4 KSM elements. The lamellar structure is preserved when the outlet diameter is reduced, from 4 mm (inner diameter of the pipe section) to 2 mm (inner diameter of the tip), through tips differing in their reduction slope.

FIGS. 14A-14F show an analysis of the reproducibility of the lamellar microstructure produced by continuous chaotic printing. FIGS. 14A and 14B show cross-sectional cuts along an alginate/graphite fiber printed using 3 KSM elements. Scale bars: 1000 μm and 500 μm for the fiber and cross-sectional cuts, respectively. As shown in FIG. 14C, the area (shaded in yellow) and perimeter (indicated in green) of graphite striations were determined using image analysis at different cross-sectional cuts. Scale bar: 500 μm. In FIG. 14D, the contours of three different cross-sectional cuts along a fiber segment are shown as different colors (green, red, and blue). The striation pattern is remarkably similar. FIGS. 14E and 14F show the results of a statistical analysis of the area (FIG. 14E, shadowed in yellow), and perimeter (FIG. 14F, indicated in green) of each of the graphite striations (indicated with numbers from 1 to 4) among 5 different cross-sectional cuts along a fiber segment chaotically printed using a printhead containing 3 KSM elements.

FIGS. 15A-15F show an evaluation of the striation profiles and mechanical properties of chaotically printed alginate/graphite fibers. FIG. 15A shows the lamellar microstructure of fibers produced with printheads containing 2, 3, 4, 5, or 6 KSM elements. The thickness of each lamella, along the red line, was determined by image analysis using Image J (shown below each cross-sectional cut). Scale bar (red): 2 mm. FIG. 15B shows how the microstructure at each cross-section was reproduced by CFD simulations, and the thickness and position of each lamella was calculated. FIGS. 15C and 15D show the Striation Thickness Distribution (STD, FIG. 15C) and cumulative STD (FIG. 15D) for constructs printed using 4, 5, 6, and 7 KSM elements. FIG. 15E shows a comparison of stress-strain curves of fibers fabricated by extrusion of pristine alginate and graphite without chaotic mixing (marked as hand-mixed) or with chaotic printing using 2, 4, or 6 KSM elements (marked as 2, 4, or 6 ke). FIG. 15F shows a comparison of the standard deviation of tensile properties (i.e. maximum stress, maximum strain, and Young's modulus for the same set of fibers; 5 fibers per treatment).

FIGS. 16A-16H show the chaotic bioprinting of bacteria. FIG. 16A shows a cross-section of a fiber where GFP- and RFP-bacteria shared an inter-material interface. The micrograph was obtained after chaotic printing at a high initial cell concentration and using 3 KSM elements. Scale bar: 500 μm. FIG. 16B shows the evolution of the concentration of living bacteria in the cross section of a fiber, initially printed with a low bacterial concentration, shown by micrographs taken at 12, 24, 36, and 48 h; Scale bar: 500 μm. FIG. 16C shows growth curves illustrating the increasing concentration of viable cells over time, as determined by standard plate culture microbiological methods. Red and green symbols indicate the evolution of red and green fluorescent bacteria, respectively. Blue symbols show the total numbers of viable cells. FIG. 16D is a plot of the natural log of bacterial populations over time. FIG. 16E shows cross-sections of alginate fibers containing fine and aligned striations of RFP-E. coli. These fibers of 1 mm thickness were produced by chaotic bioprinting using printheads containing 2 to 7 KSM elements. Scale bar: 500 μm. FIG. 16F shows a determination of the shared interface from computational simulations. FIG. 16G shows the estimation of the total amount of interface shared between regions with and without bacteria (L), normalized by the perimeter of the fiber (p); solid dots indicate approximations based on a simple geometric model, and the open dots show determinations based in image analysis of experimentally obtained micrographs. FIG. 16H is a plot of the natural log of the L/p ratio as a function of the number of elements used to print. The Lyapunov exponent (Λ) of the chaotic flow is calculated from the slope of the resulting straight line.

FIGS. 17A-17F show the bioprinting of living micro-tissues. FIGS. 17A and 17B show optical (FIG. 17A) and SEM micrographs (FIG. 17B) of the cross-sectional view of a construct in which C2C12 cells are chaotically bioprinted in an alginate/GelMA hydrogel using a 3-KSM printhead; Scale bars: 500 μm and 50 μm, respectively. FIG. 17C shows a longitudinal view of a chaotically bioprinted construct; a high cell viability is observed at the initial time, as revealed by a live/dead staining and fluorescence microscopy. Scale bar: 500 μm. Inset shows a cross-sectional cut. Scale bar: 500 μm. As shown in FIG. 17D, cells spread along the chaotically printed striations, preserving their original positions after 13 d of culture. Scale bar: 200 μm. FIG. 17E shows an optical microscopy view of a segment of fiber containing C2C12 cells 18 d after printing. Scale bar: 500 μm. FIG. 17F shows a close-up of a region stained to reveal F-actin/nuclei, showing the cell spreading and the formation of interacting cell clusters. Cell nuclei can be identified as blue dots. Actin filaments appear in red. Scale bar: 200 μm.

FIGS. 18A-18G illustrate the development of multi-scale architectures based on 3D continuous chaotic printing. FIGS. 18A-18C illustrate the 3D printing of hydrogel constructs using a KSM-printhead integrated to a commercial cartesian 3D printer. FIG. 18A is a schematic comparison of the lack (prepared using conventional extrusion techniques) and presence of internal lamellar microstructures (developed using continuous chaotic printing). FIG. 18B illustrates the printing of a long fiber arranged into a macro-scale hydrogel construct (3 cm×3 cm×4 mm). Scale bar: 5 mm. FIG. 18C shows a transverse cut of the macro-construct showing the internal microstructures. Scale bar: 1 mm. FIGS. 18D-18G show chaotic printing of fibers coupled with electrospinning. FIG. 18D is a schematic representation of the coupling between continuous chaotic printing and an electrospinning platform; an ink composed of a pristine alginate ink (4% sodium alginate in water) and an ink composed of a polyethylene oxide blend (7% polyethylene oxide in water), were coextruded through a chaotic printhead and electrospun into a nanomesh. FIG. 18E is an AFM image showing the diameter of three individual nanofibers ((1) 0.82 μm, (2) 1.05 μm, and (3) 0.437 μm) within the electrospun mesh. Scale bar: 5 μm. As shown in FIGS. 18F and 18G, photo-induced force microscopy (PiFM) reveals the lamellar nature of the nanostructure within a nanofiber (white arrows) originated using a 2-element KSM printhead (FIG. 18F) and a 3-element KSM printhead (FIG. 18G). Scale bar: 1 μm.

FIG. 19 is a schematic illustration of an example Kenics static mixer (KSM) containing four KSM elements.

FIGS. 20A-20B are a schematic illustration of an example system and method for preparing high-surface/volume, perfusable microstructures using continuous chaotic printing.

FIG. 21 shows SEM micrographs of high-surface/volume, perfusable microstructures formed using a KSM having 3, 4, 5, and 6 KSM elements.

FIG. 22 is a plot showing the distribution of channel widths in high-surface/volume, perfusable microstructures formed using a KSM having 3, 4, 5, and 6 KSM elements.

FIG. 23 schematically illustrates a perfusion bioreactor. Chaotic lamina rods can be placed in an incubator which can maintain uniform environmental gas (e.g., CO₂, dissolved 02, and H₂O-humidity) concentrations for bioreactors.

FIG. 24 schematically illustrates the parameters of a bioreactor control system. The control system can track sensor data and adjust actuators (e.g., peristaltic pumps) to keep monitored parameters in the bioreactor within desirable ranges. Mechanical stimulation can be implemented and validated for use in directing cell differentiation.

FIGS. 25A-25C schematically illustrates chaotic lamina rods in a modular bioreactor. A control system can track sensor data and adjust actuators (e.g., peristaltic pumps) to keep monitored parameters in desirable ranges. Mechanical and electrical stimulation of chaotic lamina rods can be implemented and validated for use in cell differentiation. FIG. 25A shows the full bioreactor. As shown in FIG. 25B, lamina made from “fugitive ink” can be removed as per sensor data that suggests they need to be to maintain a modeled (CFD and FSI) flow regime. FIG. 25C illustrates the increase in surface area by adding lamina from 1 to 16 l/p (length/perimeter). Using 4 rods, theoretically, the SAV is 4600 m⁻¹.

FIGS. 26A-D illustrate the concept of Surface Area To Volume (SAV) in the context of a bioreactor. Stacking fibrous sheets in the CelliGen bioreactor has an SAV of 116.7 m⁻¹. By comparison, the chaotic lamina prototype system's SAV is 710 m⁻¹.

FIG. 27 is a schematic diagram of a bioreactor described herein.

FIGS. 28A-28E shows an example system for chaotic printing in which inks are mixed and extruded through an extrusion die to produce microstructured precursors having a desired three-dimensional shape. FIGS. 28B-28E illustrate example extrusion dies.

### DETAILED DESCRIPTION

The materials, compounds, compositions, systems, and methods described herein may be understood more readily by reference to the following detailed description of specific aspects of the disclosed subject matter and the Examples and Figures included therein.

Before the present materials, compounds, compositions, and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

In this specification and in the claims which follow, reference will be made to a number of terms which shall be defined to have the following meanings:

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

“Optional” or “optionally” means that the subsequently described event or circumstance may or may not occur, and that the description includes instances where said event or circumstance occurs and instances where it does not.

Methods

Provided herein are methods for the preparation of perfusable scaffolds for cell culture. These methods can comprise providing a bioink composition and a fugitive ink composition; chaotic printing the bioink composition and the fugitive ink composition to generate a microstructured precursor comprising a plurality of lamellar structures formed from the bioink composition; curing the bioink composition to form a cured scaffold precursor; and removing the fugitive ink from the cured scaffold precursor, thereby forming the perfusable scaffold.

In some embodiments, chaotic printing can comprise a continuous process. In other embodiments, chaotic printing can comprise a batch process.

Chaotic printing of the bioink composition and the fugitive ink composition can comprise inducing a laminar flow of the bioink composition and the fugitive ink composition through a mixer. The mixer can chaotically mix the bioink composition and the fugitive ink composition, thereby forming lamellar interfaces between the bioink composition and the fugitive ink composition. In some cases, chaotic printing of the bioink composition and the fugitive ink composition can comprise coextruding the bioink composition and the fugitive ink composition through a mixer that chaotically mixes the bioink composition and the fugitive ink composition to form lamellar interfaces between the bioink composition and the fugitive ink composition.

In these embodiments, the mixer can comprise a static mixer, such as a Kenics static mixer (KSM). In some embodiments, the KSM can comprise at least two KSM elements (e.g., at least 3 KSM elements, at least 4 KSM elements, at least 5 KSM elements, at least 6 KSM elements, at least 7 KSM elements, at least 8 KSM elements, or at least 9 KSM elements). In some embodiments, the KSM can comprise 10 KSM elements or less (e.g., 9 KSM elements or less, 8 KSM elements or less, 7 KSM elements or less, 6 KSM elements or less, 5 KSM elements or less, 4 KSM elements or less, or 3 KSM elements or less).

The KSM can comprise a number of KSM elements ranging from any of the minimum values described above to any of the maximum values described above. For example, in some embodiments, the KSM can comprise from 2 to 10 KSM elements (e.g., from 2 to 7 KSM elements, or from 2 to 6 KSM elements).

In some embodiments, chaotic printing the bioink composition and the fugitive ink composition can comprise 3D printing, electrospinning, extrusion, or any combination thereof. In certain embodiments, the chaotic printing process can produce a microstructured filament or fiber. These processes can be used to form a microstructured precursor (and by extension a perfusable scaffold) having a range of 3D shapes.

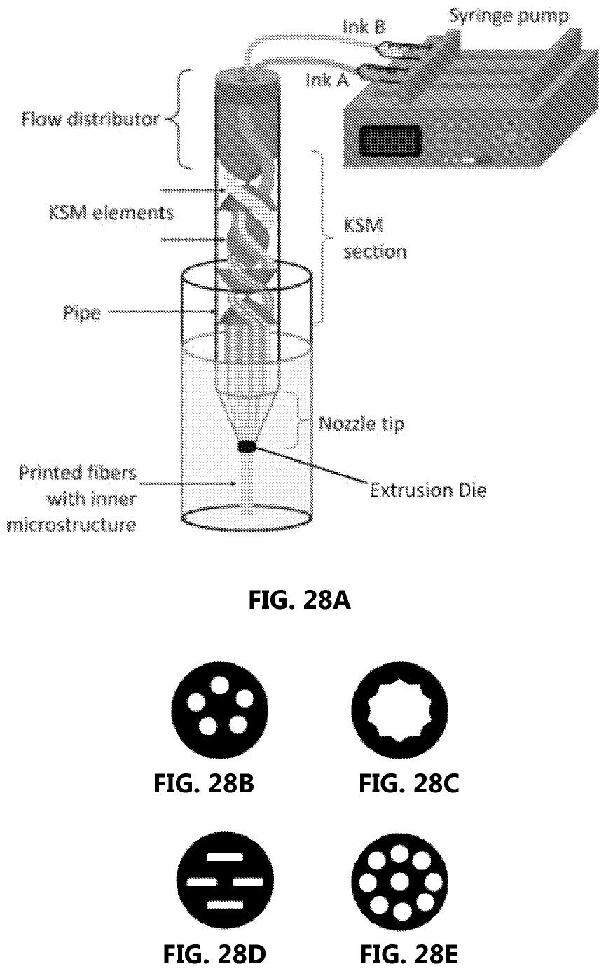

In certain examples, chaotic printing can comprise extrusion of a microstructured precursor having a variety of 3D shapes (e.g., using processes analogous to those used to produce, for example, pasta noodles of different shapes). For example, chaotic printing can comprise extrusion through a patterned extrusion die to form a microstructured precursor having a desired 3D shape and/or cross-sectional shape. A system for practicing this method is schematically illustrated in FIG. 28A. Example extrusion dies are illustrated in FIGS. 28B-28E.

In certain examples, chaotic printing can comprise of a microstructured precursor in the form of a fiber or filament. In some embodiments, these fibers or filaments can be bundled to form bundles or rods. In some embodiments, these fibers or filaments can be 3D printed or electrospun to form non-woven mats in a variety of 3D shapes.

In some embodiments, the microstructured precursor may be formed into substrate having a desired anatomical shape. For example, the microstructure precursor can be printed, spun, extruded, cast, molded, or a combination thereof to produce a precursor having the three dimensional shape of, for example, a tissue or organ. In some examples, the precursor can be formed into the shape of a patch for an organ defect (e.g., a segment of cardiac wall, vasculature, or bone), a functioning structure in an organ (e.g., a heart valve), or an entire organ (e.g., a bladder).

Once formed, the microstructured precursor (e.g., the bioink composition present in the microstructured precursor) can be cured. Suitable curing methods can be selected based on the identity of the one or more polymers present in the bioink composition. For example, in some examples, the bioink composition can comprise a polymer (e.g., alginate) which crosslinks upon exposure to a metal cation, such as Ca²⁺. In these examples, curing can comprise contacting the microstructured precursor with an aqueous solution comprising metal cations (e.g., Ca²⁺ ions). In other examples, the bioink composition can comprise one or more polymers that comprise an ethylenically unsaturated moiety. In these examples, curing can comprise exposing the microstructured precursor to UV light. In some embodiments, curing can comprise incubating the microstructured precursor (e.g., for a period of time effective for physical crosslinking of polymer

In certain embodiments, the bioink composition can exhibit a viscosity of less than 1000 cP at 23° C. prior to curing. For example, in some embodiments, the bioink composition can exhibit a viscosity of less than 500 cP, less than 250 cP, or less than 100 cP at 23° C. prior to curing. Upon curing, the bioink composition can increase in viscosity to form a matrix that exhibits a viscosity of at least 25,000 cP at 37° C. (e.g., a viscosity of from 25,000 cP to 100,000 cP at 37° C.).

In certain embodiments, the fugitive ink composition can exhibit a viscosity of less than 1000 cP at 23° C. prior to curing. For example, in some embodiments, the fugitive ink composition can exhibit a viscosity of less than 500 cP, less than 250 cP, or less than 100 cP at 23° C. prior to curing. Upon curing, the fugitive ink composition can retain a viscosity of less than 5,000 cP at 23° C. (e.g., a viscosity of less than 1000 cP, less than 500 cP, less than 250 cP, or less than 100 cP at 23° C.).

Following crosslinking, the fugitive ink can be removed from the cured scaffold precursor. The fugitive ink can be removed by any suitable method. In some embodiments, the fugitive ink can be heated and/or incubated under reduced pressure to drive off the fugitive ink. In other embodiments, the cured scaffold precursor can be immersed in an aqueous solution and/or dialyzed against an aqueous solution to remove the fugitive ink by diffusion. In other embodiments, the cured scaffold precursor can be perfused with an aqueous solution to remove the fugitive ink from within the cured scaffold precursor. Combinations of these methods can also be employed.

In some embodiments, the resulting perfusable scaffolds can exhibit an average striation thickness of at least 10 nm (e.g., at least 25 nm, at least 50 nm, at least 75 nm, at least 100 nm, at least 150 nm, at least 200 nm, at least 250 nm, at least 300 nm, at least 400 nm, at least 500 nm, at least 600 nm, at least 700 nm, at least 750 nm, at least 800 nm, at least 900 nm, at least 1 μm, at least 5 μm, at least 10 μm, at least 20 μm, at least 25 μm, at least 30 μm, at least 40 μm, at least 50 μm, at least 100 μm, at least 200 μm, at least 250 μm, at least 300 μm, or at least 400 μm). In some embodiments, the perfusable scaffolds can exhibit an average striation thickness of 500 μm or less (e.g., 400 μm or less, 300 μm or less, 250 μm or less, 200 μm or less, 100 μm or less, 50 μm or less, 40 μm or less, 30 μm or less, 25 μm or less, 20 μm or less, 10 μm or less, 5 μm or less, 1 μm or less, 900 nm or less, 800 nm or less, 750 nm or less, 700 nm or less, 600 nm or less, 500 nm or less, 400 nm or less, 300 nm or less, 250 nm or less, 200 nm or less, 150 nm or less, 100 nm or less, 75 nm or less, 50 nm or less, or 25 nm or less).

The perfusable scaffolds can exhibit an average striation thickness ranging from any of the minimum values described above to any of the maximum values described above. For example, in some embodiments, the perfusable scaffolds can exhibit an average striation thickness of from 10 nm to 500 μm (e.g., from 10 nm to 50 μm).

In other embodiments, the perfusable scaffolds can include larger striation thicknesses (e.g., striation thicknesses on the millimeter and/or centimeter length scales, such as from 1 mm to 50 cm, or from 1 mm to 10 cm).

In some embodiments, the resulting perfusable scaffolds can exhibit a surface-area-to-volume (SAV) of at least 400 m⁻¹ (e.g., at least 500 m⁻¹, at least 600 m⁻¹, at least 700 m⁻¹, at least 750 m⁻¹, at least 800 m⁻¹, at least 900 m⁻¹, at least 1000 m⁻¹, at least 1250 m⁻¹, at least 1500 m⁻¹, at least 1750 m⁻¹, at least 2000 m⁻¹, at least 2250 m⁻¹, at least 2500 m⁻¹, at least 2750 m⁻¹, at least 3000 m⁻¹, at least 3250 m⁻¹, at least 3500 m⁻¹, at least 3750 m⁻¹, at least 4000 m⁻¹, at least 4250 m⁻¹, at least 4500 m⁻¹, or at least 1750 m⁻¹). In some embodiments, the perfusable scaffolds can exhibit a surface-area-to-volume (SAV) of 5000 m⁻¹ or less (e.g., 4750 m⁻¹ or less, 4500 m⁻¹ or less, 4250 m⁻¹ or less, 4000 m⁻¹ or less, 3750 m⁻¹ or less, 3500 m⁻¹ or less, 3250 m⁻¹ or less, 3000 m⁻¹ or less, 2750 m⁻¹ or less, 2500 m⁻¹ or less, 2250 m⁻¹ or less, 2000 m⁻¹ or less, 1750 m⁻¹ or less, 1500 m⁻¹ or less, 1250 m⁻¹ or less, 1000 m⁻¹ or less, 900 m⁻¹ or less, 800 m⁻¹ or less, 750 m⁻¹ or less, 700 m⁻¹ or less, 600 m⁻¹ or less, or 500 m⁻¹ or less).

The perfusable scaffolds can exhibit a surface-area-to-volume (SAV) ranging from any of the minimum values described above to any of the maximum values described above. For example, in some embodiments, the perfusable scaffolds can exhibit a surface-area-to-volume (SAV) of from 400 m⁻¹ to 5000 m⁻¹.

In some embodiments, the resulting perfusable scaffold can exhibit a surface density of at least 0.05 m² cm⁻³ (at least 0.055 m² cm⁻³, at least 0.06 m² cm⁻³, at least 0.065 m² cm⁻³, at least 0.07 m² cm⁻³, at least 0.075 m² cm⁻³, or more),

Bioink Compositions

The bioink composition can comprise an aqueous solution comprising one or more polymers (e.g., one or more biopolymers). Following processing, the bioink will form the laminae of the microstructured scaffolds described herein. Accordingly, the one or more polymers can be selected and included in an amount effective such that the polymers form biocompatible laminae suitable to support cell culture upon curing. In some embodiments, the one or more polymers can be biodegradable.

In certain embodiments, the one or more polymers can comprise a hydrogel-forming agent. The term “hydrogel” refers to a broad class of polymeric materials, that may be natural or synthetic, which have an affinity for an aqueous medium, and may absorb large amounts of the aqueous medium, but which do not normally dissolve m the aqueous medium. Generally, a hydrogel may be formed by using at least one; or one or more types of hydrogel-forming agent, and setting or solidifying the one or more types of hydrogel-forming agent in an aqueous medium to form a three-dimensional network, wherein formation of the three-dimensional network may cause the one or more types of hydrogel-forming agent to gel so as to form the hydrogel. The term “hydrogel-forming agent”, also termed herein as “hydrogel precursor”, refers to any chemical compound that may be used to make a hydrogel. The hydrogel-forming agent may comprise a physically cross-linkable polymer, a chemically cross-linkable polymer, or mixtures thereof.

Physical crosslinking may take place via, for example, complexation, hydrogen bonding, desolvation, van der Waals interactions, or ionic bonding. In various embodiments, a hydrogel may be formed by self-assembly of one or more types of hydrogel-forming agents in an aqueous medium. The term “self-assembly” refers to a process of spontaneous organization of components of a higher order structure by reliance on the attraction of the components for each other, and without chemical bond formation between the components. For example, polymer chains may interact with each other via any one of hydrophobic forces, hydrogen bonding, Van der Waals interaction, electrostatic forces, or polymer chain entanglement, induced on the polymer chains, such that the polymer chains aggregate or coagulate in an aqueous medium to form a three-dimensional network, thereby entrapping molecules of water to form a hydrogel. Examples of physically cross-linkable polymer that may be used include, but are not limited to, gelatin, alginate, pectin, furcellaran, carageenan, chitosan, derivatives thereof, copolymers thereof; and mixtures thereof.

Chemical crosslinking refers to an interconnection between polymer chains via chemical bonding, such as, but not limited to, covalent bonding, ionic, bonding, or affinity interactions (e.g. ligand/receptor interactions, antibody/antigen interactions, etc.). Examples of chemically cross-linkable polymer that may be used include, hut are not limited to, starch, gellan gum, dextran, hyaluronic acid, polyethylene oxides), polyphosphazenes, derivatives thereof, copolymers thereof, and mixtures thereof. Other suitable polymers include polymers (gelatin, cellulose, etc.) functionalized with ethylenically unsaturated moieties (e.g., (meth)acrylate groups). Such polymers may be cross-linked in situ via polymerization of these groups. An example of such a material is gelatin methacrylate (GelMA), which is denatured collagen that is modified with photopolymerizable methacrylate (MA) groups.

Optionally, chemical cross-linking may take place in the presence of a chemical cross-linking agent. The term “chemical cross-linking agent” refers to an agent which induces chemical cross-linking. The chemical cross-linking agent may be any agent that is capable of inducing a chemical bond between adjacent polymeric chains. For example, the chemical cross-linking agent may be a chemical compound. Examples of chemical compounds that may act as cross-linking agent include, but are not limited to, 1-ethyl-3-[3-dimethylaminopropyl]carbodiimide hydrochloride (EDC), vinylamine, 2-aminoethyl methacrylate, 3-aminopropyl methacrylamide, ethylene diamine, ethylene glycol dimethacrylate, methymethacrylate, N,N′-methylene-bisacrylamide, N,N′-methylene-bis-methacrylimide, diallyitartardiamide, allyl(meth)acrylate, lower alkylene glycol di(meth)acrylate, poly lower alkylene glycol di(meth)acrylate, lower alkylene di(meth)acrylate, divinyl ether, divinyl sulfone, di- or trivinylbenzene, trimethylolpropane tri(meth)acrylate, pentaerythritol tetra(meth)acrylate, bisphenol A di(meth)acrylate, methylenebis(meth)acrylamide, triallyl phthalate, diallyl phthalate, transglutaminase, derivatives thereof or mixtures thereof. However, in some embodiments, the hydrogel-forming agents are themselves capable of chemical or physical cross-linking without using a cross-linking agent.

Besides the above-mentioned, the hydrogel-forming agents may be cross-linked using a cross-linking agent in the form of an electromagnetic wave. The cross-linking may be carried out using an electromagnetic wave, such as gamma or ultraviolet radiation, which may cause the polymeric chains to cross-link and form a three-dimensional matrix, thereby entrapping water molecules to form a hydrogel.

In some embodiments, the one or more polymers can comprise a natural polymer. A “natural polymer” refers a polymeric material that may be found in nature. In various embodiments, examples of such natural polymers include polysaccharides, glycosaminoglycans, proteins, and mixtures thereof.

Polysaccharides are carbohydrates which may be hydrolyzed to two or more monosaccharide molecules. They may contain a backbone of repeating carbohydrate i.e. sugar unit, Examples of polysaccharides include, but are not limited to, alginate, agarose, chitosan, dextran, starch, gellan gum, and mixtures thereof. Glycosaminoglycans are polysaccharides containing amino sugars as a component. Examples of glycosaminoglycans include, but are not limited to, hyaluronic acid, chondroitin sulfate, dermatin sulfate, keratin sulfate, dextran sulfate, heparin sulfate, heparin, glucuronic acid, iduronic acid, galactose, galactosamine, and glucosamine.

Peptides, which form building blocks of polypeptides and in turn proteins, generally refer to short chains of amino acids linked by peptide bonds. Typically, peptides comprise amino acid chains of about 2-100, more typically about 4-50, and most commonly about 6-20 amino acids. Polypeptides generally refer to individual straight or branched chain sequences of amino acids that are typically longer than peptides. They usually comprise at least about 20 to 1000 amino acids in length, more typically at least about 100 to 600 amino acids, and frequently at least about 200 to about 500 amino acids. Included are homopolymers of one specific amino acid, such as for example, poly-lysine. Proteins include single polypeptides as well as complexes of multiple polypeptide chains, which may be the same or different.

Proteins have diverse biological functions and can be classified into five major categories, i.e. structural proteins such as collagen, catalytic proteins such as enzymes, transport proteins such as hemoglobin, regulatory proteins such as hormones, and protective proteins such as antibodies and thrombin. Other examples of proteins include, but are not limited to, fibronectin, gelatin, fibrin, pectins, albumin, ovalbumin, and polyamino acids.

In other embodiments, the one or more polymers can comprise a synthetic polymer. Examples of suitable synthetic polymers include, for example, a polyester such as poly(propylene fumarate) (PPF), polylactic acid (PLA), polyglycolic acid (PGA), poly lactic-co-glycolide (PLGA), polycaprolactone (PCL), polydioxanone (PDS), a polyhydroxyalkanoate (PHA), a polyurethane (PU), copolymers thereof, and blends thereof. Examples of polyhydroxyalkanoates include poly-3-hydroxybutyrate (P3HB), poly-4-hydroxybutyrate (P4HB) polyhydroxyvalerate (PHV), polyhydroxyhexanoate (PHH), polyhydroxyoctanoate (PHO), copolymers thereof, and blends thereof. Other suitable biodegradable synthetic polymers include, for example, polyurethanes. In certain embodiments, the biodegradable synthetic polymer can comprise PGA.

In some embodiments, the one or more polymers can comprise alginate, agarose, or a combination thereof. In some embodiments, the one or more polymers can comprise alginate. The term “alginate” refers to any of the conventional salts of algin, which is a polysaccharide of marine algae; and which may be polymerized to form a matrix for use in drug delivery and in tissue engineering due to its biocompatibility, low toxicity, relatively low cost, and simple gelation with divalent cations such as calcium ions (Ca²⁺) and magnesium ions (Mg²⁺). Examples of alginate include sodium alginate which is water soluble, and calcium alginate which is insoluble in water. In some embodiments, the one or more polymers can comprise agarose. Agarose refers to a neutral gelling fraction of a polysaccharide complex extracted from the agarocytes of algae such as a Rhodophyceae.

In some embodiments, the one or more polymers can comprise gelatin. The term “gelatin” as used herein refers to protein substances derived from collagen. In the context of this description, “gelatin” also refers to equivalent substances such as synthetic analogues of gelatin (e.g., gelatin methacrylate (GelMA)). Generally, gelatin may be classified as alkaline gelatin, acidic gelatin, or enzymatic gelatin. Alkaline gelatin may be obtained from the treatment of collagen with a base such as sodium hydroxide or calcium hydroxide. Acidic gelatin may be Obtained from the treatment of collagen with an acid such as hydrochloric acid. Enzymatic gelatin may be obtained from the treatment of collagen with an enzyme such as hydrolase.

In certain embodiments, the bioink composition can comprise collagen, hyaluronate, fibrin, alginate, agarose, chitosan, gelatin, matrigel, glycosaminoglycans, or a combination thereof.

In some embodiments, the one or more polymers can be present in an amount of at least 0.5% by weight (e.g., at least 1.0% by weight, at least 1.5% by weight, at least 2.0% by weight, at least 2.5% by weight, at least 3% by weight, at least 4% by weight, at least 5% by weight, at least 6% by weight, at least 7% by weight, at least 8% by weight, at least 9% by weight, at least 10% by weight, at least 11% by weight, at least 12% by weight, at least 13% by weight, at least 14% by weight, at least 15% by weight, at least 16% by weight, at least 17% by weight, at least 18% by weight, or at least 19% by weight), based on the total weight of the bioink composition. In some embodiments, the one or more polymers can be present in an amount of 20% by weight or less (e.g., 19% by weight or less, 18% by weight or less, 17% by weight or less, 16% by weight or less, 15% by weight or less, 14% by weight or less, 13% by weight or less, 12% by weight or less, 11% by weight or less, 10% by weight or less, 9% by weight or less, 8% by weight or less, 7% by weight or less, 6% by weight or less, 5% by weight or less, 4% by weight or less, 3% by weight or less, 2.5% by weight or less, 2% by weight or less, 1.5% by weight or less, or 1% by weight or less), based on the total weight of the bioink composition.

The amount of the one or more polymers present in the bioink composition can be present in an amount ranging from any of the minimum values described above to any of the maximum values described above. For example, in some embodiments, the one or more polymers can be present in the bioink composition in an amount of from 0.5% to 20% by weight, based on the total weight of the bioink composition.

In certain examples, the bioink composition can comprise from 2% by weight to 10% by weight gelatin methacrylate (GelMA) and from 1% by weight to 4% by weight alginate.

In some embodiments, the bioink composition can further comprise a population of cells, one or more bioactive agents, or any combination thereof (as described in more detail below).

The bioink composition can include one or more polymers dissolved in an aqueous medium to form a solution. The terms “aqueous medium” and “aqueous solution” as used herein are used interchangeably, and refers to water or a solution based primarily on water such as phosphate buffered saline (PBS), or water containing a salt dissolved therein. The aqueous medium may also comprise or consist of a cell culture medium. The term “cell culture medium” refers to any liquid medium which enables cells proliferation. Growth media are known in the art and can be selected depending of the type of cell to be grown. For example, a growth medium for use in growing mammalian cells is Dulbecco's Modified Eagle Medium (DMEM) which can be supplemented with heat inactivated fetal bovine serum.

The bioink composition can be prepared by dissolving one or more polymers in an aqueous medium to form a solution. Agitation, for example, by stirring or sonication may be carried out to enhance the rate at which the one or more polymers dissolve in the aqueous medium. In some cases, heat energy may optionally be applied to the aqueous medium to increase the dissolution rate of the one or more polymers in the aqueous medium.

In some embodiments, the bioink can further include a population of nanoparticles, a population of microparticles, or a combination thereof. In some embodiments, the microparticles and nanoparticles can comprise polymer particles. The polymer particles can be formed from polylactides (e.g., poly(lactic acid) (PLA), poly(lactic-co-glycolic acid) (PLGA), and poly(lactic acid)-polyethyleneglycol (PLA-PEG) block copolymers), polyesters (e.g., polycaprolactone and polyhydroxyalkanoates such as poly-3-hydroxybutyrate (PHB) and poly-4-hydroxybutyrate (P4HB)), polyglycolides, polyanhydrides, poly(ester anhydrides), polyalkylene oxides (e.g., polyethylene glycols, polypropylene glycols, polybutylene glycols, and copolymers thereof), polyamines, polyurethanes, polyesteramides, polyorthoesters, polydioxanones, polyacetals, polyketals, polycarbonates, polyphosphoesters, polyoxaesters, polyorthocarbonates, polyphosphazenes, succinates, poly(malic acid), poly(amino acids), polyvinylpyrrolidone, polyethylene glycol, poly(amino acids), cellulosic polymers (e.g., cellulose and derivatives thereof, such as hydroxypropyl methyl cellulose, ethyl cellulose, methyl cellulose, sodium carboxymethyl cellulose (NaCMC), and polyhydroxycellulose), dextrans, gelatin, chitin, chitosan, alginates, hyaluronic acid, as well as copolymers (random copolymers as well as block copolymers), terpolymers and mixtures thereof.

In some embodiments, one or more bioactive agents (as discussed below) can be conjugated to the surface of the particles. In some embodiments, one or more bioactive agents can be dispersed or encapsulated within the particles. In these embodiments, the particles can provide for the controlled or sustained release of one or more bioactive agents within the laminae over time.

The bioink composition can optionally include one or more additional components, such as a photoinitiator, solvent, surfactant light attenuator, crosslinker, nutrient, or any combination thereof. In certain examples, the bioink composition can further comprise a photoinitiator to facilitate curing.

Fugitive Ink Compositions

The fugitive ink composition can comprise an aqueous solution comprising one or more polymers which can be readily removed at some point following curing. In some embodiments, the one or more polymers are not crosslinkable or otherwise curable under conditions used to cure the bioink composition. In other embodiments, the one or more polymers can be crosslinkable or otherwise curable, but form a much less robust polymer network upon curing than the cured bioink composition. For example, the one or more polymers present in the fugitive ink composition can initially crosslink or otherwise cure to form fugitive layers following curing. The fugitive layers can then be readily removed while leaving the cured bioink layers (laminae) intact. For example, in some embodiments, the fugitive layer can degrade over time, such that the fugitive layers can removed some period of time following curing. In other examples, the fugitive layers can decay or dissolve in response to a stimulus (e.g., irradiation with light, heat, contact with an enzyme, or exposure to an acid or base), allowing for removal of the fugitive layers at a desired point following curing.

Examples of suitable polymers include, but are not limited to, polylactides (e.g., poly(lactic acid) (PLA), poly(lactic-co-glycolic acid) (PLGA), and poly(lactic acid)-polyethyleneglycol (PLA-PEG) block copolymers), polyesters (e.g., polycaprolactone and polyhydroxyalkanoates such as poly-3-hydroxybutyrate (PHB) and poly-4-hydroxybutyrate (P4HB)), polyglycolides, polyanhydrides, poly(ester anhydrides), polyalkylene oxides (e.g., polyethylene glycols, polypropylene glycols, polybutylene glycols, and copolymers thereof), polyamines, polyurethanes, polyesteramides, polyorthoesters, polydioxanones, polyacetals, polyketals, polycarbonates, polyphosphoesters, polyoxaesters, polyorthocarbonates, polyphosphazenes, succinates, poly(malic acid), poly(amino acids), polyvinylpyrrolidone, polyethylene glycol, poly(amino acids), cellulosic polymers (e.g., cellulose and derivatives thereof, such as hydroxypropyl methyl cellulose, ethyl cellulose, methyl cellulose, sodium carboxymethyl cellulose (NaCMC), and polyhydroxycellulose), dextrans, gelatin, chitin, chitosan, alginates, hyaluronic acid, as well as copolymers (random copolymers as well as block copolymers), terpolymers and mixtures thereof.

In some examples, the one or more polymers can comprise a polylactide, that is, a lactic acid-based polymer that can be based solely on lactic acid or can be a copolymer based on lactic acid, glycolic acid, and/or caprolactone, which may include small amounts of other comonomers. As used herein, the term “lactic acid” includes the isomers L-lactic acid, D-lactic acid, DL-lactic acid and lactide, while the term “glycolic acid” includes glycolide. Examples include polymers selected from the group consisting of polylactide polymers, commonly referred to as PLA, poly(lactide-co-glycolide)copolymers, commonly referred to as PLGA, and poly(caprolactone-co-lactic acid) (PCL-co-LA). In some examples, the polymer may have a monomer ratio of lactic acid/glycolic acid of from about 100:0 to about 15:85, preferably from about 75:25 to about 30:70, more preferably from about 60:40 to about 40:60, and an especially useful copolymer has a monomer ratio of lactic acid/glycolic acid of about 50:50.

The poly(caprolactone-co-lactic acid) (PCL-co-LA) polymer can have a comonomer ratio of caprolactone/lactic acid of from about 10:90 to about 90:10, from about 35:65 to about 65:35; or from about 25:75 to about 75:25. In certain embodiments, the lactic acid based polymer comprises a blend of about 0% to about 90% caprolactone, about 0% to about 100% lactic acid, and about 0% to about 60% glycolic acid.

The lactic acid-based polymer can have a number average molecular weight of from about 1,000 to about 120,000 (e.g., from about 5,000 to about 50,000, or from about 8,000 to about 30,000), as determined by gel permeation chromatography (GPC). Suitable lactic acid-based polymers are available commercially. For instance, 50:50 lactic acid:glycolic acid copolymers having molecular weights of 8,000, 10,000, 30,000 and 100,000 are available from Boehringer Ingelheim (Petersburg, Va.), Medisorb Technologies International L.P. (Cincinatti, Ohio) and Birmingham Polymers, Inc. (Birmingham, Ala.) as described below.

Examples of other suitable polymers include, but are not limited to, Poly (D,L-lactide) Resomer® L104, PLA-L104, Poly (D,L-lactide-co-glycolide) 50:50 Resomer® RG502, Poly (D,L-lactide-co-glycolide) 50:50 Resomer® RG502H, Poly (D,L-lactide-co-glycolide) 50:50 Resomer® RG503, Poly (D,L-lactide-co-glycolide) 50:50 Resomer® RG506, Poly L-Lactide MW 2,000 (Resomer® L 206, Resomer® L 207, Resomer® L 209, Resomer® L 214); Poly D,L Lactide (Resomer® R 104, Resomer® R 202, Resomer® R 203, Resomer® R 206, Resomer® R 207, Resomer® R 208); Poly L-Lactide-co-D,L-lactide 90:10 (Resomer® LR 209); Poly glycolide (Resomer® G 205); Poly D,L-lactide-co-glycolide 50:50 (Resomer® RG 504 H, Resomer® RG 504, Resomer® RG 505); Poly D-L-lactide-co-glycolide 75:25 (Resomer® RG 752, Resomer® RG755, Resomer® RG 756); Poly D,L-lactide-co-glycolide 85:15 (Resomer® RG 858); Poly L-lactide-co-trimethylene carbonate 70:30 (Resomer® LT 706); Poly dioxanone (Resomer® X 210) (Boehringer Ingelheim Chemicals, Inc., Petersburg, Va.).

Additional examples include, but are not limited to, DL-lactide/glycolide 100:0 (MEDISORB® Polymer 100 DL High, MEDISORB® Polymer 100 DL Low); DL-lactide/glycolide 85/15 (MEDISORB® Polymer 8515 DL High, MEDISORB® Polymer 8515 DL Low); DL-lactide/glycolide 75/25 (MEDISORB® Polymer 7525 DL High, MEDISORB® Polymer 7525 DL Low); DL-lactide/glycolide 65/35 (MEDISORB® Polymer 6535 DL High, MEDISORB® Polymer 6535 DL Low); DL-lactide/glycolide 54/46 (MEDISORB® Polymer 5050 DL High, MEDISORB® Polymer 5050 DL Low); and DL-lactide/glycolide 54/46 (MEDISORB® Polymer 5050 DL 2A(3), MEDISORB® Polymer 5050 DL 3A(3), MEDISORB® Polymer 5050 DL 4A(3)) (Medisorb Technologies International L.P., Cincinnati, Ohio); and Poly D,L-lactide-co-glycolide 50:50; Poly D,L-lactide-co-glycolide 65:35; Poly D,L-lactide-co-glycolide 75:25; Poly D,L-lactide-co-glycolide 85:15; Poly DL-lactide; Poly L-lactide; Poly glycolide; Poly ε-caprolactone; Poly DL-lactide-co-caprolactone 25:75; and Poly DL-lactide-co-caprolactone 75:25 (Birmingham Polymers, Inc., Birmingham, Ala.).

In some examples, the one or more polymers can comprise a biodegradable, biocompatible poly(alkylene oxide) block copolymer, such as a block copolymer of polyethylene oxide and polypropylene oxide (also referred to as poloxamers). Examples of polyoxyethylene-polyoxypropylene (PEO-PPO) block copolymers include PLURONIC® F127 and F108, which are PEO-PPO block copolymers with molecular weights of 12,600 and 14,600, respectively. Each of these compounds is available from BASF of Mount Olive, N.J. PLURONIC® acid F127 in PBS.

In some examples, the one or more polymers can comprise block polymers such as polyoxyethylene-polyoxypropylene (PEO-PPO) block polymers of the general structure A-B, (A-B)ₙ, A-B-A (e.g., a poloxamer or PLURONIC®), or (A-B-A)ₙ with A being the PEO part and B being the PPO part and n being greater than 1. In other embodiments, the one or more polymers can comprise branched polymers of polyoxyethylene-polyoxypropylene (PEO-PPO) like tetra-functional poloxamines (e.g., a poloxamine or TETRONIC®). For example, the one or more polymers can comprise poloxamer 407, poloxamer 188, poloxamer 234, poloxamer 237, poloxamer 338, poloxamine 1107, poloxamine 1307, or a combination thereof.

Advantageously, poloxamers have surfactant abilities and extremely low toxicity and immunogenic responses. Thus, traces of poloxamers following removal of the fugitive ink can exhibit minimal impact on cells present in the bioink composition and/or cells subsequently seeded into the scaffold.

The average molecular weights of the poloxamers can range from about 1,000 to greater than 16,000 Daltons. Because the poloxamers are products of a sequential series of reactions, the molecular weights of the individual poloxamer molecules form a statistical distribution about the average molecular weight. In addition, commercially available poloxamers can contain substantial amounts of poly(oxyethylene) homopolymer and poly(oxyethylene)/poly(oxypropylene diblock polymers. The relative amounts of these byproducts increase as the molecular weights of the component blocks of the poloxamer increase. Depending upon the manufacturer, these byproducts may constitute from about 15 to about 50% of the total mass of the polymer.

In some embodiments, the one or more polymers can be present in an amount of at least 0.5% by weight (e.g., at least 1.0% by weight, at least 1.5% by weight, at least 2.0% by weight, at least 2.5% by weight, at least 3% by weight, at least 4% by weight, at least 5% by weight, at least 6% by weight, at least 7% by weight, at least 8% by weight, at least 9% by weight, at least 10% by weight, at least 11% by weight, at least 12% by weight, at least 13% by weight, at least 14% by weight, at least 15% by weight, at least 16% by weight, at least 17% by weight, at least 18% by weight, or at least 19% by weight), based on the total weight of the fugitive ink composition. In some embodiments, the one or more polymers can be present in an amount of 20% by weight or less (e.g., 19% by weight or less, 18% by weight or less, 17% by weight or less, 16% by weight or less, 15% by weight or less, 14% by weight or less, 13% by weight or less, 12% by weight or less, 11% by weight or less, 10% by weight or less, 9% by weight or less, 8% by weight or less, 7% by weight or less, 6% by weight or less, 5% by weight or less, 4% by weight or less, 3% by weight or less, 2.5% by weight or less, 2% by weight or less, 1.5% by weight or less, or 1% by weight or less), based on the total weight of the fugitive ink composition.

The amount of the one or more polymers present in the fugitive ink composition can be present in an amount ranging from any of the minimum values described above to any of the maximum values described above. For example, in some embodiments, the one or more polymers can be present in the fugitive ink composition in an amount of from 0.5% to 20% by weight, based on the total weight of the fugitive ink composition.

The fugitive ink composition can include one or more polymers dissolved in an aqueous medium to form a solution. The terms “aqueous medium” and “aqueous solution” as used herein are used interchangeably, and refers to water or a solution based primarily on water such as phosphate buffered saline (PBS), or water containing a salt dissolved therein. The aqueous medium may also comprise or consist of a cell culture medium. The term “cell culture medium” refers to any liquid medium which enables cells proliferation. Growth media are known in the art and can be selected depending of the type of cell to be grown. For example, a growth medium for use in growing mammalian cells is Dulbecco's Modified Eagle Medium (DMEM) which can be supplemented with heat inactivated fetal bovine serum.

The fugitive ink composition can be prepared by dissolving one or more polymers in an aqueous medium to form a solution. Agitation, for example, by stirring or sonication may be carried out to enhance the rate at which the one or more polymers dissolve in the aqueous medium. In some cases, heat energy may optionally be applied to the aqueous medium to increase the dissolution rate of the one or more polymers in the aqueous medium.

Cells

Methods can further comprise incorporating a population of cells within the perfusable scaffolds described herein. In some embodiments, methods can further comprise dispersing a population of cells in the bioink composition prior to chaotic printing. As a result, the population of cells can be printed within the perfusable scaffold. Such methods can provide for careful control of cell density and position throughout the perfusable scaffold. For example, by including a population of cells within the bioink composition, scaffolds can be printed including adjacent layers of different types of cells, layers as thin as one cell thick, and/or layers spaced apart from adjacent layers by controllable distances. Such scaffolds mimic environments observed, for example, within an embryo. As such, the scaffolds can provide in improved environment in which to control, for example, cellular differentiation. In certain embodiments, two or more distinct populations of cells (e.g., two different types of cells can be printed within the perfusable scaffold. In other embodiments, the perfusable scaffold can be seeded with a population of cells following printing (e.g., by profusion with a fluid containing a population of cells dispersed therein).

The population of cells can include any desired population of viable cells. The viable cells may include any mammalian cell type selected from cells that make up the mammalian body, including germ cells, somatic cells, and stem cells. Depending on the type of cell, cells that make up the mammalian body can be derived from one of the three primary germ cell layers in the very early embryo: endoderm, ectoderm or mesoderm. The term “germ cells” refers to any line of cells that give rise to gametes (eggs and sperm). The term “somatic cells” refers to any biological cells forming the body of a multicellular organism any cell other than a gamete, germ cell, gametocyte or undifferentiated stem cell.

For example, in mammals, somatic cells make up all the internal organs, skin, bones, blood and connective tissue. As such, a cell may include any somatic cell isolated from mammalian tissue, including organs, skin, bones, blood and connective tissue (i.e., stromal cells). Examples of somatic cells include fibroblasts, chondrocytes, osteoblasts, tendon cells, mast cells, wandering cells, immune cells, pericytes, inflammatory cells, endothelial cells, myocytes (cardiac, skeletal and smooth muscle cells), adipocytes (i.e., lipocytes or fat cells), parenchyma cells (neurons and glial cells, nephron cells, hepatocytes, pancreatic cells, lung parenchyma cells) and non-parenchymal cells (e.g., sinusoidal hepatic endothelial cells, Kupffer cells and hepatic stellate cells). The term “stem cells” refers to cells that have the ability to divide for indefinite periods and to give rise to virtually all of the tissues of the mammalian body, including specialized cells. The stem cells include pluripotent cells, which upon undergoing further specialization become multipotent progenitor cells that can give rise to functional or somatic cells. Examples of stem and progenitor cells include hematopoietic stem cells (adult stem cells; i.e., hemocytoblasts) from the bone marrow that give rise to red blood cells, white blood cells, and platelets; mesenchymal stem cells (adult stem cells) from the bone marrow that give rise to stromal cells, fat cells, and types of bone cells; epithelial stem cells (progenitor cells) that give rise to the various types of skin cells; neural stem cells and neural progenitor cells that give rise to neuronal and glial cells; and muscle satellite cells (progenitor cells) that contribute to differentiated muscle tissue.

In some examples, the cells can comprise pluripotent stem cells, multipotent stem cells, progenitor cells, terminally differentiated cells, endothelial cells, endothelial progenitor cells, immortalized cell lines, primary cells, or any combination thereof.

When added to the bioink composition prior to chaotic printing, the concentration of cells may vary depending on the composition and quantity of the bioink composition. In embodiments, the concentration of cells in the bioink composition may be in the range from about 1×10³ cells ml⁻¹ to about 1×10¹⁰ cells ml⁻¹ of bioink composition, such as about 1×10³ cells ml⁻¹ to about 1×10⁷ cells ml⁻¹, about 1×10⁵ cells ml⁻¹ to about 1×10⁷ cells ml⁻¹, about 1×10⁵ cells ml⁻¹ to about 1×10¹⁰ cells ml⁻¹, about 1×10⁷ cells ml⁻¹ to about 1×10¹⁰ cells ml⁻¹, about 1×10⁵ cells ml⁻¹, about 1×10⁶ cells ml⁻¹, about 1×10⁷ cells ml⁻¹, or about 1×10⁸ cells ml⁻¹.

Bioactive Agents

In some embodiments, the bioink composition can further include one or more bioactive agents. These bioactive agents can ultimately be incorporated into the laminae of the scaffolds therein. In some embodiments, the bioactive agents can be dissolved or dispersed in the bioink composition. In some embodiments, the bioactive agents can be bioconjugated to one or more polymers present in the bioink composition. In other embodiments, the perfusable scaffold can be treated with one or more bioactive agents following synthesis (e.g., by perfusing the scaffold with a solution or suspension comprising one or more bioactive agents). Such an approach may also be used to generate gradients of cues within the scaffold. Cells respond to gradients of fixed and diffusible chemical cues during development, wound healing and inflammatory responses that can direct cell migration, proliferation and differentiation.

As used herein, “bioactive agents” refers to any chemical substances that have an effect in a biological system, whether such system is in vitro, in vivo, or in situ. Examples of classes of bioactive agents include, but are not limited to growth factors, cytokines, antiseptics, antibiotics, anti-inflammatory agents, chemotherapeutic agents, clotting agents, metabolites, chemoattractants, hormones, steroids, morphogens, growth inhibitors, other drugs, or cell attachment molecules.

The term “growth factors” refers to factors affecting the function of cells such as osteogenic cells, fibroblasts, neural cells, endothelial cells, epithelial cells, keratinocytes, chondrocytes, myocytes, cells from joint ligaments, and cells from the nucleus pulposis. Examples of growth factors include platelet derived growth factors (PDGF), the transforming growth factors (TGF-beta), insulin-like growth factors (IGFs), fibroblast growth factors (FGFs), VEGF, EGF, and the bone morphogenetic proteins (BMPs).

The term “cytokines” refers to peptide protein mediators that are produced by immune cells to modulate cellular functions. Examples of cytokines include, but are not limited to, interleukin-1β (IL-1β), interleukin-6 (IL-6), and tumor necrosis factor-α (TNFα).

The term “antiseptics” refers to a chemical agent that inhibits growth of disease-carrying microorganisms. Examples of antiseptics include peroxides, C6-C14 alkyl carboxylic acids and alkyl ester carboxylic adds, antimicrobial natural oils, antimicrobial metals and metal salts such as silver, copper, zinc and their salts.

The term “antibiotic” includes bactericidal, fungicidal, and infection-preventing drugs which are substantially water-soluble such as, for example, gentamicin, vancomycin, penicillin and cephalosporins. An antibiotic can be added, for example, for selection of the cells or to prevent bacterial growth.

The term “anti-inflammatory agent” refers to any agent possessing the ability to reduce or eliminate cerebral edema (fluid accumulation) or cerebral ischemia, and can include examples such as free radical scavengers and antioxidants, non steroidal anti-inflammatory drugs, steroidal anti-inflammatory agents, stress proteins, or NMDA antagonists.

The term “chemotherapeutic agents” refer to any natural or synthetic molecules that are effective against one or more forms of cancer, and may include molecules that are cytotoxic (anti-cancer agent), simulate the immune system (immune stimulator), or molecules that modulate or inhibit angiogenesis. Examples of chemotherapeutic agents include alkylating agents, antimetabolites, taxanesm, cytotoxics, and cytoprotectant adjuvants.

The term “clotting agent” refers to refers to any molecule or compound that promotes the clotting of blood. Examples of clotting agents include a thrombin agent, which is commonly used as a topical treatment by vascular surgeons to stop surface bleeding after a large surface incision is made in the body, heparin, warfarin, and coumarin derivatives.

The term “metabolite” refers to an intermediate or a product derived from enzymatic conversion of a substrate administered to a subject, the conversion occurring as part of a metabolic process of the subject. Examples of metabolite include glucose, carbohydrates, amino acids and lipids.

The term “chemoattractants” refers to a substance that elicits accumulation of cells, such as chemokines, monocyte chemoattractant protein-1, and galectin-3.

The term “hormone” refers to trace substances produced by various endocrine glands which serve as chemical messengers carried by the blood to various target organs, where they regulate a variety of physiological and metabolic activities in vertebrates. Examples of hormones include steroidal estrogens, progestins, androgens, and the progestational hormone progesterone. Steroids may also be classified as lipids. Naturally occurring steroids are hormones that are important regulators of animal development and metabolism at very low concentrations. Examples of steroids include cholesterol, cortisone, and derivatives of estrogens and progesterones.

The term “cell attachment molecules” as used herein includes, but is not limited to, fibronectin, vitronectin, collagen type I, osteopontin, bone sialoprotein thrombospondin, and fibrinogen. Such molecules are important in the attachment of anchorage-dependent cells.

Bioreactors

Also provided are bioreactors for cell culture/expansion. The bioreactors can be configured to maintain and incubate a plurality of the perfusable scaffolds described herein under conditions suitable for growth of the cells. That is, the bioreactor can house the perfusable scaffolds described herein under adequate environmental conditions to permit a population of cells present therein to survive, proliferate, differentiate and/or express certain products. “Cell growth” means that the cells survive and preferably, though not exclusively, divide and multiply. In some embodiments, the perfusable scaffolds, the bioreactor, or a combination thereof may be adapted to induce tissue generation.

The bioreactors described herein can function as incubator-based systems allowing large numbers of cells to be expanded in the smallest possible space. Rather than state of the art indirectly tracked stirring systems, the bioreactor can include highly accurate sensors operatively coupled to each of the plurality of perfusable scaffolds present in the bioreactor. For example, in some embodiments, the perfusable scaffolds can be in the form of rods, fibers, or bundles of fibers. The perfusable scaffolds can be fitted with proximal and distal collars that allow for conditions within each scaffold to be individual perfusable scaffold to be monitored in real time. For example, each collar can incorporate sensors to track environmental gases, nutrient, growth factor delivery, and waste removal. A single input plate can interface with each of the proximal and distal collars. The input plates can apply mechanical (e.g., tension, compression, and/or torsion) and/or electrical stimulation to the proximal and distal collars (and by extension scaffolds) throughout the course of cell culture.

A control system can monitor sensor readings and actuate pumps to alter, for example, media flow rate, levels of bioactive agents, etc. contacting the scaffold. Using this real-time feedback loop, the control system can provide for automated, direct chamber outlet tracking of media, flow actuation, and remote notification of the need for media additions. Unlike indirect testing in current systems, the collar sensors and associated control system can determine both when new media needs to be added, alert the user by the internet and/or wireless means (e.g., Bluetooth), and/or automatically control media flow rates of available media to ensure cell expansion rates. Unlike non-existent commercial and small scale, home-made systems that deliver non-homogenous mechanical or electrical stimulation, the collared chaotic laminar rod system will allow apply mechanical and electrical stimulation as well as allow automation of cell harvest and storage (freezing). The bioreactor can be housed in a small footprint incubator that facilitates automated and highly accurate control of environmental gases, humidity, and temperature.

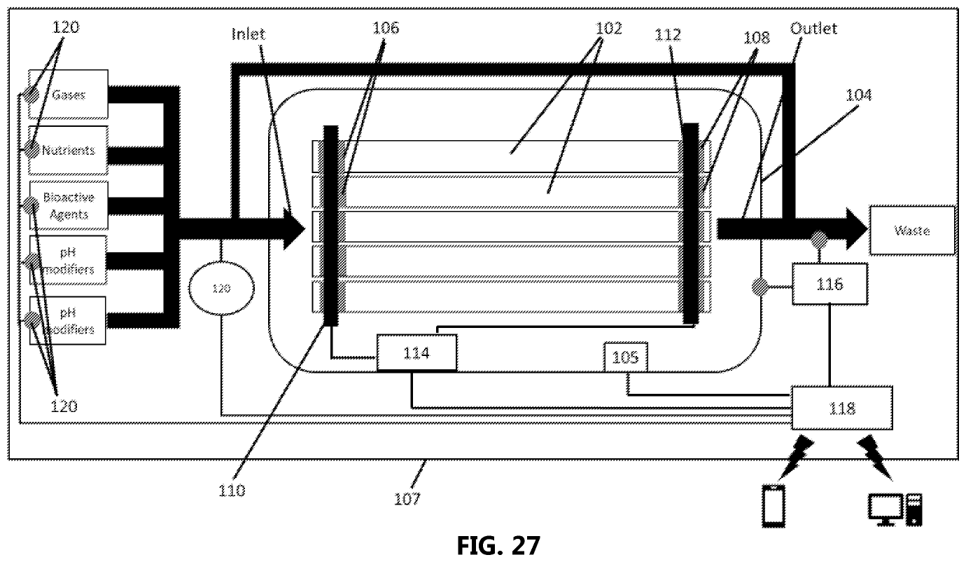

Referring to FIG. 27, in one embodiment the bioreactor can comprise a plurality of perfusable scaffolds (102), each prepared by the method of any of claims 1-26. Each of the plurality of the perfusable scaffolds is in the form of a rod, fiber, or bundle of fibers. A housing (104) can enclose the plurality of perfusable scaffolds. Heating and cooling elements (105) can be positioned within the housing to allow for thermostatic control of the interior of the housing (e.g., to maintain physiological temperature within the housing). Likewise, sensors (116) can be positioned to monitor temperature within the housing.

Each of the plurality of perfusable scaffolds (102) is operatively coupled to a proximal collar (106) and a distal collar (108). A first single input plate (110) can be operatively coupled to each of the proximal collars (106), and a second single input plate (112) can be operatively coupled to each of the distal collars (108). The first single input plate (110) and the second single input plate (112) can be operatively coupled to one or more actuators (114) that can apply mechanical stimulation to the plurality of perfusable scaffolds, electrical stimulation to the plurality of perfusable scaffolds, or a combination thereof. Sensors (116) can also be operatively connected to the outlet to monitor the composition of fluid flowing from the outlet (e.g., environmental gases, nutrients, growth factor delivery, concentration of biomarkers, concentration of bioactive agents, pH and waste removal) in real time.

In some embodiments, some or all of the system can be positioned within an incubator (107). A control system (118) can monitor sensor readings and actuate pumps (120) to alter, for example, media flow rate, levels of bioactive agents, etc. contacting the scaffold. Using this real-time feedback loop, the control system (118) can provide for automated, direct chamber outlet tracking of media, flow actuation, and remote notification of the need for media additions. Unlike indirect testing in current systems, the collar sensors and associated control system can determine both when new media needs to be added, alert the user by the internet and/or wireless means (e.g., Bluetooth). These aspects of the bioreactor can operate as a pH monitoring and control system, a temperature monitoring and control system, an O₂ monitoring and control system, a CO₂ monitoring and control system, a glucose monitoring and control system, a lactate monitoring and control system, a fluid flow monitoring and control system, or any combination thereof (depending on which components are individually sensed by sensors in the bioreactor, present in reservoirs, and coupled to pumps operated by the control system).

Current lab-based use of non-GMP cells in standard footprint incubators can expand about 50 flasks of 1 million to 100 million cells (i.e., 5 billion cells). Whole room systems are available to expand up to 25-30 billion cells. The bioreactors described herein will accomplish this in a bench-top incubator. For comparison, example densities of different bioreactor systems are compared in the table below.

Cells per milliliter: Conventional bioreactors vs. bioreactors described herein.

| Bioreactor | cells/mL* |
|---|---|
| In a T-175 cell culture bottle | 7.37 × 10⁵ |
| In a Petri Dish (9 cm diameter) | 2.72 × 10⁶ |
| Hollow-fiber bioreactors | 3.17 × 10⁶ |
| Spinner with Microcarriers | 7.85 × 10⁶ |
| In a Fedbatch bioreactor (suspension culture) | 3.85 × 10⁷ |
| Bioreactors described herein | 1.25 × 10⁸ |

*Calculated assuming a cell density of 2.72 × 10⁶ cell/cm²

Moreover, scaffold modularity can improve expansion speed, require less handling or space for freezing, and the use of standard incubators with direct sensing/controls will reduce cost at least 10×.

By way of non-limiting illustration, examples of certain embodiments of the present disclosure are given below.

### EXAMPLES

### Example 1. Chaotic Printing: Using Chaos to Fabricate Densely Packed Micro- and Nanostructures at High Resolution and Speed

Introduction

Complex microstructures are one of the signatures of nature. In natural phenomena and in many relevant applications, the microstructure of a material defines its macroscale functionality. The local rates of reaction, heat and mass transfer, ion and electron fluxes, material strength, and other important properties, depend on the extent of the interface between materials. Therefore, controlling the amount of surface area between materials is a key design criterion for the development of artificial tissues, biomimetic structures, reinforced constructs, energy harvesting systems, permeable membranes, sensors and supercapacitors, micro- or nano-sheets and super-catalytic surfaces. State-of-the art three-dimensional (3D) printing (and bioprinting) technologies have shown their potential to create complex architectures for a wide range of applications, including electronics, electronics, micro-fluidics, biomedicine, and art, and their resolution has reached the order of tens of microns. However, these technologies fail to fabricate high resolution multi-layered microstructures efficiently. Most 3D printing platforms rely on the use of printing heads (mini- or micro extruders of polymers) that move in the X, Y, and Z space to deposit streams of a material at a constant rate in a layer-by-layer fashion. Therefore, they are only capable of creating fine microstructures at a linear rate. Bioprinting technologies are even more limited in their resolution (hundreds to tens of microns). Moreover, the precise control of the position of each printed particle is another limitation of the current bioprinting technologies.

In this example, we describe a process referred to as chaotic printing: the use of chaotic flows to fabricate complex, aligned, and high-resolution 3D microstructures in a controllable, predictable, and reproducible manner in solidifiable materials. We exploit the inherent and well-studied property of chaotic processes of producing the structure at an exponentially fast rate, and use it, for the first time, for micro-fabrication purposes.

Using Chaos to Fabricate Fine Microstructures

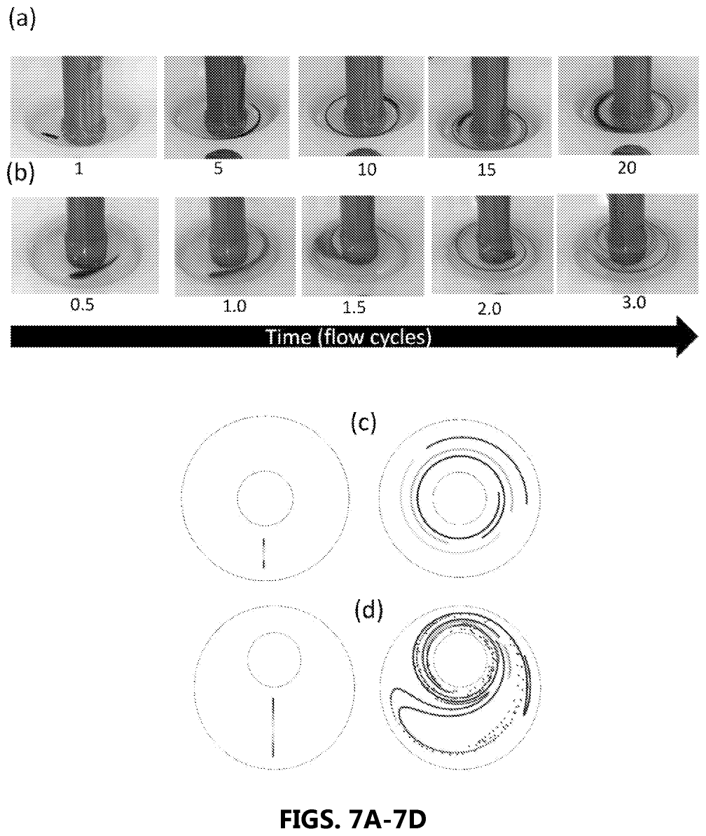

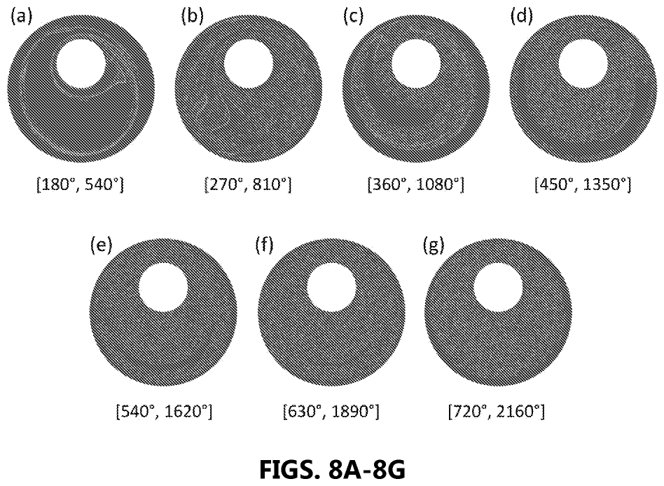

Chaotic printing adheres to the general definition of printing; namely, a process to reproduce a pattern based on a pre-determined template. However, it challenges the paradigms of additive manufacturing. Instead of using moving printing heads and/or a layer-by-layer deposition strategy, chaotic printing relies on the flow itself to do the drawing, much as flow creates a highly convoluted structure when cream is added to a cup of coffee. The choice of materials with high viscosities coupled with low speed mixing enables laminar flow conditions. The selection of specific iterative operational protocols (mixing protocols) originates chaotic flows (FIGS. 7A-7D and 8A-8G) and develops high resolution, predictable, and complex structures that can then be solidified.

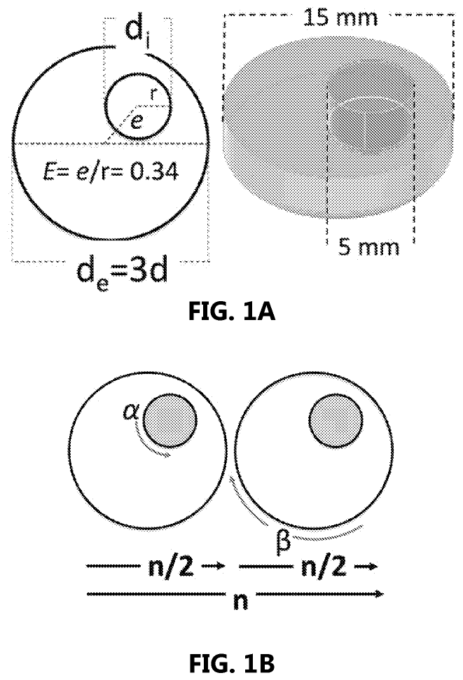

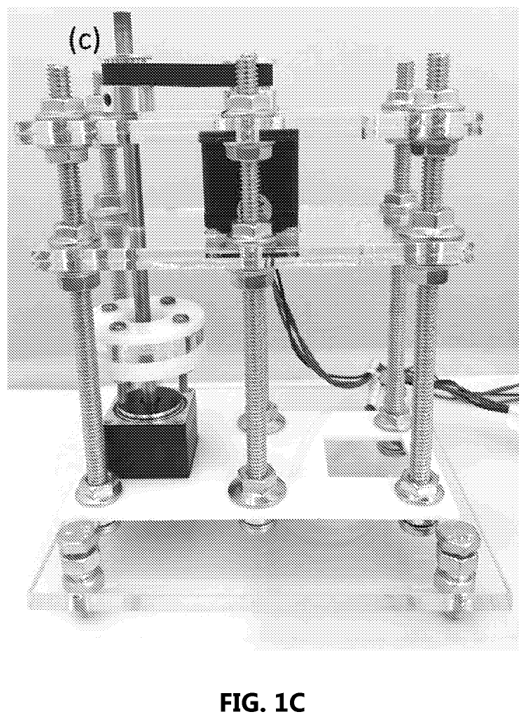

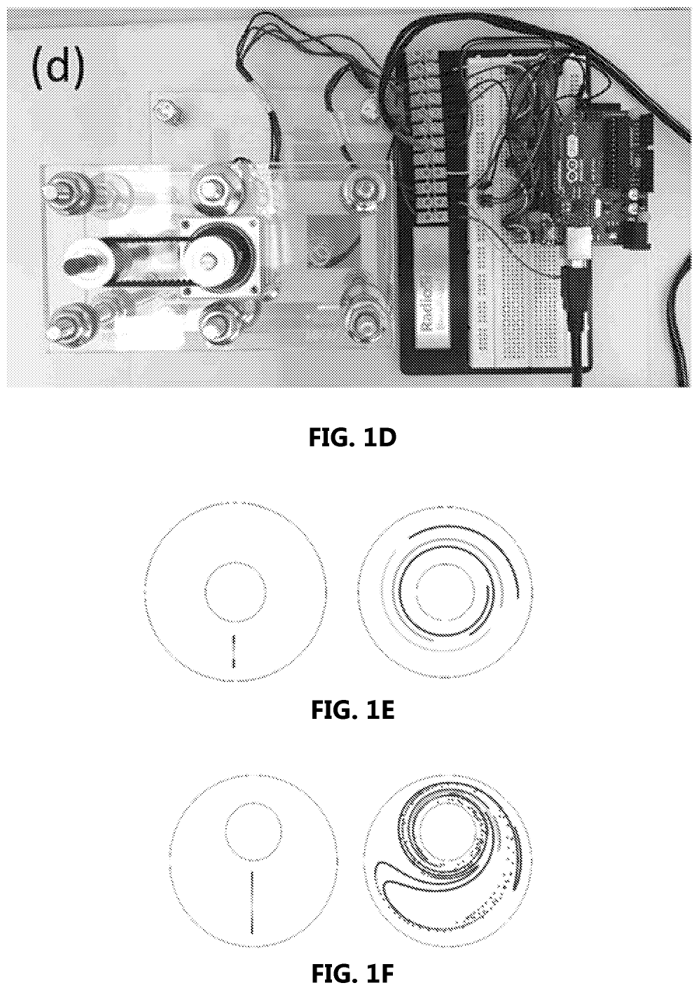

Many chaotic flows have been described; some of these are only theoretical, while others are physically feasible. Our demonstrative model is a finely controlled and miniaturized version of a journal bearing (miniJB) flow (FIGS. 1A-1F). Our miniJB consists of a cylindrical reservoir (outer cylinder) and an eccentrically located shaft (inner cylinder) (FIG. 1A); these slowly rotate alternately (one at the time), and in opposite directions according to a given mixing protocol or recipe (FIG. 1B). A mixing protocol [a1, b1] is fully defined by the counter-clockwise angle of rotation of the external cylinder (a), a momentary stop, and the clockwise angle of rotation of the internal cylinder (b). In this system, we mix at least two components—a viscous fluid polymer and ink(s)—to generate a simple or regular (FIG. 1E) or complex (FIG. 1F) lamellar microstructure.

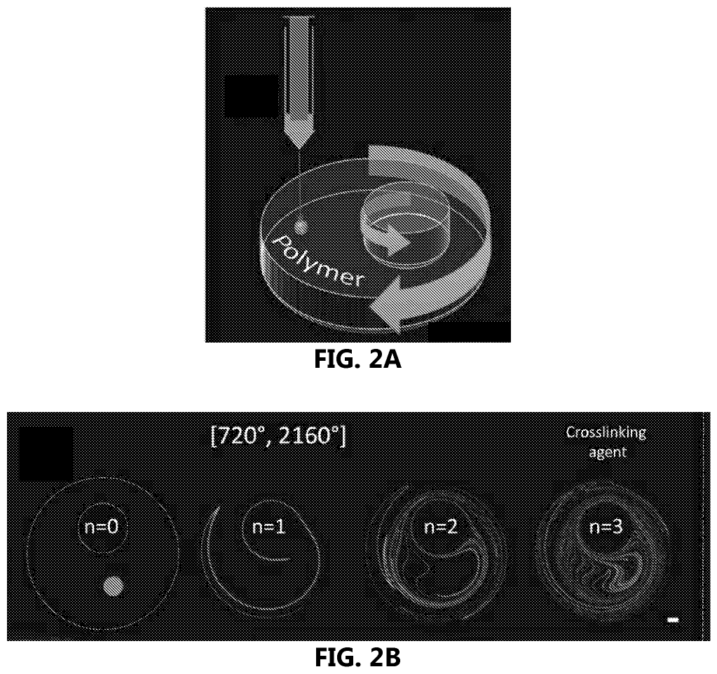

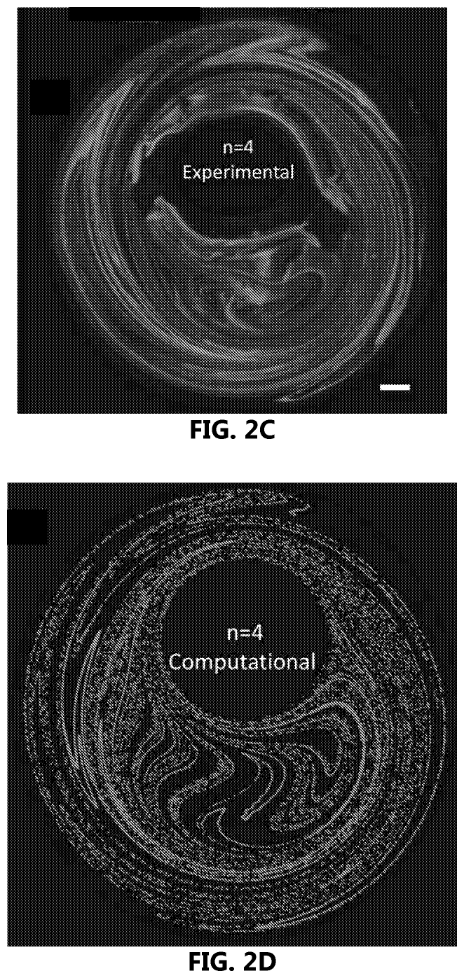

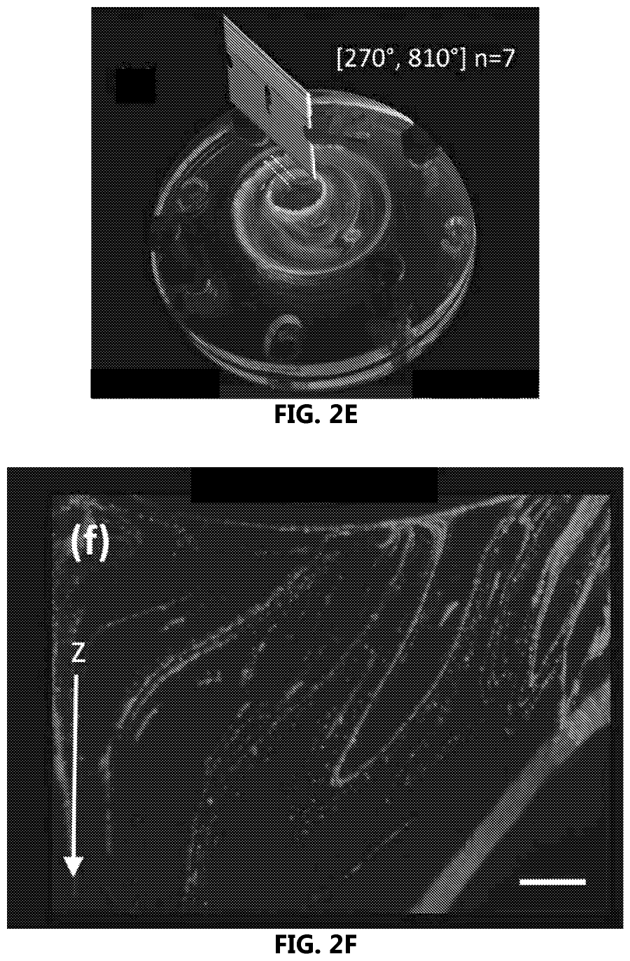

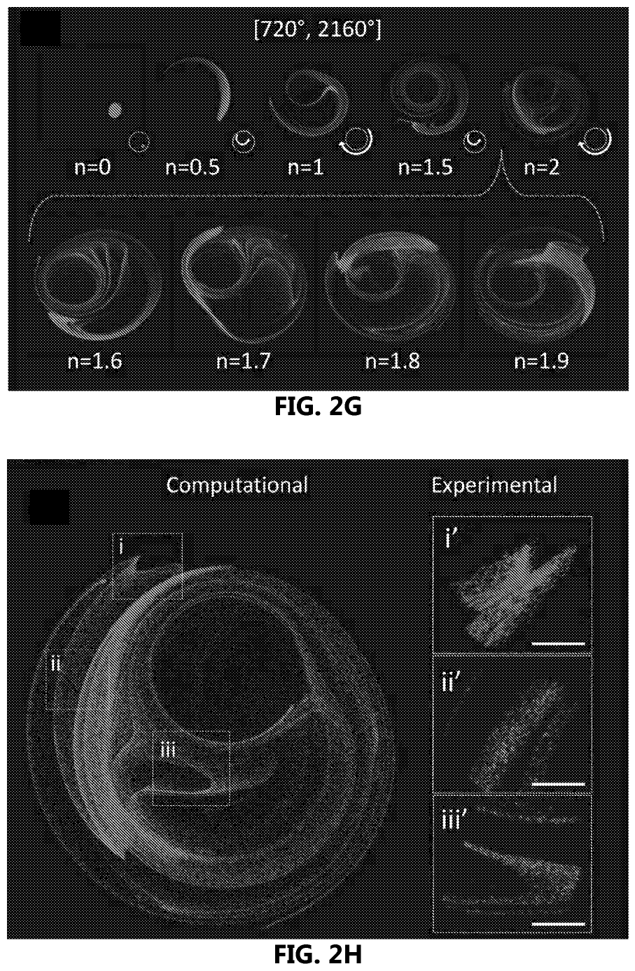

A drop of ink (i.e., a pre-gel drop, a suspension of fluorescent particles, nanoparticles, or cells) is injected into the viscous fluid contained inside the outer cylinder, and the flow [a1, b1] is applied for a number of flow cycles (n) (FIGS. 2A and 2B).

A chaotic flow exponentially deforms and elongates the drop, increasing the interface between the ink and the viscous fluid, and creating a microstructure at an exponential rate (FIG. 1F). The process of creation of the microstructure is guided by an intrinsic flow template (flow manifold) that is characteristic for each mixing protocol (FIG. 8). The resulting microstructure can be stabilized within the polymer by curing or crosslinking after any given number of flow cycles (FIG. 2B). In our experiments, gelatin methacryloyl (GelMA) and polydimethylsiloxane (PDMS) were used as liquid polymers.

FIG. 2C shows a construct fabricated by injecting fluorescent microparticles into a liquid GelMA pre-polymer and allowing the flow to operate for 4 complete flow cycles (n=4). A chaotic flow was induced by a clockwise rotation of 720° of the outer cylinder, followed by a counter-clockwise rotation of 2160° of the inner cylinder [720°, 2160°]. The specific miniJB geometry used induced a globally chaotic flow (FIGS. 8A-8G) created structure by the repeated reorientation and deformation of the fluid, promoting stretching and folding. Such a simple iterative process drives the development of complex structures, and enables the creation of progressively finer patterns, moving from macro to micro and even nano-scales. The final structure was then solidified by simply exposing the GelMA pre-polymer to UV light for 30 seconds.

Since the chaotic flows used to print are deterministic (i.e., they are governed by the Navier-Stokes equations of motion), the process of particle convection and the generation of structures can be modeled (FIGS. 2A-2H). These simulations closely reproduce the behavior of the actual experimental systems; therefore, they enable the prediction of microstructures with specific desired characteristics. For instance, the simple 2D simulations presented in FIG. 2B and FIG. 2D allow an accurate prediction, at a low computing investment, of the structure observed at the top layer of the experimentally obtained construct (FIG. 2C).

The sole observation of this top layer of the microstructure may convey the idea of the existence of a surface 2D flow. However, miniJB flow is capable, by design, of producing highly complex 3D flows (and therefore 3D microstructures). The asymmetry in the boundary conditions at the top and bottom fluid layers—the top layer is a free surface while the bottom one is solid and rotates—creates a fully 3D flow.

FIG. 2E and FIG. 2F show a cross-sectional image of a PDMS-based construct fabricated through chaotic 3D printing (protocol [270°, 810°], n=7); the structure is evidently 3D, and a membrane-like structure is clearly depicted in the Z plane. Each layer of material, from top to bottom, is topologically different and the geometric features of the entire construct are continuous. The 3D nature of the microstructure generated by our miniJB flow system is described by the computational solution of the equations of motion in 3D (FIG. 2G and FIG. 2H). FIG. 2G shows the evolution of the shape of a drop of ink (a cylindrical array of 120 000 massless particles) into a highly convoluted 3D continuous micro-sheet that rapidly invades the flow domain. Our simulations are capable of reproducing, with high accuracy, the characteristic features observed in the experimentally obtained constructs (FIG. 2H).

Printing at a High Resolution and Speed

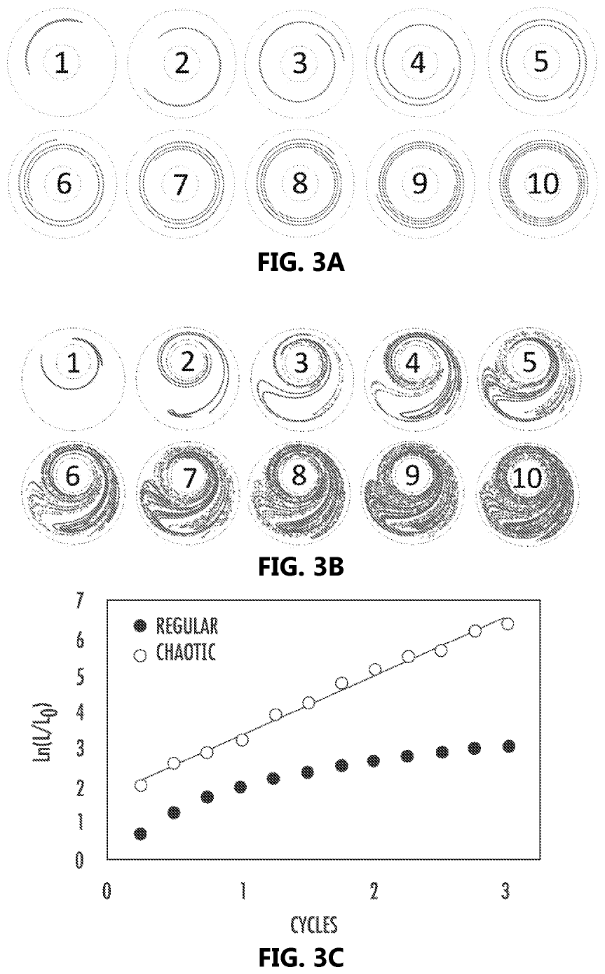

In chaotic flows, stretching proceeds at an exponential rate. Consequently, whereas regular flows produce interfaces that grow linearly (FIG. 3A), chaotic flows develop the structure at an exponential rate (FIG. 3B); in a chaotic flow, any material filament grows exponentially in time, closely following the following model:

(L/Lₒ)=exp(Λn) (1)

where L is the length of the filament after n flow cycles, Lₒ is the length of the filament at an initial time, and Λ is the Lyapunov exponent of the flow.

The Λ value is a quantity that can be used to estimate the speed of advance of the chaotic process. Therefore, Λ conveys a profound physical meaning for a chaotic flow: it describes, within a single value, the overall potential for the flow to stretch the material, cause elongation, and generate the structure. FIGS. 3A-3C describe the determination of Λ, based on 2D numerical simulations.

Every quarter-a-cycle, the evolution of the length of a filament initially located at the center line between the two cylinders was calculated and recorded as they rotated at 5 rpm—this was done both for a globally chaotic miniJB case [270°, 810°] and for a regular flow case [270°, 810°] (centered JB configuration). A Lyapunov exponent value of 1.61 cycle⁻¹ was determined from the slope of the linear version of eqn (1) (FIG. 3C).

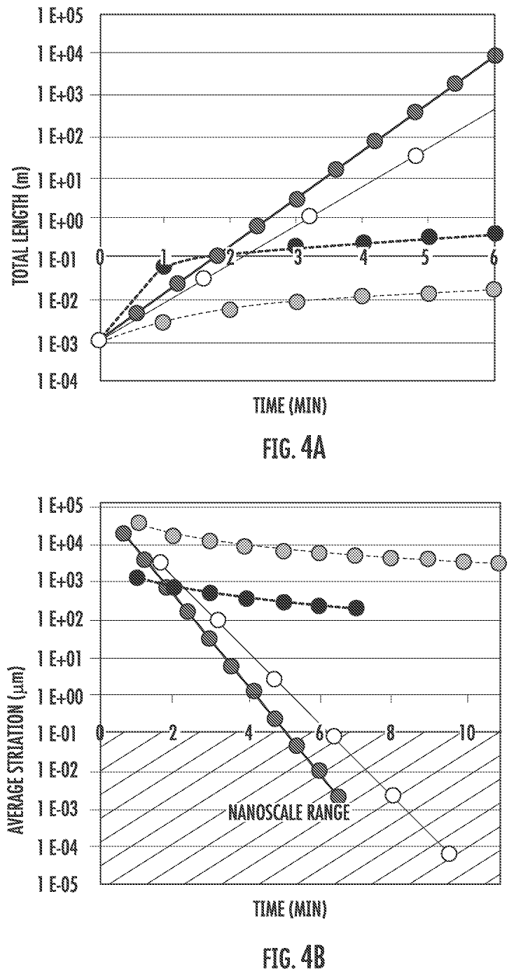

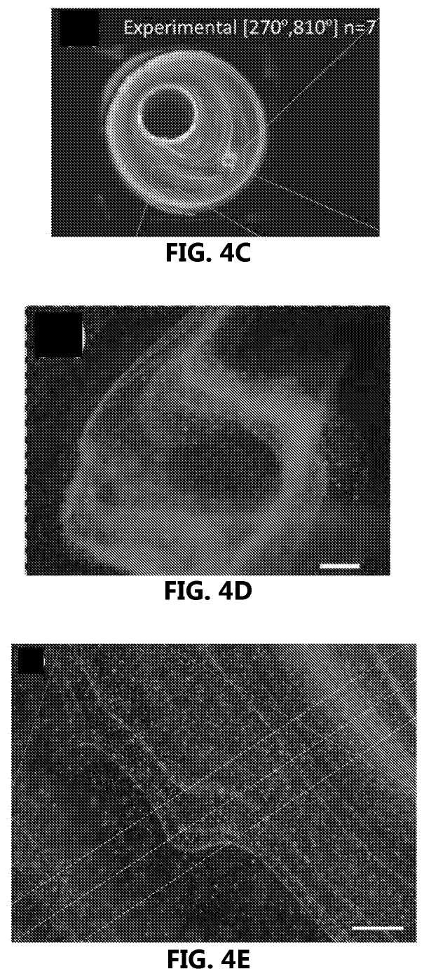

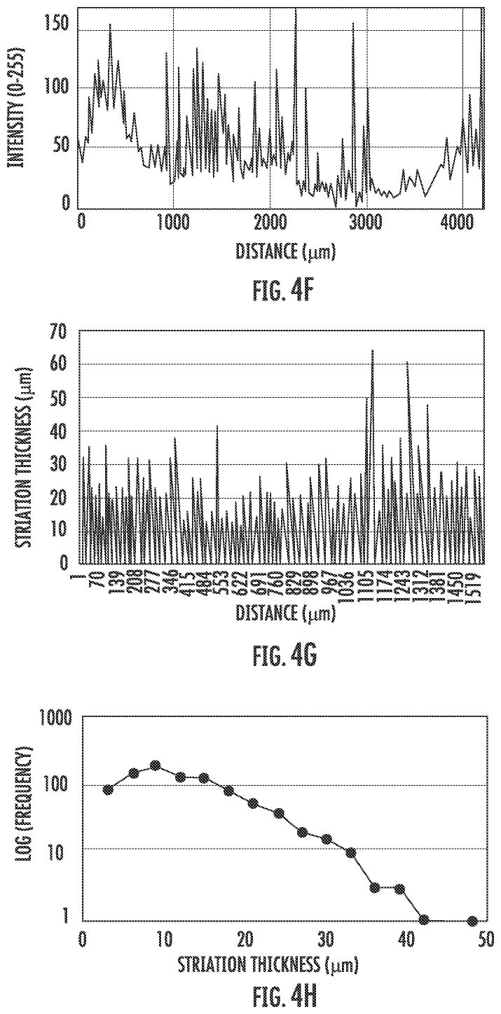

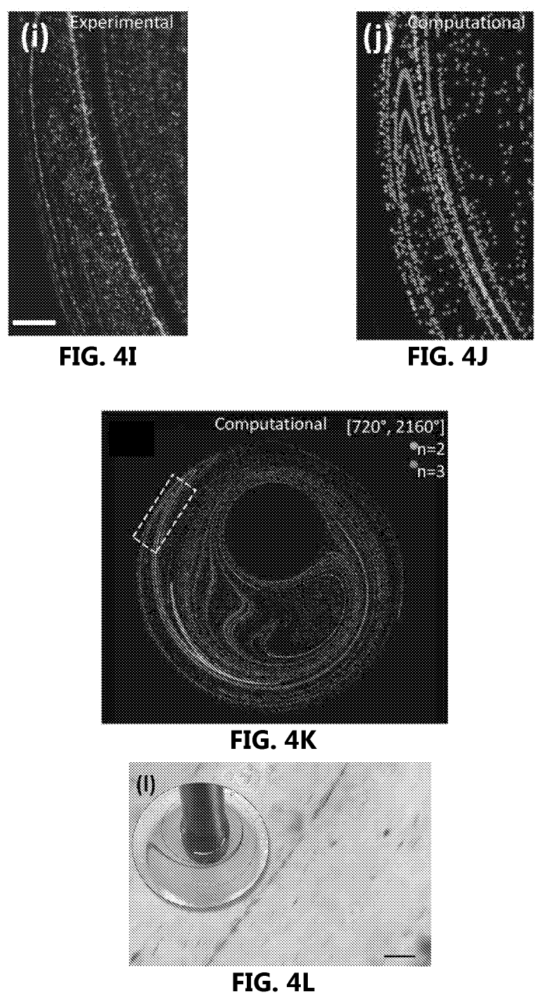

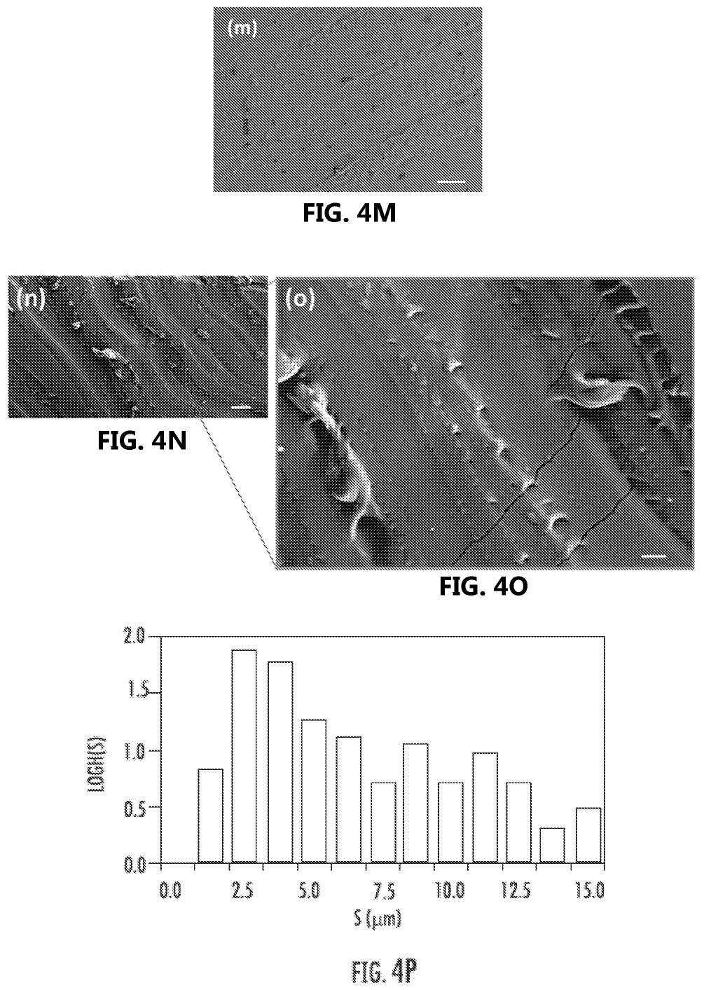

Once the Λ was known, some simple additional calculations were performed. For example, FIG. 4A shows an estimation of the length of a progressively deformed filament that was produced by the repeated stretching and folding of a fluid segment of 1000 mm in a 2D model chaotic flow characterized for L=1.61 cycle⁻¹. This segment could be elongated into a fiber of 3.13 m length after only 5 flow cycles (equivalent to 3 min at our operational speed of 5 rpm). By comparison, an extrusion-3D professional printer prints at a maximum speed of 1.2 cm s⁻¹ for resolutions of 200 μm. The currently available nozzle-based bioprinting techniques exhibit a speed limit between 10 and 50 μm s⁻¹ for resolutions between 50 and 200 μm.

At maximum speed, an average professional 3D printer, and a nozzle-based 3D bioprinter, will print the same segment (3.13 m long) in 43.47 min and 17.38 h, respectively (Table 1).

TABLE 1
Comparison of the length of a printed segment (Ln), the number of striations, and the
average striation thickness (S) at different times generated by a chaotic printer, and an average
3D printer (printing @velocity = 1.2 mm/s).

| | Chaotic printer (A = 2.68 min-i) | | | Professional Extrusion 3D printer | | |
|---|---|---|---|---|---|---|
| time | | number of | | number of | | |
| (min) | Lₙ (m) | striations | \dot{s}(m) | Lₙ (m) | striations | \dot{s}(m) |
| 0.0 | 1.00E − 03 | | | | | |
| 0.6 | 5.00E − 03 | 5.00E − 01 | 2.00E − 02 | 4.32E − 02 | 4.32E + 00 | 2.31E − 03 |
| 1.2 | 2.50E − 02 | 2.50E + 00 | 4.00E − 03 | 8.64E − 02 | 8.64E + 00 | 1.16E − 03 |
| 1.8 | 1.25E − 01 | 1.25E + 01 | 7.99E − 04 | 1.30E − 01 | 1.30E + 01 | 7.72E − 04 |
| 2.4 | 6.26E − 01 | 6.26E + 01 | 1.60E − 04 | 1.73E − 01 | 1.73E + 01 | 5.79E − 04 |
| 3.0 | 3.13E + 00 | 3.13E + 02 | 3.19E − 05 | 2.16E − 01 | 2.16E + 01 | 4.63E − 04 |
| 3.6 | 1.57E + 01 | 1.57E + 03 | 6.38E − 06 | 2.59E − 01 | 2.59E + 01 | 3.86E − 04 |
| 4.2 | 7.84E + 01 | 7.84E + 03 | 1.27E − 06 | 3.02E − 01 | 3.02E + 01 | 3.31E − 04 |
| 4.8 | 3.92E + 02 | 3.92E + 04 | 2.55E − 07 | 3.46E − 01 | 3.46E + 01 | 2.89E − 04 |
| 5.4 | 1.96E + 03 | 1.96E + 05 | 5.09E − 08 | 3.89E − 01 | 3.89E + 01 | 2.57E − 04 |
| 6.0 | 9.82E + 03 | 9.82E + 05 | 1.02E − 08 | 4.32E − 01 | 4.32E + 01 | 2.31E − 04 |
| 6.6 | 4.91E + 04 | 4.91E + 06 | 2.04E − 09 | 4.75E − 01 | 4.75E + 01 | 2.10E − 04 |

These calculations also suggest that chaotic printing can generate finer features faster than conventional linear printers. In our chaotic printing system, any growing segment of interface is accommodated within the finite volume between cylinders, which results in a densely packed microstructure.

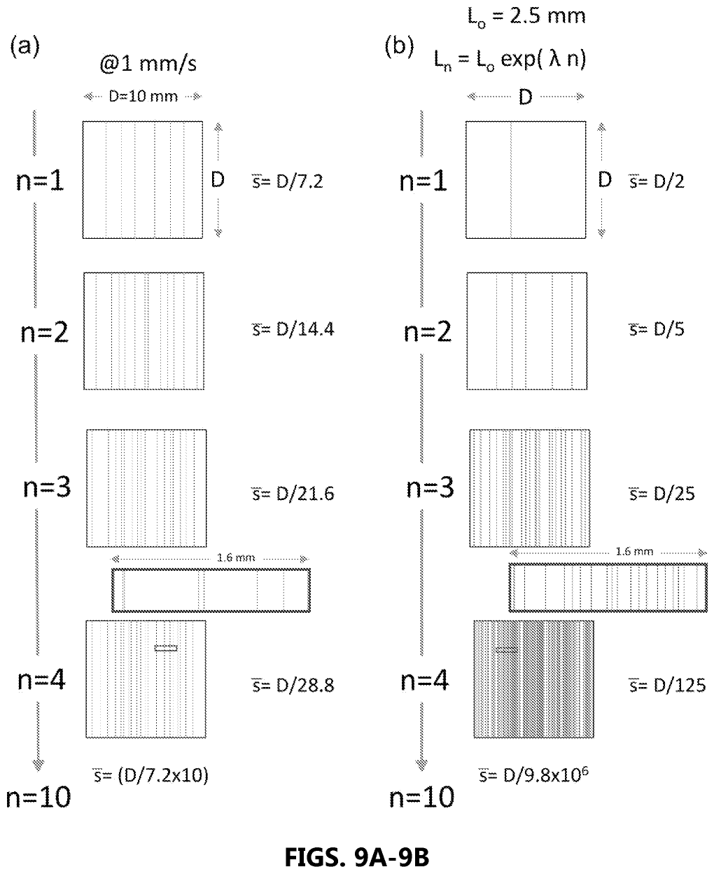

Therefore, this exponential growth rate implies the creation of a great expanse of interface in a small area and in a few flow cycles (FIG. 2C and FIG. 9).

In FIG. 4B, we have calculated the evolution in time of the average striation thickness resulting from the repeated operation of an ideal 2D chaotic printer characterized by Λ §=2.68 min⁻¹, an average speed professional 3D printer printing at 1.2 mm s⁻¹, and a nozzle-based state of the art 3D bioprinter printing at 50 μm s⁻¹ on a square section of 1 cm². Remarkably, our simple chaotic printer can produce an average striation thickness of 10 nm after 10 flow cycles (6 minutes) (FIG. 4B; FIG. 9 and Table 1).

FIG. 4C-FIG. 4E show a PDMS construct obtained by 3D chaotic printing using an ink composed of fluorescent microparticles suspended in PDMS. The multiple sheets of particles were observed in remarkable alignment and packed in a nearly parallel fashion (FIG. 4E).

Based on a simple image analysis conducted by measuring the sum of the length of particle lines per unit of surface area in that particular plane, —7.10 m per cm² of interface was developed in a few flow periods (n=7). The resolution of this microstructure is exceptionally fine. In agreement with our theoretical estimates, in some regions of the flow, the distance between adjacent lamellae (striation thickness) is less than 10 μm (FIG. 4E-FIG. 4H), a value that equals or exceeds the resolution of any 3D printer currently available today. The best resolution achievable with a state-of-the-art commercial 3D printer (i.e., Raise 3D N2) and bioprinter (i.e., Inkredible+ from Cellink) is still on the order of 10 and 50 μm, respectively.

We calculated the distribution of distances between neighboring lines (striation thickness distribution or STD) using image analysis techniques (FIG. 4F-FIG. 4H). Most striation thicknesses reside in the range of 5-20 μm, with a mode peak at 10 μm, following the application of the mixing protocol [270°, 810°] n=7. The shape of the STD, which deviates from that of a normal distribution and is significantly skewed towards low striation values, is a signature feature in chaotic flows.

The STD is not homogeneous throughout the entire chaotic flow domain, with some regions of the flow showing more densely packed structures and the distance between striations much lower or higher than the expected average (FIGS. 4E, 4G, and 4H).

The process of creation of the microstructure in a chaotic flow is governed by a property called asymptotic directionality (AD), which induces a rapid alignment of all the flow vectors in a chaotic flow to the invariant manifold template of the flow. This flow-induced alignment results in the creation of very complex and fine (micro) structures, replicated in time and space, with robust and reproducible statistical properties. The added length of the filament aligns to the previously developed structure, thereby creating, at each flow period, a more complicated microstructure composed of a nested family of parallel curves (FIG. 4I-FIG. 4K). FIG. 4I shows the microstructure generated by the injection of green and red particles at two different times and locations using the miniJB system. Since all injections align to the same flow manifold, we obtained a set of intimately nested curves of different colors. Importantly, our simulations closely predicted this complex microstructure (FIG. 4J and FIG. 4K) resulting from the injection of more than one ink, confirming the predictability of the architecture of these highly convoluted nested surfaces.

Chaotic printing can serve as a versatile nanofabrication platform as well. Calculations in 2D systems suggest that chaotic flows can be used to fabricate at the nanoscale (FIGS. 9A-9B). We investigated this hypothesis further by injecting inks, composed of silver nanowires (200 nm in diameter and 25 μm in length), and carbon and gold nanoparticles (50 nm in diameter), into PDMS in a chaotic miniJB flow, and then curing the resulting structure. The distances between the strings of nanoparticles (striation thicknesses) approached values in the nanoscale neighborhood in a vast portion of the flow domain after n=4. Inspection by optical imaging and/or scanning electron microscopy (SEM) revealed a fine alignment of the sets of nanoparticles under chaotic flows (FIGS. 4L-4O). For example, individual nanowires rotate, similar to fluid vectors, orient and align themselves to the flow template after 4 cycles (FIG. 4L). We also observed alignment of gold nanoparticles to the flow manifold in chaotically printed PDMS constructs (FIGS. 4M-4O), and we measured the STD of the microstructure generated within these constructs. To do this, we cut the PDMS constructs to expose and observe a 2D plane using SEM. We used image analysis techniques to measure the distances between parallel strings of nanoparticles within that plane (FIG. 4P). The main population of striation distances resides in the order of microns (with a peak at ˜2.5 mm) in constructs produced by 4 flow cycles.

Taken together, these results and calculations demonstrate that chaotic flows allow us to “draw” parallel convoluted nanoparticle lines (or surfaces) at the micro- and nanoscale (FIGS. 9A-9B). As such, chaotic printing can be used to be the fabricate highly packed arrays of nanoparticles. Conceptually, the sheets of conductive nanoparticles can be packed at high density within a dielectric or non-conductive material (e.g., PDMS) to fabrication of supercapacitors or simply constructs with tunable or localized conductivity. In addition, the use of high nanowire concentrations can yield long cables (or conductive sheets) fully embedded within polymer constructs.

Chaotic Bioprinting

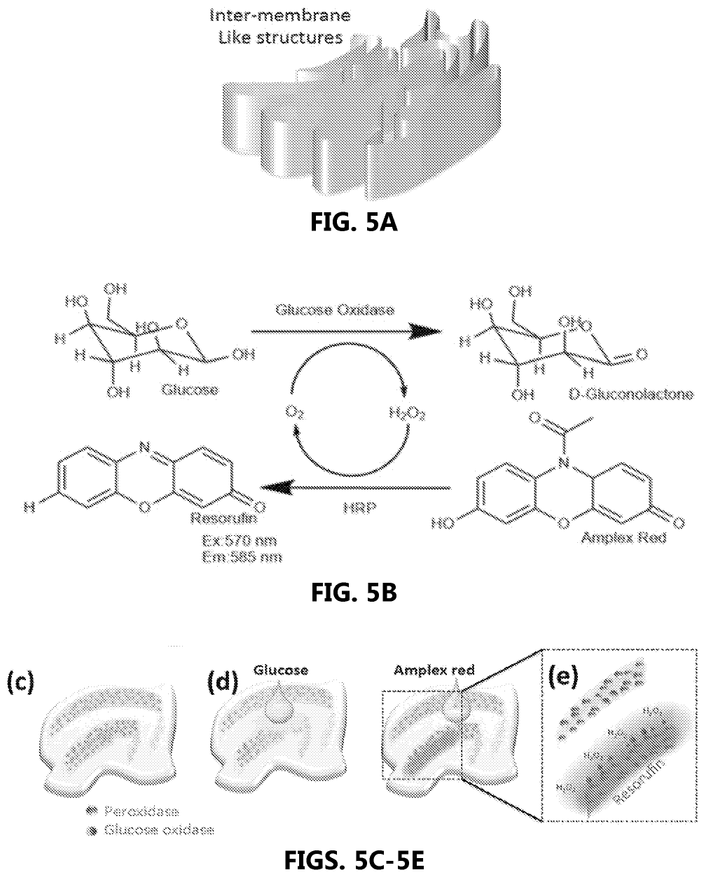

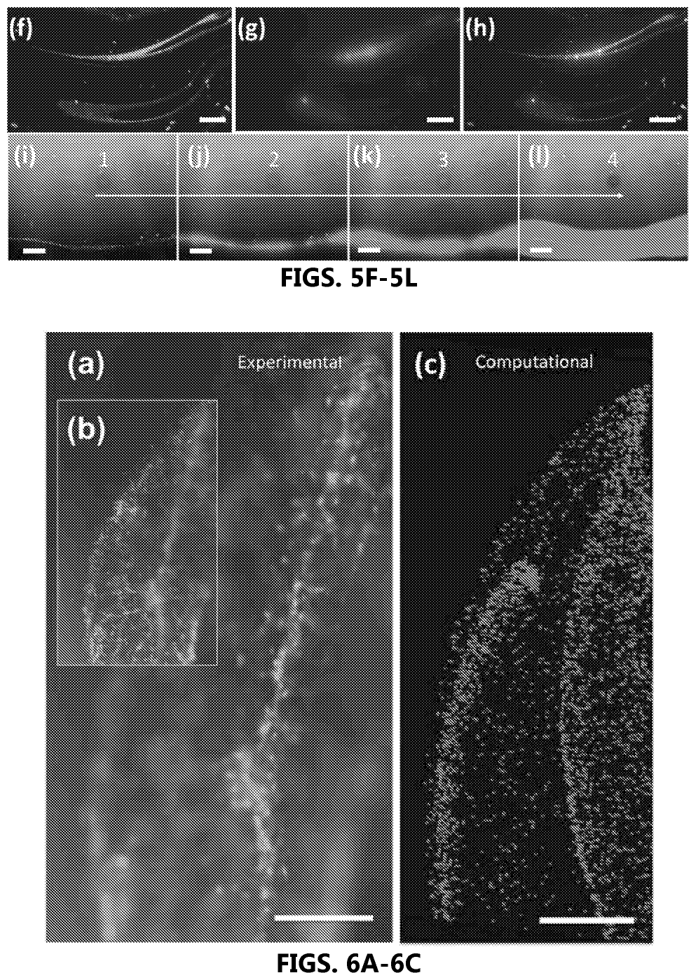

Next, chaotic bioprinting applications were explored. Nature presents us with many examples of highly efficient catalytic surfaces where multiple enzymes are arranged in high density and proximity. This is one of the strategies that cells use to conduct high efficiency conversion processes, such as photosynthesis in thylakoids, energy production in mitochondria, or glycosylation in the endoplasmic reticulum. 3D chaotic printing was used to fabricate convoluted bioinspired bio-catalytic surfaces (FIG. 5A) similar to those presented in FIG. 2F. A system of two sequential reactions, glucose oxidation and hydrogen peroxide conversion, was used as a model (FIGS. 5B-5E). We independently immobilized biotinylated horseradish peroxidase and glucose oxidase on polymer nanoparticles and co-imprinted them in a GelMA pre-gel construct to fabricate a convoluted multi-lamellar structure containing both enzymes immobilized in close proximity (FIGS. 5C-5H).

The addition of glucose to the construct triggered an oxidation reaction mediated by the glucose oxidase to produce hydrogen peroxide. This was then used as a substrate by the peroxidase. The reaction was indicated by the development of red fluorescence due to the oxidation of Amplext red (FIG. 5E). The reaction front was localized in regions where the second enzyme (the peroxidase) was immobilized (FIGS. 5F-5H). The extent of the reaction, as revealed by the intensity of red fluorescence, was proportional to the density of the nanoparticles functionalized with peroxidase (FIG. 5B). The progression of the reaction in time could be followed by successive imaging (FIG. 5I-5L).

The facile creation of convoluted and fine tissue-like structures is an on-going challenge in tissue engineering. Current bioprinting techniques enable the printing of simple cell-laden constructs, where one cell type at a time is printed in one matrix.

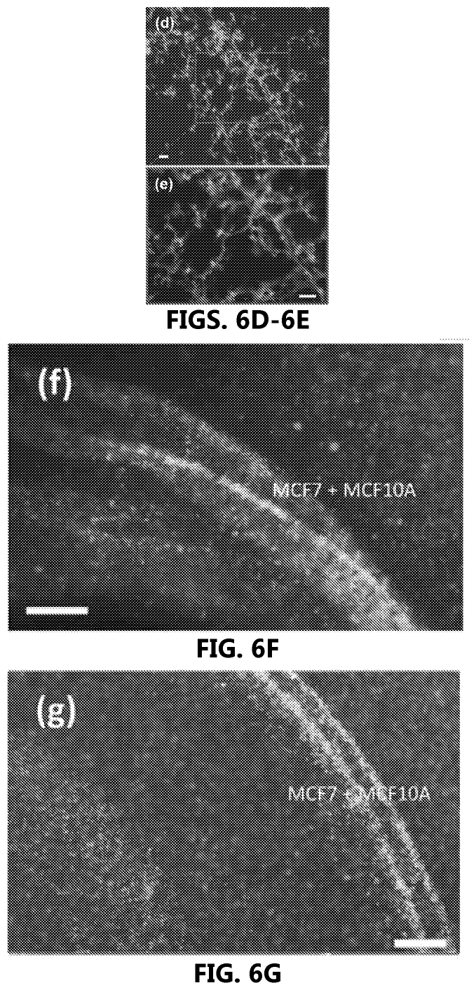

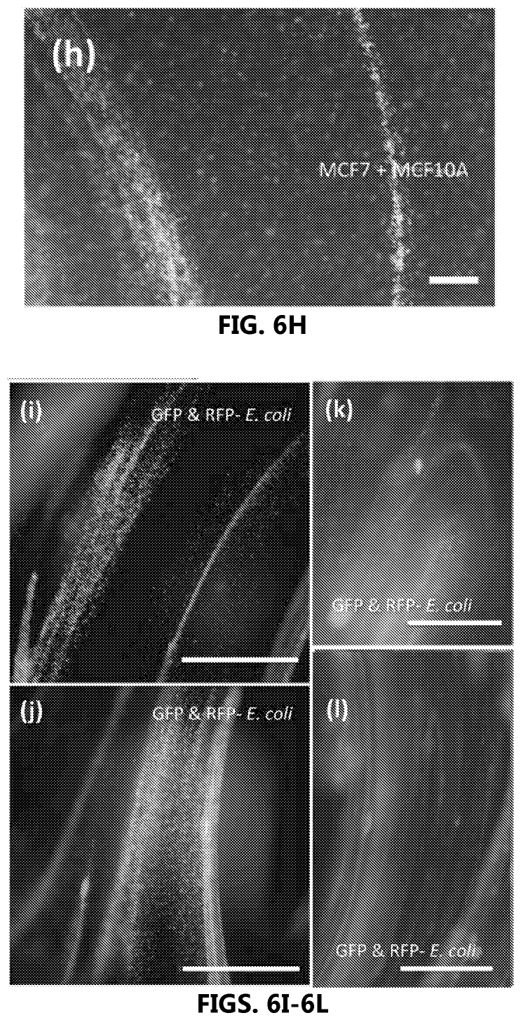

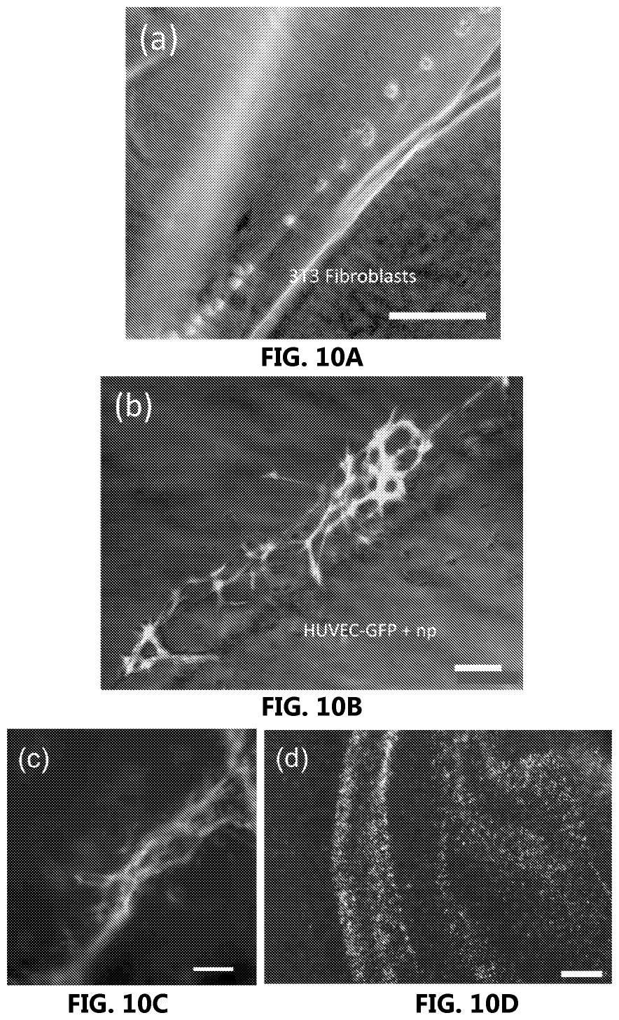

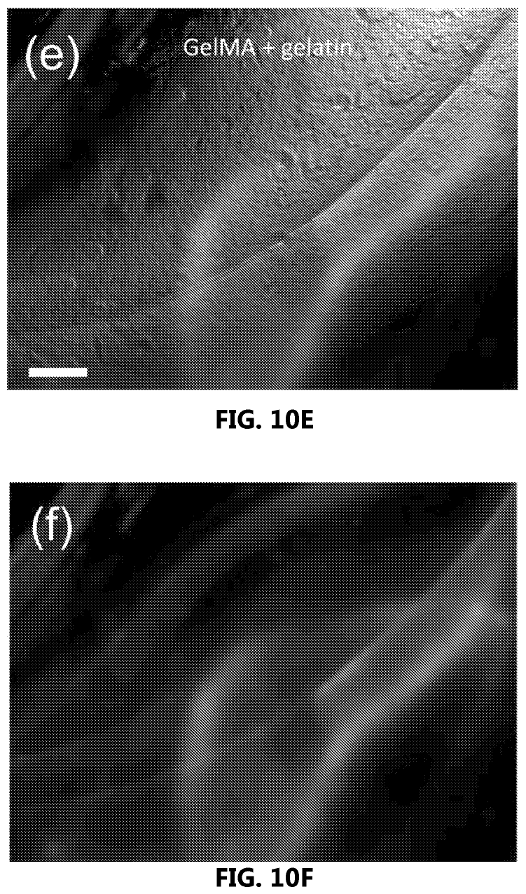

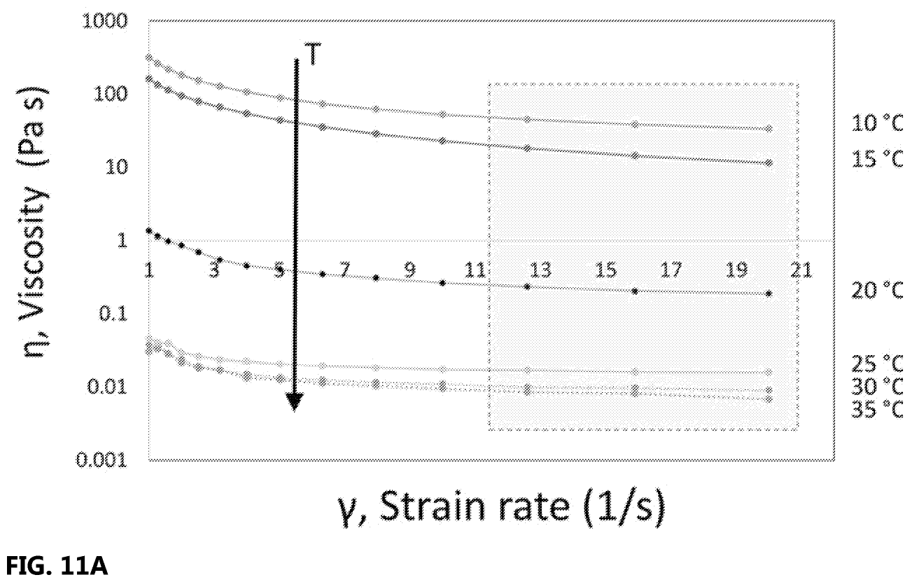

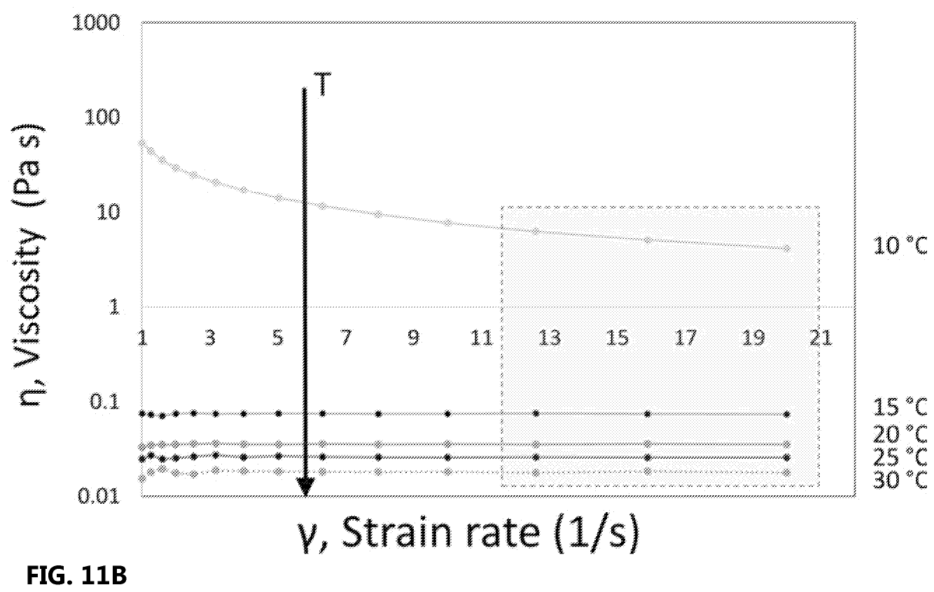

A one-step fabrication of fine cell (or tissue) multilayers with an accurate control of alignment and position is not achievable with the currently available bioprinting techniques, but this level of precise control is an indispensable requirement for the creation of multilayered tissue-like constructs. Our miniJB flow allows the printing of multi-layers of one or more particle type (single or multiple bio-inks), within the same matrix and in a single operation (FIG. 6A-6C and FIG. 10). Each injection rapidly aligns to the manifold of the flow, due to the property of AD. We used inks composed of HUVEC suspensions and GelMA-VEGF (GelMA covalently functionalized with vascular endothelial growth factor or VEGF) to print layers of cells in GelMA matrices. This alignment yields a highly ordered structure that closely resembles the multi-layered tissue structures of complex mammalian tissues (e.g., skin, cancerous tissue encapsulated in healthy organs, pancreatic tissue, and brain pathways). Chaotic bioprinting is cell friendly. The cells are minimally exposed to shear forces during injection with conventional pipettes and later during exposure to laminar flows. We observed mammalian cell viability in the range of 90 to 97% after printing. Cells preserved their original seeding position during the first 96 hours of culture (FIGS. 6A-6B), spread at later times, using the flow structural template, and reached across lines to establish connections between neighboring lamellae to develop complex tissue-like structures (FIG. 6D, 6E and FIGS. 10A-10F, and FIGS. 11A-11B). As shown earlier, the resulting structure can be accurately predicted by CFD simulations (FIG. 6C).

The microstructure fabrication strategy proposed here can be applied to many relevant biological and biomedical engineering scenarios, ranging from the study of fundamental questions related to cell-cell interactions at microbial communities or tissue interfaces to the development of organ-on-a-chip platforms.

FIG. 6F shows a 3D printed construct containing both MCF7 (breast cancer cells, red stained) and MCF10A (noncancerous breast tissue fibroblasts, green stained). A significant amount of interface between the cancerous and normal cells develops after 3 flow cycles. At low cycles, the different degrees of intimacy between cancerous and normal tissue can be observed at different locations within the same construct (FIGS. 6F-6H). This allows the study of the effect of a variety of degrees of spatial interaction between cancerous and healthy tissue under the same experimental conditions. Greater intimacy between different types of cells can also be achieved simply by increasing e number of flow applications. For example, we printed bacterial communities of red (the producers of red fluorescent proteins [RFP]) and green (the producers of green fluorescent proteins [GFP]) Escherichia coli with different degrees of proximity by varying the number of printing cycles. At n=3, E. coli-GFP and E. coli-RFP are planted along different branches of the flow manifold. At n=4, sections populated by the lines of GFP-bacteria are flanked by RFP-bacterial lamella. At higher cycles, a great amount of interface is shared between both cell types (FIG. 6K). Also, the STD (and the degree of intimacy between cell types) can be tuned by selecting different mixing protocols; protocols with higher L generate greater intimacy in fewer cycles (FIG. 6L). The results can be anticipated through simulations or experiments using simpler model systems, such as particles and PDMS. The use of two or more polymers to fabricate composites with long embedded sheets (and therefore high interfacial surfaces) might also have diverse and relevant applications. We conducted chaotic printing experiments in which a drop of gelatin was dispensed in the GelMA pre-gel in our miniJB flow. In these constructs, only the GelMA fluid pre-gel is crosslinked after exposure to UV light; the gelatin sheet, rhodamine stained, remains as a soft gel and serves as a sacrificial component that melts rapidly at 37° C., leaving a convoluted empty space in the form of an embedded continuous microstructure (FIGS. 10A-10F). We envision the use of this simple technique for the fabrication of convoluted sacrificial microstructures that might improve the mass transfer within thick cell-laden constructs or for the design and fabrication of micro-vasculature in tissue engineering applications.

Since the chaotic flows we used are deterministic, the dynamics of the printing process is governed by the equations of motion that are solvable numerically for laminar regime conditions. Therefore, the outcome of the printing process, the resulting microstructure, is predictable for any given chaotic flow system.

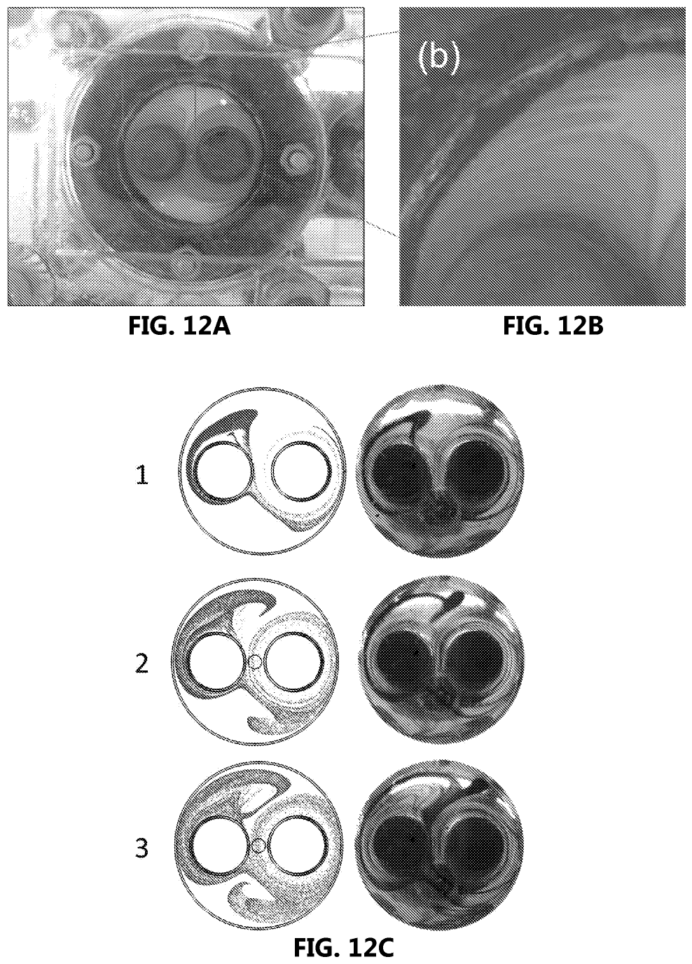

Chaotic printing is a powerful enabler. Remarkably complex microstructures can be fabricated, in a predictable manner, within solidifiable polymers and hydrogels, using chaotic flows generated in a simple “lab-made” printer composed of stepper motors and an Arduino-based controller (FIGS. 1A-1F and FIGS. 12A-12C). We conducted experiments and simulations using another chaotic flow. In principle, any research group would easily be able to produce a version of a chaotic printer with a minimum investment. We have mainly presented 3D printing experiments and simulations using different JB protocols. However, in principle, any realizable chaotic flow could be used as a chaotic printer.

Conclusion

Chaotic printing is a technological platform rooted in strong fundamental concepts. Chaotic flows exhibit an ability to create a self-similar microstructure at an exponential rate due to their inherent properties, which include the existence of a unique flow intrinsic template and AD. The iterative character of a chaotic flow enables the creation of progressively finer patterns, moving from macro to micro and nanoscales. Ink drops will rapidly elongate by the action of the chaotic flow to produce 3D sheets with a total length that will grow exponentially and rapidly to reach hundreds of centimeters after a few flow cycles. In volume preserving systems, this exponential increase in length aligned to the flow manifold will necessarily imply a contraction in other dimensions.

Experimental Methods

Experimental set-up. We built a miniJB system composed of a cylindrical reservoir 1.5 cm in diameter made of poly(methyl methacrylate) (PMMA) (referred to as the external cylinder) and an eccentrically located cylindrical shaft 0.5 cm in diameter (referred to as the internal cylinder). The degree of eccentricity (E=e/r) was set at E=0.34, where e is the distance between the shaft center line and the system center line and r is the diameter of the external cylinder. The rotations of the internal and external cylinder were independently controlled using two mini stepper motors (Nema 11, 3.8V, 0.67 A, Model 11HS12-0674S) controlled by an electronic system consisting of an Arduino UNO module (Arduino, Italy), an electronic card for each motor (EasyDriver; stepper motor driver, SX09402, Sparkfun Electronics, USA), and a 12 V power adapter-converter (US Plug AC 100-240V to DC 12 V 2A) (FIGS. 1A-1F). Experiments conducted using PDMS were performed at rotational speeds of 3 RPM (18° s⁻¹) for both the inner cylinder and the outer reservoir. Experiments using GelMA were performed at rotational speeds of 5 RPM (30° s⁻¹) for both the inner cylinder and the outer reservoir. We used different geometrical configurations and mixing protocols to produce regular, partially chaotic, or globally chaotic structures (FIGS. 7A-7D and FIGS. 8A-8G). A concentric configuration was used to produce fully regular flows, in which stretching is linear. The cylinders were eccentrically located for the fabrication of partially chaotic or globally chaotic structures. In all cases, the mixing protocol consisted of alternating rotations, in opposite directions, of the external and internal cylinders. The angle of rotation of the external cylinder (α°), and the angle of rotation of the internal cylinder (β°), fully define a mixing protocol: [α°, β°]. The mixing protocol is repeated a number of flow cycles (n); a flow cycle is a complete implementation of one rotation of the external and one rotation of the external cylinder.

Varying the mixing protocol in eccentric configurations then permits the selection of partially or practically globally chaotic flow, in which flow filaments stretch exponentially along the intrinsic flow template or manifold (FIGS. 7A-7D and FIGS. 8A-8G). Two chaotic JB recipes were primarily used in this work: (a) [270°, 810°]; and (b) [720°, 2160°]. Mixing protocols were applied for a number of flow cycles (n), ranging from n=3 to n=7.

Printing experiments. Our printing experiments consisted of dispersing small volumes of a viscous suspension or solution (an ink) into a viscous fluid (matrix). For most experiments, the ink injection was located below the liquid surface of the JB reservoir, approximately at the center line of the system and at the mid-point of the gap between the internal cylinder and the internal wall of the external cylinder. We conducted sets of experiments using different cross-linkable or curable materials and diverse “inks” to illustrate the characteristics of the constructs produced by the application of this technique, to confirm its robustness and flexibility and to demonstrate its potential in various applications. PDMS and GelMA were used as the main matrix fluids for printing experiments. Diverse inks were used to illustrate different applications of 3D chaotic printing: suspensions of fluorescent particles to reveal the microstructural features of chaotic and regular flow recipes; gelatin to fabricate sacrificial convoluted empty spaces within GelMA constructs; HUVEC and 3T3 cell suspensions in GelMA-VEGF to print tissue-like microstructures; MCF7 and MCF10A cells to bioprint constructs where cancerous and healthy breast tissues co-exist; green florescent protein (GFP) and red fluorescent protein (RFP) Escherichia coli bacteria to print bacterial consortia sharing different degrees of interface; and biotinylated enzymes (e.g., glucose oxidase and peroxidase; Sigma Aldrich; USA) immobilized to streptavidin-functionalized nanoparticles (MagVigen™; Cat#21005; USA) to print biocatalytic membranes in GelMA constructs; and gold and carbon nanoparticles (Sigma Aldrich; USA), and silver nanowires (ACS Materials; cat # Agnws-200; USA) to print microstructures using nanoparticles.

The microstructure fabricated by the mixing process was preserved by curing at room temperature for 24 hours for the PDMS construct, or UV light exposure (30 seconds; output power of 850 mW at a distance of ˜5 cm, using an OmniCure® S2000 system) for GelMA based structures.

Preparation of Polymer. PDMS was prepared by mixing a PDMS pre-polymer solution and curing solution (Dow Corning Sylgard 184 Silicon Encapsulant, clear kit, Dow Corning; USA) in a 9:1 volumetric ratio. GelMA (5%) was prepared by dissolving 150 mg of freeze dried GelMA, prepared as described in literature, in a solution consisting of 15 mg of photoinitiator (Ciba Irgacure® 2959, from BASF, Germany) in PBS. This GelMA suspension was vortexed repeatedly during incubation at 70° C. until dissolution. GelMA-VEGF (GelMA covalently functionalized with VEGF) was prepared by covalently conjugated to GelMA using the N′-ethyl-carbodiimide hydrochloride (EDC)/N-hydroxysuccinimide (NETS) coupling chemistry. Before coupling, GelMA was reacted with an excess amount of succinic anhydride at 50° C. to convert most of the lysine amine groups into carboxylic acid groups and minimize side reactions during EDC coupling. Carboxylic acid groups were then activated by reacting with EDC/NHS at room temperature, and VEGF conjugation was achieved by the formation of amide linkages. Gelatin was prepared by dispersing 5 g of porcine skin gelatin (Sigma-Aldrich, USA) in 95 g of phosphate buffer solution (PBS) (Gibco™ Cat No. 14200-075; Thermo Fisher, USA), incubating 60 minutes at room temperature, and heating the solution in a water bath at 70° C. until dissolution. The gelatin solution was used at room temperature in the chaotic printing experiments.

Microstructure analysis. The microstructural features of the fabricated constructs were analyzed by optical microscopy using a fluorescence optical microscope (Zeiss, USA), or by confocal microscopy. Nanostructure alignment was verified using a scanning electron microscope (SEM) (Quanta 200 FEG, FEI™) under high vacuum or a Merlin High-resolution SEM (Zeiss, USA). Samples were gold or carbon coated with a Gatan High Resolution Ion Beam Coater.

CFD Simulations. We conducted simplified 2D simulations to determine the value of Λ for different JB flows, as well as 3D finite element method (FEM) simulations to characterize the full 3D dynamic behavior of our miniJB system. The A value was estimated by idealizing the JB system as a 2-dimensional (2D) flow and a FEM was implemented in COMSOL Multiphysics 4.4 using rotating machinery, laminar flow, and particle tracing for fluid flow physics. Different positions of injection were compared and Λ at different flow locations was estimated by conducting a simulation with 8 line-increments on the centerline passing the centers of two circles. Simulations were performed at a rotational speed of 5 RPM (same rotational speed used in experiments) for both the inner and outer cylinder walls; the fluid kinematic viscosity was set at 1 Pa s and both the solid particles (d=10 μm) and the matrix fluid were considered equal to 1.0 g cm-3. For the purpose of the simulation of the flow field and microstructure of the 3D cases, we solved the Navier-Stokes equations of motion in a 3D grid build in COMSOL Multiphysics 4.4. using rotating machinery, laminar flow, and particle tracing for fluid flow physics. The geometry of the experimental JB system was closely matched. The simulation was discretized with reasonably fine free triangular elements, and mesh sensitivity studies were conducted to ensure consistency of results.

The rotating machinery formulates the Naiver-Stokes equations in two rotating coordinate systems and couples them within a fixed coordinate system. In particle tracing physics, we used Newtonian formulation for the particle movement in the fluid domains as:

$$\frac{d}{dt} ( {m}_{p} v ) = {m}_{p} F ( u - v ) \qquad ( 2 )$$

where u is the fluid velocity, mₚ is the particle mass, v is the particle velocity, and F is the drag force per unit mass. The boundary conditions for the fluid flow were used as no-slip, which means that the velocity of the wall is equal to the velocity of the contacting flow.

We conducted simulations of different JB chaotic flows by solving the equations of fluid motion using finite element techniques, and we tracked sets of tracer particles within these flows to simulate the process of dispersion of drops of ink. This allowed a highly accurate prediction of the microstructure produced from the application of a particular mixing protocol.

Notes on chaotic printing using JB flows. The occurrence of chaos in the JB system, or in any potentially chaotic system, depends on geometry factors and operational conditions. In the JB flow, the off-centered location of the inner cylinder is a mandatory requisite for producing chaotic flows (FIGS. 7A-7D). Concentric JB configurations result in regular flows, where lines, surfaces, or volumes (i.e., filament segments or drop interfaces) will undergo only linear stretching in the direction of the flow lines (FIGS. 7A and 7C). However, in eccentric (off-centered) configurations, the geometric symmetry of the system is broken, resulting in an additional degree of freedom. This simple geometric change enables the generation of chaotic flows under specific operational protocols (FIGS. 7B and 7D).

For example, FIGS. 8A-8G shows simulation results that demonstrate the structures rendered by the application of different flow protocols. In this work, a chaotic protocol is designated as two angle values within square brackets (i.e., [α°, β°]), where α is the number of degrees that the outer cylinder is rotated and β is the number of degrees that the inner cylinder is rotated.

On the calculation of the Lyapunov exponent. Any material drop exposed to a chaotic flow will exponentially stretch—and consequently elongate—along the skeleton of the flow (or invariable manifold). Any chaotic flow has a well-defined, but heterogeneous, stretching field; in any location of a chaotic flow, the stretching intensity can vary by several orders of magnitude. As an example, the simulated evolution of filaments positioned in several different flow locations in regular (FIG. 7C) and chaotic (FIG. 7D) protocols aligns to the same flow manifold but is not identical (i.e., it proceeds at different speeds, particularly during the first flow cycles). However, a common and useful way to characterize the overall (or average) intensity of a chaotic flow, that is how effectively a chaotic flow creates structure, is by determining an average A value.

Several strategies can be used to calculate an average Lyapunov exponent. One meaningful way is based on following the deformation of a material line (i.e., a filament composed of thousands of points) in the flow. FIGS. 3A-3C describe the determination of Λ in detail, based on 2D computational simulations. The units of the Lyapunov exponent (Λ) can be converted from cycles to seconds simply by considering the speed of rotation of the cylinders [RPM] and the angle of rotation of the mixing protocol. For instance, at 5 RPM (the rotational speed used in our experiments), Λ=1.61 cycle-1=2.68 s-1 for the mixing protocol [270°, 810°]. At 5 RPM, Λ=3.50 cycles-1=2.18 s-1 for the mixing protocol [720°, 2160°].

Assessment of microstructure properties based on the value of the Lyapunov exponent. Chaotic printing enables an exponentially fast, facile, and highly precise fabrication of long sheets of material and fibers in short times. This capability is not achievable by any other currently available printing technique, where the printing process is linear. The length and diameter of the stretched filament resulting from different experimental JB flows can be approximated based on the A value and some simplifications.

Modeling stretching of a segment into a fiber. The following exercise assumes that the chaotic flow deforms and stretches a segment of 1 m of length into a fiber. Let us consider a model 2D chaotic flow with a Λ=1.61 cycle⁻¹ (Λ=2.68 min⁻¹ at 5 RPM), similar to the JB flow depicted in FIGS. 3A-3C. The total length of any vector within the initial spherical drop (i.e., its diameter) will then elongate at a rate defined by the Λ value: Ln=Lo exp(Λn). Then, the length of a fiber will grow as dictated by the Lyapunov exponent.

As we have shown, both experimentally and computationally, the 3D JB chaotic flows will produce shapes that are more convoluted than simple filaments. Nevertheless, this scenario is a first approximation to illustrate the remarkable potential of a chaotic flow to elongate materials, and produce surface area. For example, chaotic printing creates structure much more rapidly than currently available nozzle-based techniques, which have speed limits of between 10 and 50 μm s-1 for resolutions between 5-200 μm 8. At 50 μm s⁻¹, a nozzle-based 3D bioprinter will print a segment 3.13 m long in 17.38 hours, with a modest resolution of 200 μm. A professional 3D printer, working with standard thermoplastics such as ABS or PLA, will perform the same task in 43.47 minutes at a resolution of 200 μm. That same length of filament can be obtained by chaotic printing in 6 minutes (Table 1).

Modeling creation of high resolution microstructure. In chaotic printing, the exponential increase in the amount of interface implies that the distance between consecutive lamellae decreases rapidly because the flow occurs in a closed system. Next, we compare the performance of a chaotic printer versus a hypothetical extrusion bioprinter (which is 10 times faster than currently available bioprinting technology) in terms of their ability to create highly packed microstructures in a small area. First, we consider a linear printing process (such as the one conducted by an extrusion bioprinter) operating in a square area D×D, where D=10 mm (FIGS. 9A-9B). The process of creation of structure will be also linear, since its speed is constant in time. For example, let us analyze a printing process with a printing speed of 1.2 mm s⁻¹ (while noting that professional printers perform at this speed, and the fastest extrusion bioprinter available today prints at 100 μm s⁻¹). After 1 minute, this printer will print a filament 72 mm long, which is 7.2 times the characteristic length D. This speed will be sustained for the rest of the printing operation. Therefore, the total length of the printed filament will be 7.2, 14.4, 21.6, and 28.8 D after 1, 2, 3, and 4 minutes, respectively. The characteristic length scale of the squared system, (i.e., the length of one of the sides of the square) will then be dissected by 7, 14, 21, and 28 lines during the first four minutes of the printing operation. The average striation thickness (the average distance between neighboring printed segments) after 1, 2, 3, and 4 minutes will be D/7=1428, D/14=714, D/21=476, and D/28=357 μm, respectively.

Now consider a chaotic 2D model system operating in the same square 1 cm² area. Any linear segment will elongate at an exponentially fast rate under the action of the chaotic flow. An exponential elongation in any area or volume preserving flow implies an exponentially fast and organized decrease in length scales. For example, in a flow with Λ=1.61 cycle⁻¹ (Λ=2.68 min⁻¹ at 5 RPM), similar to that exhibited by the JB flow depicted in FIG. 3, an initial segment of 2.5 mm will map into a filament of 1, 5, 25, and 125 mm after 1, 2, 3, and 4 successive flow cycles. The characteristic length scale of the square system (i.e., the length of one of the sides of the square) will then be dissected by 1, 5, 25, and 125 lines for the first four flow cycles, respectively. The average striation thickness after 1, 2, 3, and 4 minutes will be D/1=10000, D/5=2000, D/25=400, and D/125=80 μm, respectively (Table 1). Similarly, Table 1 presents a comparison of the length produced, the number of striations, and the average striation thickness generated by a chaotic printer and an average professional 3D printer operating at a speed of 1.2 mm/s (˜10's of cm³/h).

The linear printing process is faster during the first two minutes. Ultimately, and soon, the exponential nature of chaotic printing surpasses the ability for microstructure creation of the linear printing process. After 6 minutes, the chaotic printing scheme will generate close to 1 million striations in the squared box (generating an average striation thickness of 10.2 nm). In the same time frame, the linear printing process will have generated only 43 striations, generating an average striation thickness of 230 μm). Note that currently, the maximum resolution physically achievable by a state of the art 3D printer is 5 μm. This limit is surpassed by a chaotic printer after only 4 minutes of printing time.

Chaotic bioprinting of cell-laden constructs. Diverse tissue-like structures can be designed and fabricated simply by varying key parameters in chaotic printing (i.e., choosing between a chaotic, partially chaotic, or regular flow, varying the number of inks, or the seeding cell density). We envision that chaotic printing can be applied to fabricate systems that will enable the study of fundamental questions related to cell-cell interactions at material interfaces with different degrees of vicinity. We chaotically bioprinted different mammalian cell lines in GelMA hydrogel 1 constructs to illustrate some aspects of the technique (FIGS. 5M, 5N, and 5P). GelMA is a photocrosslinkable material that has been widely used in tissue engineering applications: it contains cell binding domains, it is biodegradable, and it is amenable to microfabrication.

Chaotic flows can be used effectively to provide efficient separation and encapsulation of individual 3T3 fibroblasts along a flow line, thereby enabling single cell studies. Varying the initial cell density also allowed control of the cell density along the manifold. In a chaotic flow, vectors (or ink particles) located at different locations in the system experienced different degrees of stretching. The local values of stretching dictated the distance between particles. In low stretching regions, the cells remained closer, while in high stretching locations, they were more widely spaced. Chaotic printing can be effectively used to create ad hoc local microenvironments for cell growth.

For example, endothelial cells were chaotically co-printed with nanoparticles functionalized with VEGF (i.e., the ink was composed of a mix of cells, 5% GelMA, and VEGF attached to nanoparticles) to favor directed and localized cell proliferation and spreading along the manifold lines. After five days, the cells grew and spread within the GelMA construct.

The cell density within the ink to be used and the crosslinking conditions after printing are additional important considerations for chaotic bioprinting. A set of appropriate conditions in terms of these parameters can be determined for each printing experiment. In general, we obtained good results when we used cell densities of 1×10⁷ to 1×10⁹ cells mL⁻¹ and when we closely matched the viscosity and density of the matrix fluid (e.g., by using the same matrix liquid to suspend cells or particles for ink formulation). Other considerations become important for the bioprinting of specific cell lines. For example, we observed long term survival (up to 15 days), but no cell spreading, in a construct printed using human umbilical vein endothelial cells (HUVECs) in GelMA. Spreading was only observed when the inks contained GelMA and nanoparticles functionalized with VEGF, or GelMA covalently functionalized with vascular endothelial growth factor 2 (GelMA: 1/10 GelMA-VEGF) (FIGS. 6A, 6B, and 6D-6E).

In bioprinting experiments, the co-injection of different cell types offers great potential for the fundamental study of cell-cell interactions at different degrees of contact. For example, we have conducted experiments where an ink of breast fibroblast cells in GelMA was bioprinted in a suspension of cancer cells in GelMA. In these experiments, MCF7 cells (1.5×10⁶ cells mL) were mixed with 5% GelMA and 700 μL this cell suspension was dispensed in the printer reservoir, while 5 μL of an ink composed of 5% GelMA and MCF10A cells (0.8×10⁶ cells mL) was initially injected.

Chaotic bioprinting demands careful control of some operational parameters, mostly related to the matching of the rheology between the matrix liquid and the ink and to the conditions of crosslinking. Rheology is an important consideration for chaotic printing; the outcome of chaotic printing is predictable for a Newtonian liquid in a laminar regime. GelMA is a complex polymer mixture composed of fragments of collagen molecules of different sizes. However, GelMA pre-gels (before crosslinking) exhibit a Newtonian behavior in a window of temperatures, concentrations, and strain rates, and possess a conveniently moderate-to-high viscosity that makes them an attractive choice for chaotic bioprinting.

Alternative experimentally feasible chaotic flows. Most of the results presented here were produced using the miniJB flow system described previously. However, chaotic printing can be extended for use with any other experimentally feasible chaotic flow.

For example, FIGS. 12A-12C illustrate the use of another system—an experimentally realizable Blinking Vortex—to generate highly packed structures in viscous materials. We have also conducted successful chaotic printing experiments using static mixers, specifically the Kenics mixer. The Kenics mixer is a static mixer that is used in the process industries to mix liquids in turbulent and laminar regimes. In laminar flows, the Kenics mixer develops chaotic flows by a mechanism of splitting and stretching.

### Example 2. Chaotic Printing: Using Chaos to Fabricate Densely Packed Micro- and Nanostructures at High Resolution and Speed

Introduction

Multi-material and multi-layered architectures achieve functionality and/or performance that are not achievable with monolithic materials. Moreover, the functionality and performance of multilayered composites is frequently determined by the proximity, indeed the density, of the constituent layers. Multilayered materials with a high amount of internal surface area can yield higher capacitances in supercapacitors, elevated mechanical strength and fatigue resistance, better sensing capabilities, or improved energy-harvesting potential. A multi-lamellar architecture that features highly accurate control of surface geometry and surface area is also desirable in applications related to the controlled release of pharmaceuticals.

Multilayered structures are particularly relevant in nature and in biological applications. Indeed, one of the most pressing challenges in biofabrication is the development of strategies for the facile and high-throughput creation of multilayered and multimaterial tissue-like constructs. Real tissues are composed by multiple micrometer-thickness layers of distinct cell types. Although appealing, the cost-effective fabrication of multi-material, and perhaps multi-cell type, lamellar microarchitectures has proven to be challenging, especially when adjacent thin, perhaps single cell layers, of multiple cell types are desired. The current bioprinting and bioassembly technologies are capable of fabricating relatively complex lamellar architectures, but have difficulty placing large surfaces of different types of cells next to each other in a cost-effective manner. For instance, current strategies for multi-material bioprinting or bioassembly of multiple inks in the same printing operation face severe limitations in resolution and speed. A combination of multiple channels, each one dispensing one material, has been demonstrated to fabricate multi-material constructs with resolutions in the range of 50-100 micrometers. Multi-material 3D printing of perusable multi-layered cannulas by co-extruding multiple streams of inks through a set of concentric capillary tubes contained in a single nozzle has also been demonstrated. Similarly, extrusion printing techniques that produced multi-material tissue-like microstructures by co-extrusion of different materials through a head with a pre-set internal architecture have also been shown. These approaches to 3D bioprinting produce structures at resolutions dictated (in the best scenario) by the smallest relevant length scale of the nozzle (i.e., 300-500 μm) and exhibit only moderate speeds (i.e., BioX from Cellink prints at a maximum linear speed of the printhead of 40 mm/s). To date, no study has demonstrated the robust, fast, and cost-effective fabrication of reproducible micro- and nanostructures in a multi-material construct through a single-nozzle printhead.

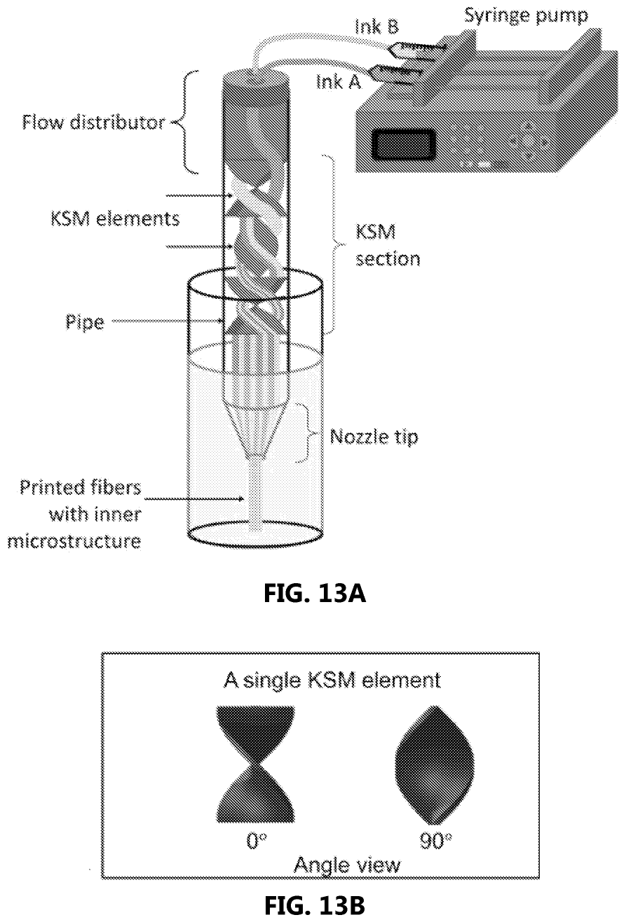

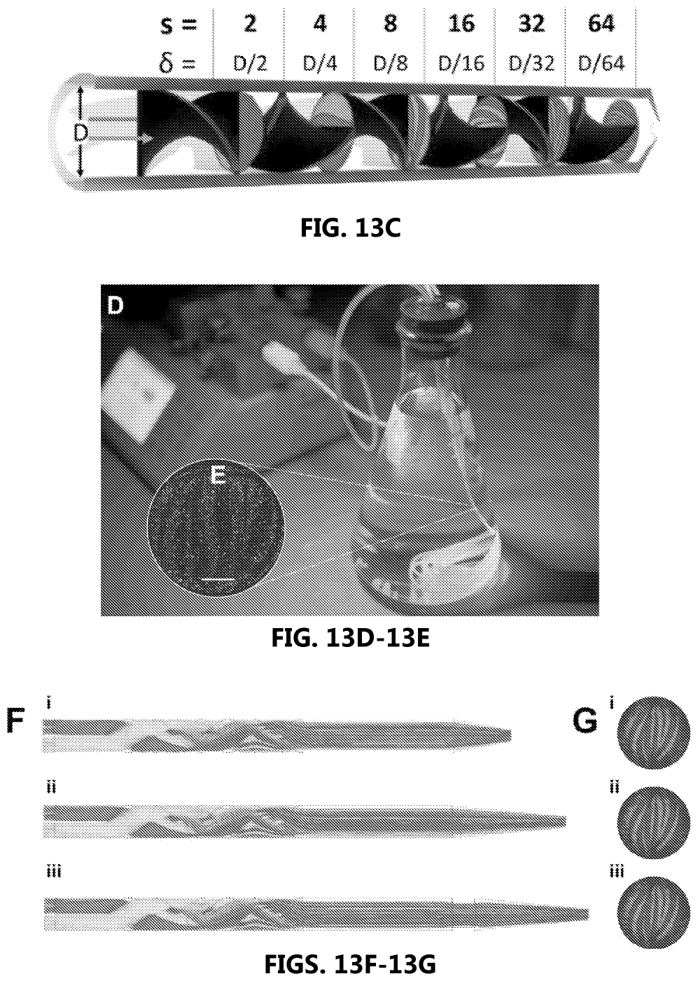

Here, we introduce the concept of continuous chaotic printing: the use of a simple laminar chaotic flow induced by a static mixer for the continuous creation of fine and complex structures at the micrometer and submicrometer levels within polymer fibers. Chaotic flows are used to mix in the laminar regime, where the conditions of low speed and high viscosity preclude the use of turbulence to achieve homogeneity. In the context of 3D printing, they have been suggested as a tool to provide better homogenization of different materials. However, a much less exploited characteristic of chaotic flows is their potential to create defined multi-material and multi-lamellar structures. In Example 1, we demonstrated the use of simple chaotic flows (i.e., Journal Bearing flow) to imprint fine microstructures within constructs in a controlled and predictable manner at an exponentially fast rate in a batch-wise fashion. In this Example, we explore the utility of a chaotic printer, equipped with a Kenics static mixer (KSM) as a key component of the printhead, for the printing of alginate-based fibers with massive amounts of lamellar microstructures in a continuous fashion (FIGS. 13A-13G).

Results and Discussion

Continuous chaotic printing: A simple and effective microfabrication strategy. Our chaotic printer is composed of a flow distributor, a pipe, a static mixing section, and an outlet or nozzle tip (FIGS. 13A-13B). The number of inks one can use is unrestricted, and different distributor geometries can be employed to accommodate the injection of multiple inks. However, for purposes of proof-of-principle studies, we elected to evaluate the simplest printing scenarios. To this end, we adopted a distributor configuration (FIGS. 13A, 13C) for dispensing two inks in a symmetrical fashion. The mixing section contains a KSM, a static mixer configuration that includes a serial arrangement of n number of helical elements contained in a tubular pipe, with each element rotated 90° with respect to the previous one (FIGS. 13B, 13C). In the laminar regime, the KSM (and other static mixers) produces chaos by repeatedly splitting and reorienting materials as they flow through each element. With this simple mechanism, lamellar interfaces are effectively produced between fluids (e.g., printheads containing 1, 2, 3, 4, 5, or 6 KSM elements will produce 2, 4, 8, 16, 32, or 64 defined striations; FIG. 13C). Our results show that multi-material lamellar structures with different degrees of inter-material surface can be printed using a single nozzle by simply co-extruding two different materials (i.e., inks) through a KSM.

In this Example, we used sodium alginate to formulate different inks including a pristine alginate or suspensions of particles (polymer microparticles, graphite microparticles, mammalian cells or bacteria). For instance, we conducted experiments in which one or two types of fluorescent microparticles (e.g., red and green bacteria or red and green polymer beads) were injected into the ports of the mixer distributor (FIGS. 13D, 13E). The result is continuous composite fibers with complex lamellar microstructures (FIG. 13E) that can be stabilized simply by crosslinking in a bath of calcium chloride solution. This preserved the internal microstructure of the fibers with high fidelity. Fine and well-aligned microstructures with defined features can be robustly fabricated along the printed fibers at remarkably high extrusion speeds (1-5 m of fiber/min). Further, a vast amount of contact area is developed within each linear meter of these fibers. This printing strategy is also robust across a wide range of operation settings. We conducted a series of printing experiments at different inlet flow rates to assess the stability of the printing process. As long as the flow regime is laminar and the fluid behaves in a Newtonian manner, the quality of the printing process is not affected by the flow rate used in a wide range of flow conditions. For example, using a cone-shaped nozzle-tip with an outlet diameter of 1 mm, stable fibers were obtained in a window of flow rates from 0.003 to 5.0 mL min-1 (FIG. 13D). Having printheads with different geometries (different degrees of slope) did not disturb the lamellar structure generated by chaotic printing. CFD simulation results suggested that the angle of inclination of the conical tip of the printhead (nozzle tip) did not affect the microstructure within the fiber in the range of the tested flow rates and reduction slopes.

FIGS. 13F and 13G show a computational analysis of the effect of the shape of the printhead tip (angle) on the conservation of the microstructure of printed fibers produced from a mixture of alginate inks containing red and green particles.

Multilayered and well-aligned microstructures. The fabrication of fibers with fine lamellar microstructures will enable the design of materials for relevant biological applications such as the development of high surface biosensors, or composite materials with tunable mechanical properties for cell culture or bio-actuation.

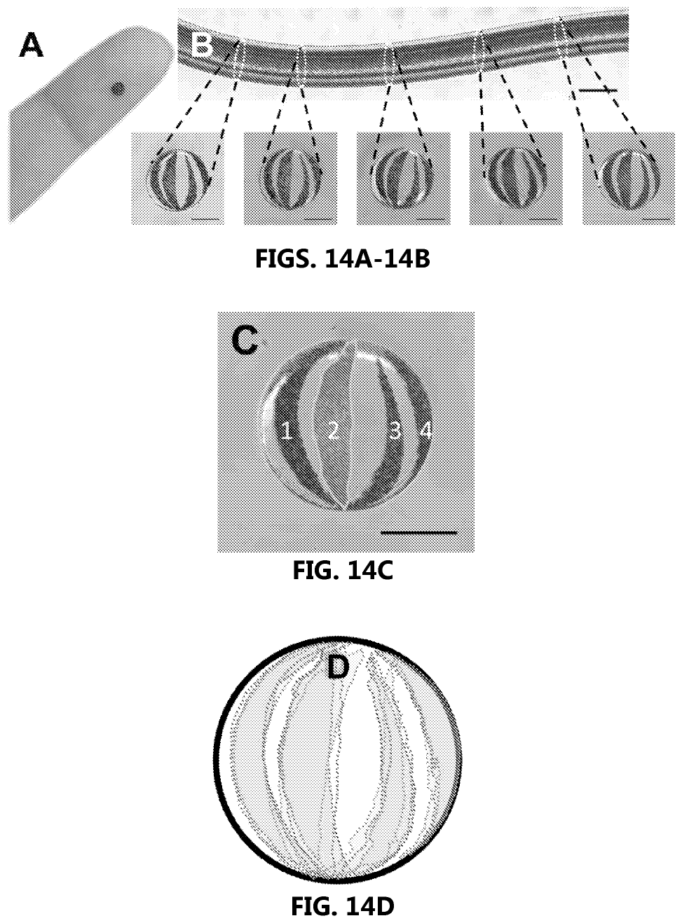

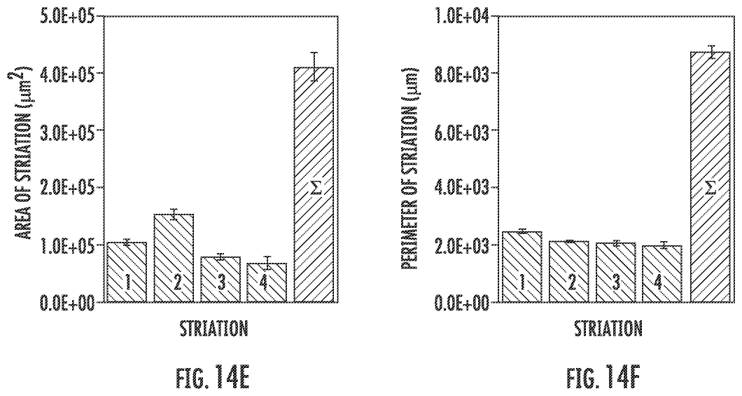

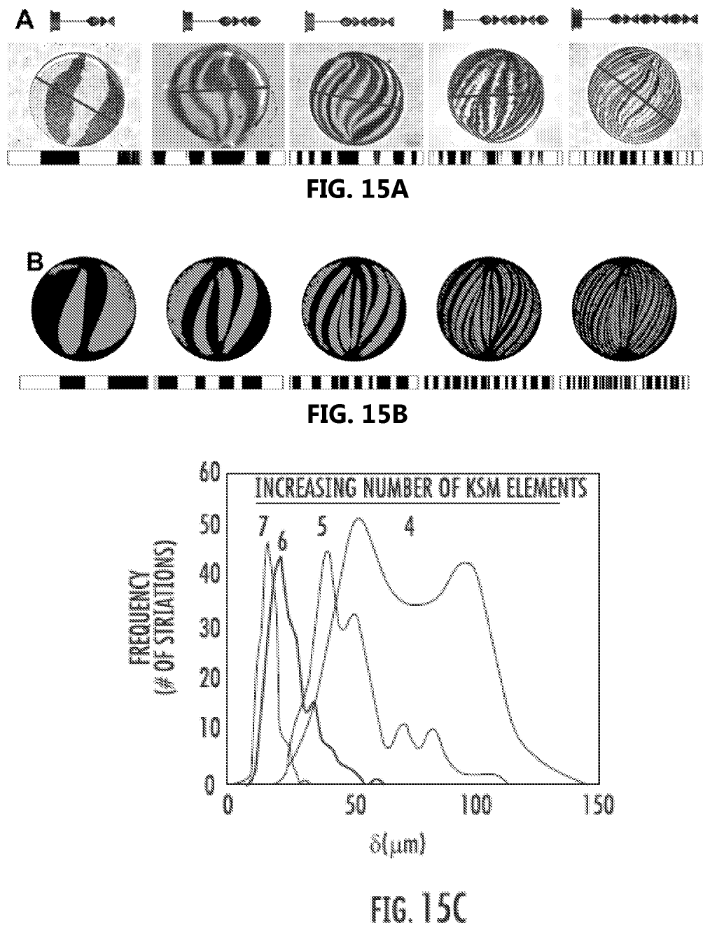

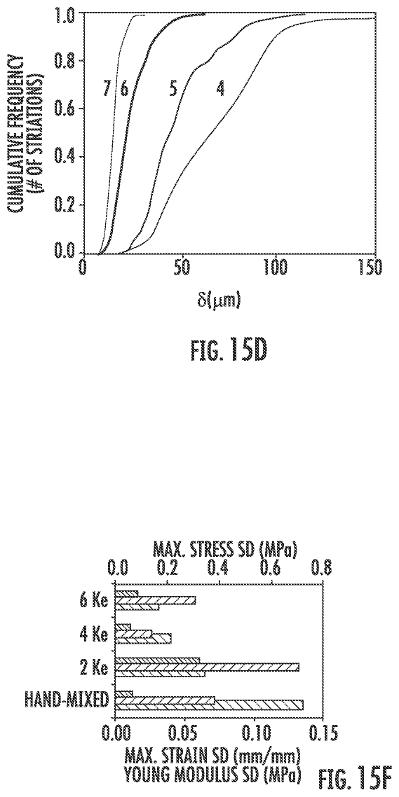

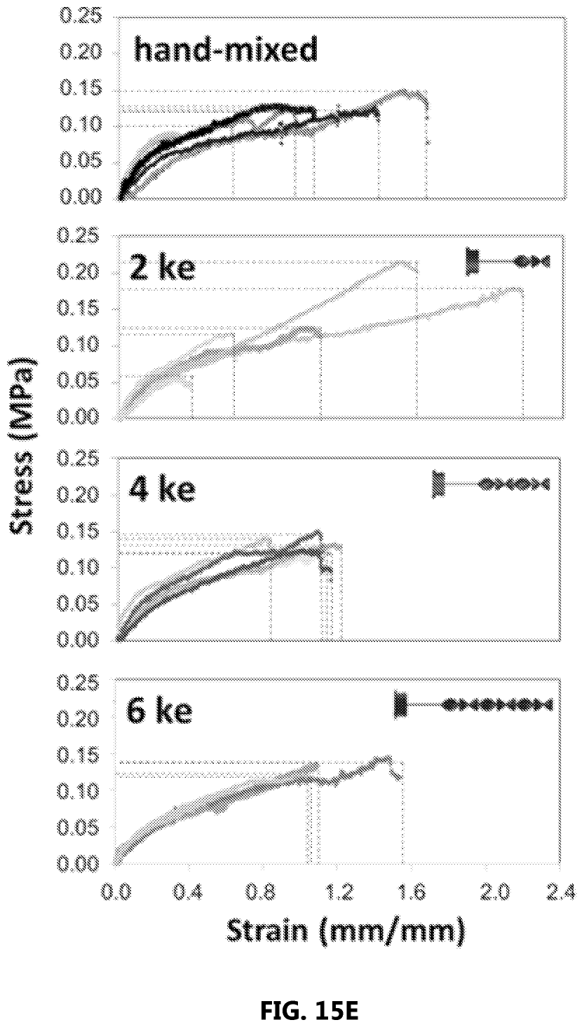

In FIGS. 14A-14F and 15A-15F, we present the results of an experiment in which a suspension of 0.5% graphite microparticles in pristine alginate ink (1%) was co-extruded with pristine alginate ink (1%). For this illustrative experiment, the printhead outlet had a diameter of 1 mm (FIG. 14A). Note that the features in the extruded structure were remarkably similar at different lengths of the fiber (FIGS. 14B-14F). For instance, we calculated the area (shadowed) and the perimeter (indicated with a highlighted line) of each of the graphite striations in five cross-sectional cuts along a fiber segment (FIG. 14C). FIG. 14D shows an overlap of the microstructure for three of these cross-sections. The standard deviation of the area (FIG. 14E) and perimeter (FIG. 14F) for each of the striations is relatively small (the variance coefficient smaller than 10%). This illustrates the robustness of this printing strategy with small nozzle diameters, as well as the reproducibility of the microstructure obtained at different lengths of the fiber. Notably, the resolution of this technique is controlled by both the diameter of the nozzle and the number of mixing elements. As the number of elements used to print increased, the number of lamellae observed in any given cross-sectional plane of the fiber also increased, while the thickness of each lamella decreased (FIG. 15A). Therefore, users of continuous chaotic printing will have more degrees of freedom to determine the multi-scale resolution of a construct, as this is no longer mainly restricted by the diameter of the nozzle (or the smallest length-scale of the nozzle at cross-section). For instance, for our two-stream system (FIG. 13C), the number of lamellae increases exponentially according to the simple model s=2n, where s is the number of lamellae or striations within the construct and n is the number of KSM elements within the extrusion tube.

Two streams of inks co-injected into the printhead will generate 4, 8, 16, 32, and 64 distinctive streams of fluid when passing through a series of 2, 3, 4, 5, and 6 KSM elements, respectively (FIG. 15A). The average resolution of the structure will then be governed by the average striation of the construct (δ), given by δ=D/s, were D is the nozzle inner diameter (FIG. 13C). Since stretching is exponential in chaotic flows, the reduction in the length scale is also exponential, as is the increase in resolution (i.e., more closely packed lines). In the experiment portrayed in FIGS. 15A-15F, the cross-sectional diameter of the fibers was 2 mm. We observed defined average striations with resolutions of ˜500, 250, 125, 62.5, and 31.75 μm by continuously printing using 2, 3, 4, 5, and 6 KSM elements, respectively. Even when 6 KSM elements were used, distinctive lamellae could be discriminated in the array of 64 aligned striations (FIG. 15A). The resolution values obtained through 6 elements already exceeded those achievable by state-of-the-art commercial 3D extrusion printers (˜100-75 μm) that use hydrogel-based inks (e.g., commercial bioprinters).

Another remarkable characteristic of continuous chaotic printing is that the structure obtained is fully predictable, since chaotic flows are deterministic systems (as in any chaotic system). Simulation results, obtained by solving the Navier-Stokes equations of fluid motion using computational fluid dynamics (CFD), closely reproduced the cross-sectional lamellar microarchitecture within the fibers (FIG. 13F, 13G; FIG. 15B).

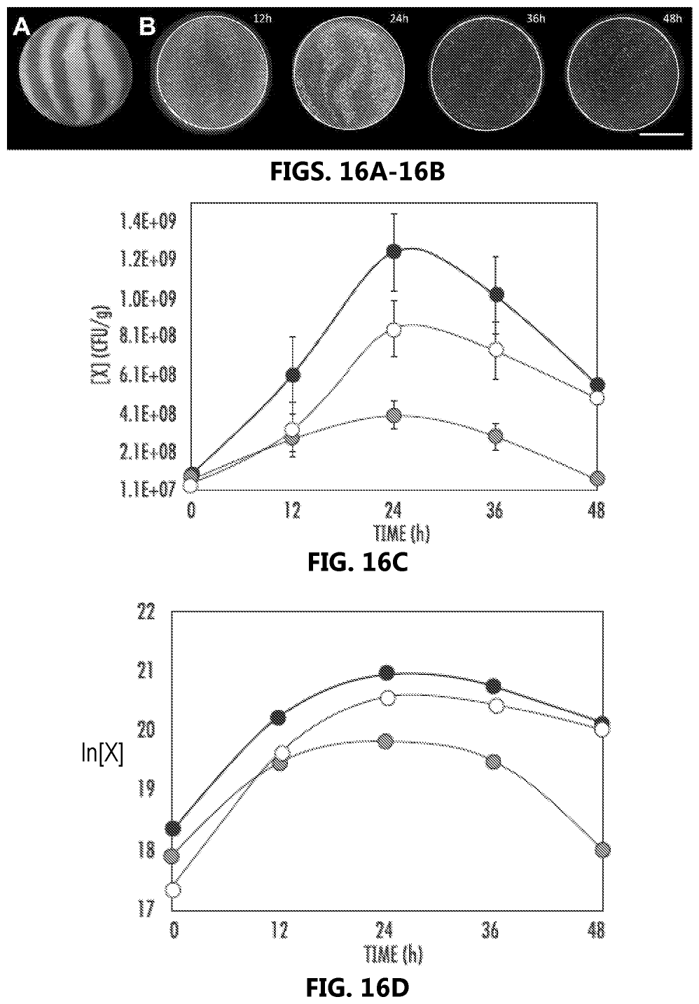

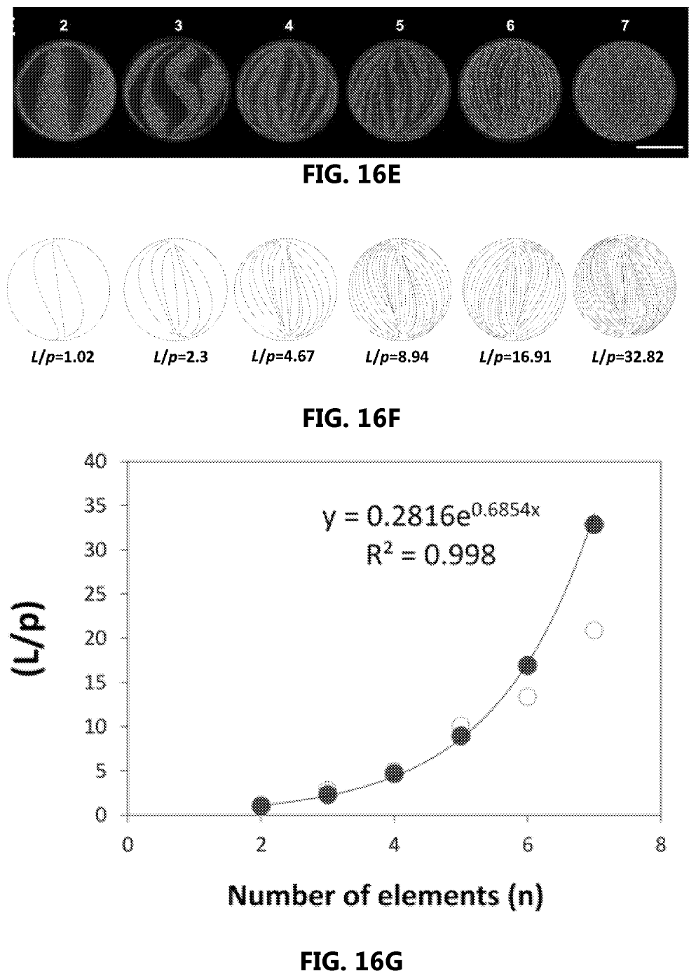

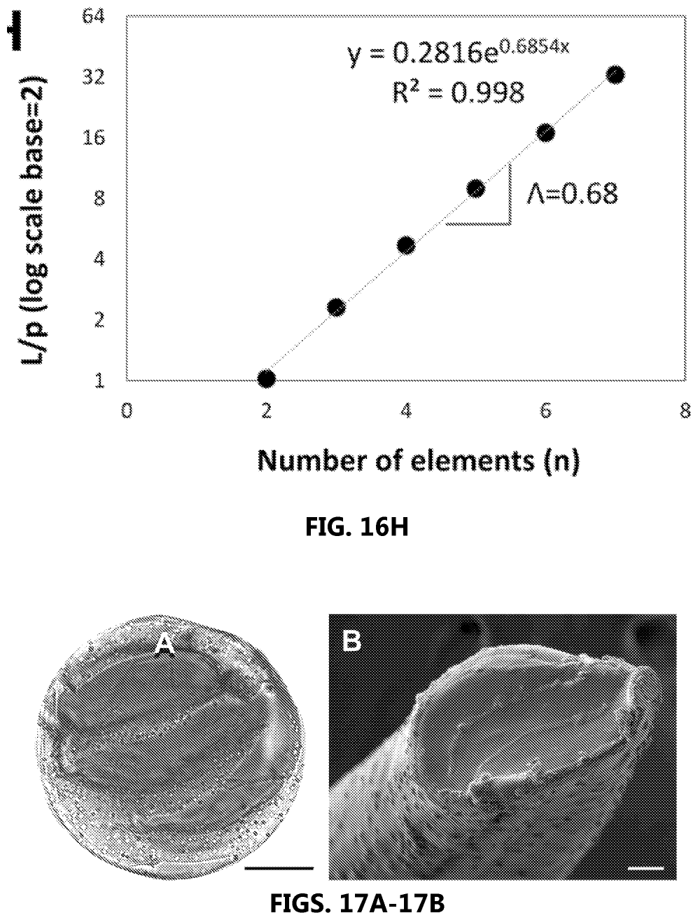

Moreover, we used optical microscopy and image analysis techniques to characterize the fine array of lamellae experimentally produced by continuous chaotic printing. We calculated the striation thickness distribution (STD) on the cross-sections of the graphite/alginate fibers. We did this by drawing several center lines of representative cross-sections and then calculating the distance between striations along those lines. The frequency distribution and the cumulative STD were then measured. FIGS. 15C and 15D, respectively, show the STD and the cumulative STD for constructs printed using 4, 5, 6, and 7 KSM elements. Remarkably, this family of distributions exhibits self-similarity, one of the distinctive features of chaotic processes. As discussed, for any of these particular cases, the average striation thickness could be calculated as the fiber diameter/number of striations. However, due to the highly skewed shape of the distribution toward smaller striation thicknesses (FIG. 15D), the median striation thickness is lower than the average striation value. For example, for the case where 4 KSM elements were used, the average striation thickness can be calculated as 2 mm/16=125 μm. Indeed, 50% of the striations measured less than 125 μm (FIG. 15C, 15D), but the corresponding STD showed that most of the striations had a median value of about 75 μm. This has profound implications for crucial processes such as cell attachment, cell signaling, local reaction kinetics, and mass and heat transfer. For instance, the diffusional distances (δ) in these constructs decreased rapidly with an increase in the number of elements used to print (i.e., following the model δ/(2n)). The diffusional length scales are then reduced by half each time that a KSM element is added to a chaotic printhead. Since diffusion time increases with the square of the diffusion distance (and is only inversely proportional to the diffusion coefficient), the diffusion time decreases 8-fold per element added. This implies that the time relevant to cell signaling decreases 8-fold (almost an order of magnitude) if one KSM element is added to the printhead. As discussed below, the intermaterial area per unit of volume, which is key for surface-catalyzed reactions and cell attachment, increases exponentially as the number of elements is increased (FIGS. 16A-16H).

Fiber and particle alignment is important in many applications in materials technology. We next show that the fabrication of well-aligned microstructures achievable through chaotic printing can influence relevant characteristics of composites, such as the robustness of their mechanical performance. We characterized the mechanical properties of alginate-based fibers 2.5 cm in length produced by chaotic printing (and therefore having different internal structures) or hand-mixing and extrusion through an empty pipe (FIG. 15A-15F). Specifically, we conducted tensile testing using a universal testing machine on fibers produced from a mixture of 0.5% graphite microparticles in alginate, generated either by hand-mixing (a control without lamellar structures) or by continuous chaotic printing using 2, 4, or 6 KSM elements. FIG. 15E shows the stress-strain curves associated with the resulting fibers. We did not find significant differences in the Young's modulus, ultimate stress, or maximum elongation at break in these sets of fibers. However, the fibers exhibited less variability when produced by continuous chaotic printing than those by extrusion of hand-mixed inks. Among the fibers produced by chaotic printing, the fibers were more homogeneous when co-extruded through printheads containing 4 or 6 elements than only 2 elements. FIG. 15F shows an analysis of the standard deviation of relevant mechanical performance indicators associated with different microstructures. These results suggest that effective alignment of the microstructures within the fibers resulted in a more reproducible mechanical performance in structurally complex materials such as alginate hydrogels.

Bioprinting applications. Bioprinting (i.e., the printing of living cells and biomaterials in a predefined fashion) is presently even more limited in resolution and speed than additive manufacturing techniques in general. We further illustrate a biological application of continuous chaotic printing by fabricating constructs with specific microarchitectures containing living cells.

Tightly controlling the degree of intimacy (e.g., the density of interfaces between bacterial populations) may enable the fabrication of 3D multi-material constructs with novel functionalities and is of paramount importance in modern microbiology, for example on the design of physiologically relevant gut-microbiota models. The spatial arrangement and distribution of bacteria, recently described as “microbiogeography”, is an important determinant of bacterial community dynamics. Different species of bacteria interact with other microorganisms through chemical signals. For example, quorum sensing, a well-studied phenomenon, depends on the vicinity and the amount of surface area shared among bacterial communities. In general, the dynamics of competition or mutualism in mixed microbial communities is strongly influenced by spatial distribution. However, relatively few studies have addressed the relationship between spatial distribution, distance, and cell density in bacterial systems. This is partially due to the fact that conventional microbiology techniques offer only a limited degree of control over the spatial organization of mixed cultures.

Continuous chaotic printing enables precise control of the spatial distribution of bacterial communities, aligned in a micro-lamellar microstructure, and allows meticulous and unprecedented design and regulation of the amount of interface between bands of bacteria. In FIGS. 16A-16E, we used two recombinant Escherichia coli strains, one producing red fluorescent protein (RFP) and the other producing green fluorescent protein (GFP) to fabricate cell-laden fibers. Well-defined bacterial striations could be printed by our technique (FIG. 16A).

Remarkably, the bacteria could be cultured in these fibers for extended time periods. We followed the kinetic behavior of both bacterial populations (i.e., GFP- and RFP-bacteria) in the fibers initially seeded at low concentrations. During the first 24 h of culture (from t=0 to 24 h), the intensity of the fluorescence produced by the bacterial colonies increased, while the bacteria continued to respect the original patterns in which they had been printed (FIG. 16B). We corroborated the increase in the number of live bacteria by conventional colony-forming units (CFU) microbiological assays (FIGS. 16C, 16D). To do this, we sampled multiple sections of fibers. We consistently observed that the bacterial populations grew in both areas for the first 24 h, exhibited a short plateau, and then later decayed. Interestingly, we observed a statistically significant difference in the number of viable green and red bacteria (P<0.05) at 24 and 48 hours after printing. These differences suggest that these two populations of bacteria, although practically identical in their genetic makeup, establish a competition for resources along shared interfaces. These results demonstrated that chaotic printing can be used for the fabrication of dynamic living systems that are capable of evolving in time from very well-defined initial conditions. In addition, massive amounts of interface between green and red bacterial regions could be developed if more elements were used during printing. For example, the boundary between the green and red bacterial regions can be effectively tuned from ˜1 mm down to 15 μm by varying the number of KSM elements used to print (from 2 to 7, FIG. 16E). Since the maximum length of these bacteria is ˜2 μm, we were able to imprint lamellae of bacteria in the resolution range of tens of micrometers. The diameter of these fibers was 1 mm. Therefore, printing using 7 KSM elements yielded lamellae with average striation thicknesses of less than 10 μm (median lower than 7 μm). This means that each lamella might accommodate a few bacterial cells across its width. While a characteristic standard deviation occurs with chaotically printed constructs (FIG. 15E), the structures obtained by chaotic printing are repeatable. This will translate into the fact that the “overall” functionality of the construct (i.e., the bacterial community or tissue construct to be fabricated) will be dictated by the architecture. Please note that, in FIG. 15C, the STDs of constructs printed using 4 and 6 (or 5 and 7) elements are distinguishable (i.e., their overlapping is minimal).

In chaotic printing, the amount of inter-material area fabricated increases exponentially as a function of the number of elements used. We used image analysis techniques to quantify, at high magnification, the shared perimeters between red and black lamellae in FIG. 16E (cross-sectional cuts). Indeed, the amount of black-red perimeter at cross-sections grew exponentially with the increasing number of elements (FIGS. 16E, 16G, and 16H).

In addition, using computational strategies, we simulated the amount of surface area generated by the printing process in constructs printed using different KSM elements (FIG. 16F), and confirmed the data obtained experimentally (FIGS. 16E, 16G). For instance, when 6 elements were used to print, approximately 15 cm of shared linear interface were developed between the two materials (inks) at each cross-sectional plane (D=0.1 cm); the ratio between the total amount of developed interface and the fiber perimeter was 16.91. This created a remarkably high density of shared interface (6.76 cm/mm²). Since the fiber exhibits the very same microstructure along its entire length (FIG. 15B), the inter-material surface density generated inside the fiber could be determined as 0.067 m² cm⁻³.

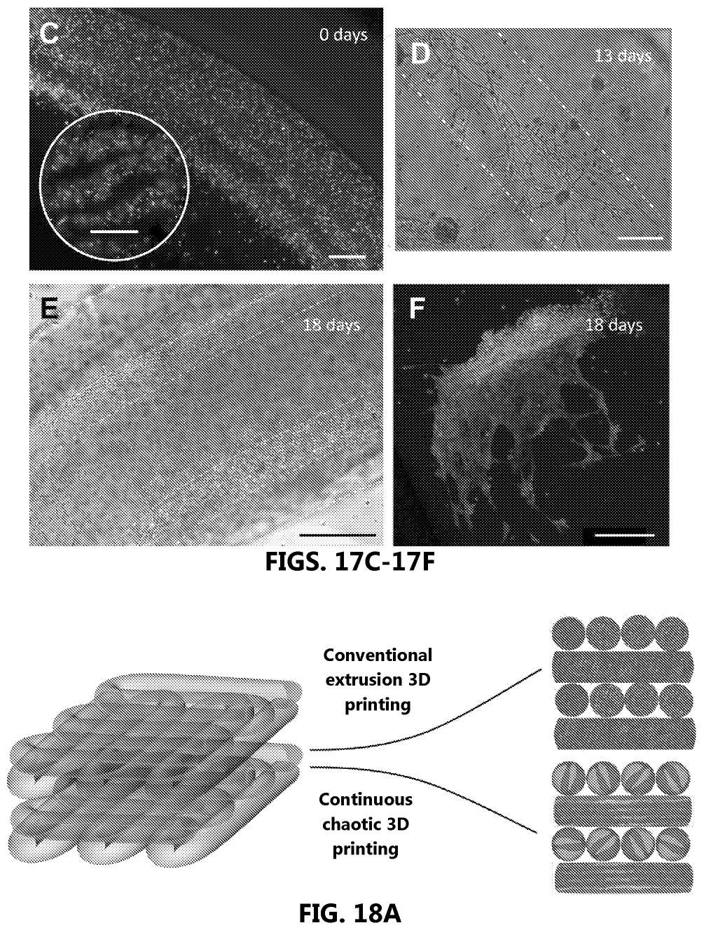

In tissue engineering scenarios, multi-material and multilayer structures can be used to mimic the architecture and functionality of real tissues. We also conducted chaotic bioprinting experiments in which we fabricated bands of C2C12 murine skeletal myoblasts within alginate fibers added with GelMA. We present the cross-sectional (FIG. 17A) and longitudinal view (FIG. 17B) of a cell-laden alginate fiber lightly enriched with protein (GelMA) to favor cell attachment and eventual proliferation.

Well-defined bands of C2C12 cells are distinguished along the hydrogel fibers. Mammalian cells are shear-sensitive; however, the low shear laminar conditions prevalent at the printhead tip enabled high initial cell viabilities (higher than 90%; FIG. 17C). Cells survived and proliferated within these fibers. Most cells remained within the striations corresponding to the cell-laden ink. After 7 days of culture, the cells began to spread and interact within each other, and some clusters of cells appear. After 2 weeks of culture (i.e., day 13 and 18), the proliferating cells elongated while maintaining their initial striation patterns (FIGS. 17D and 17E). The fibers were stained to reveal the position of the cell nuclei and their cytoskeletons. Note that in some cell clusters, multinucleated cells, a signature of myotubule development, started to be evident (FIG. 17F). This illustrative experiment suggests the potential of chaotic bioprinting to produce living fibers that closely resemble the multilayered structure observed in mammalian tissues and display massive amount of material interface. As demonstrated above, using a chaotic printhead containing 6 KSM elements, 0.067 m² of material interface could be accommodated per 1 cm³ of cell-laden fiber. Therefore, ˜67 m² L⁻¹ of well-aligned inter-material surfaces could be fabricated within these living constructs. For comparison, human kidneys have an approximate volume of 150 cm³, and the total area of the capillaries of all the glomeruli within them is 0.6 m² (4 m² L⁻¹). Printing at the flow rate of 1 mL min⁻¹, which is a typical printing flow rate used in our system, could generate this amount of area per unit of volume every minute.

This massive amount of interface cannot be fabricated at this speed, precision, or resolution by any of the currently available micro-fabrication or printing platforms. It should be noted that flow rates of up to 3 mL min⁻¹ could be conveniently achieved using our printing method.

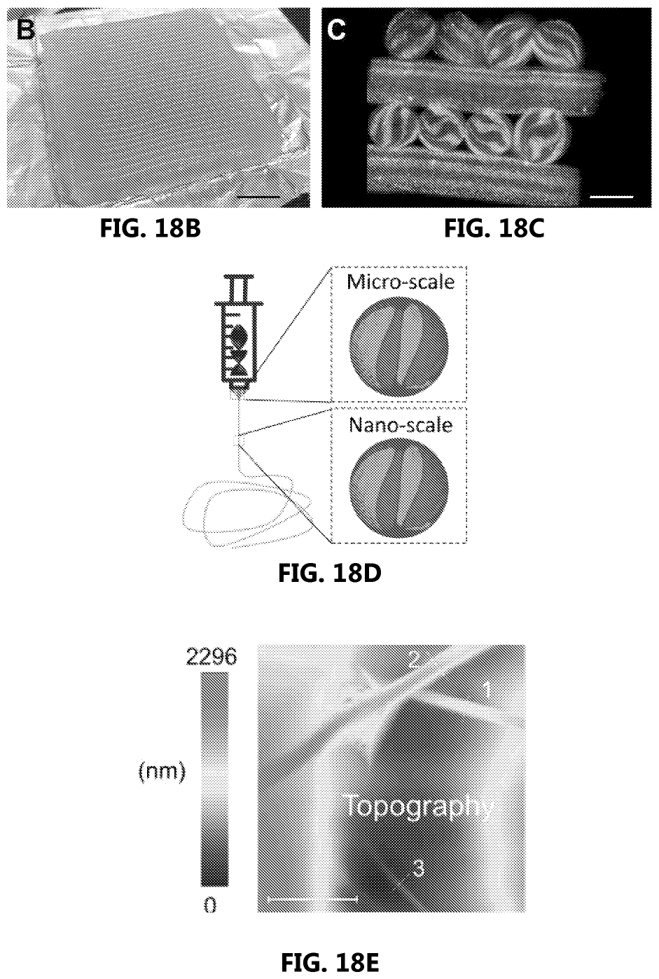

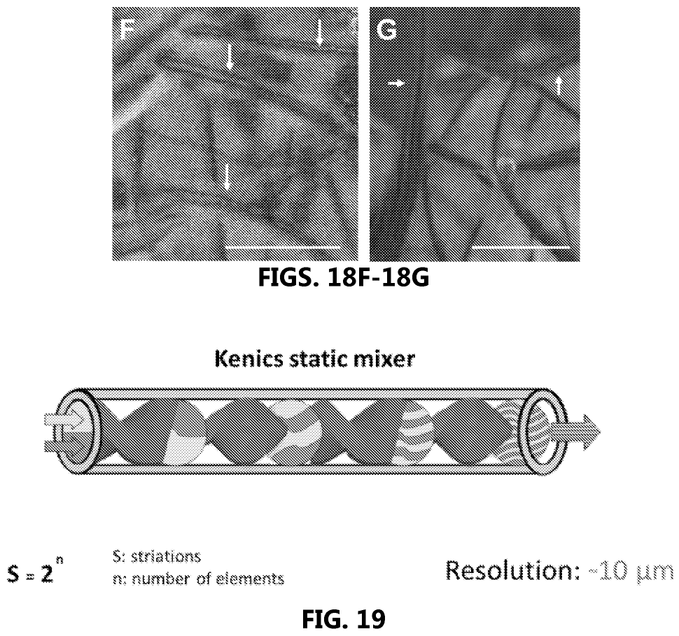

Coupling of continuous chaotic printing with other fabrication techniques. The combination of continuous chaotic printing with other fabrication technologies (e.g., molding, electrospinning, or robotic assembly) can provide access to complex multi-scale architectures with high degrees of predictable external shapes and internal microstructure. Indeed, during printing, these fibers can be rearranged either into macrostructures or the individual fibers can be further reduced in diameter while preserving their lamellar architecture (FIGS. 13F, 13G: FIGS. 18A-18C). We illustrate this by printing a long fiber of alginate containing multiple lamellae and then rearranging it into a block of several layers of fiber segments (FIGS. 13A-13C). The integration of this multi-material printhead into a 3D printer may thus enable rapid fabrication of multi-material (and/or multi-cellular) constructs that exhibit a great amount of material interface with a complex and tunable hierarchical architecture.

Also, chaotic printing may be coupled with other techniques for the production of nanofibers that contain finely controlled structures at the submicron scale (FIGS. 18D-18G). This may enable, for example, the fabrication of microsensors or microactuators with enormous surface area, for biological applications. As an example, we coupled a 2-element KSM printhead with an electrospinning device (FIG. 18D) to produce a mesh of nanofibers containing well-defined nanostructures. Fibers produced by 3D chaotic printing were continuously solidified as they were generated by direct feeding into an electrospinning apparatus, further reducing the fiber mean diameter to <300 nm (FIG. 18E). In this hybrid fabrication strategy, fiber solidification occurs by rapid evaporation during electrospinning, instead of crosslinking by immersion in calcium chloride.

Remarkably, the structure produced by chaotic printing is preserved during electrospinning. Based on estimates of the shape of the Taylor cone (˜0.18 μL) and the rate of injection of the materials (2-5 μL min⁻¹), the average residence time in the Taylor cone is ˜2-5 s. The diffusion time may be roughly calculated as δ2/D, where δ is the average striation thickness (δ=0.0128 when 3 KSM elements are used), and D is the diffusion coefficient. The diffusion coefficient of relatively small organic molecules in PEO has been reported as ˜108. Therefore, the actual diffusion coefficient of PEO in alginate should be much lower, at ˜109. The diffusion time for this process should then be in the range of ˜1000 s, or much higher than the residence time. Since the residence time at the Taylor cone is about 3 orders of magnitude shorter than the diffusion time, electrospinning should have a negligible effect on the structure obtained by 3D chaotic printing.

A close inspection using photo-induced force microscopy (PIFM) revealed multilayered nanostructures with average striation thicknesses in the range of 75-100 nm (FIGS. 18F, 18G). These results demonstrated that the microstructure created by 3D chaotic printing can be further scaled down by three orders of magnitude using electrospinning.

Conclusion

In this Example, we have described continuous chaotic printing as a strategy that allows for control of the spatial microstructures (e.g., number of layers and average spacing between them) within a single 3D printed fiber. This technological platform uses an on-line static mixer in a printhead to provide a partial mixing of different materials as they are coextruded through the nozzle tip. In particular, we have adopted the KSM as the first model and have used it to fabricate, in a simple fashion, highly convoluted 3D structures within polymer composites in a continuous stream at high speeds (>1.0 meters of fiber/min). The diameter of the printing head and the number of mixing elements determine the number and thickness of internal lamellae produced according to a process of successive bifurcations that yields an exponential generation of inter-material area. Illustratively, by using 6 internal elements, 64 lamellae of average widths of 15 μm can be generated in a 1 mm cross-section fiber, and an inter-material area of ˜67 m² L⁻¹ can be achieved. These values for microstructure resolution, internal surface area density, and fabrication speed all exceed the capabilities of any of the currently available commercial microfabrication techniques (i.e., commercial 3D printers) for the creation of microstructure.

Our results demonstrate the unrivaled ability of chaotic printing to deploy cells within high Surface Area to Volume (SAV) fibers. As available bioprinting and bioassembly technologies approach the resolution and SAV of chaotic printing, they also tend to require long fabrication times and mechatronically coordinated control systems. In addition to multicellular, high SAV constructs, chaotic printing offers other breakthroughs in regards to currently available multi-material printing technologies that, typically, require optimized inks that must be deployed under a specific and narrow range of conditions.

The fundamentals underlying chaotic printing are solid, as this type of printing relies on the use of chaotic flows to develop microstructure at an exponential rate in a deterministic manner. Indeed, we have shown that the microstructure resulting from the use of different numbers of KSM elements is amenable to rigorous modeling using CFD simulations, and the resemblance between our experimental and simulation results is remarkable. This precise predictability of the microstructures within a printed construct will greatly expand the application of 3D printing and the complexity of printed composites. A wide spectrum of microstructures can be designed and obtained using this technique. The adoption of different types of static mixer elements (i.e., SMX, Sultzer, and novel ad hoc designs), the use of more than two inks (or materials), the manipulation of the injection location, and the dynamic changes in the speed of each one of the injections, can open up possibilities for obtaining structures with various degrees of complexity in a wide range of scales. For instance, we showed that chaotic printing is a simple and versatile micro- and nano-fabrication platform and that, when coupled with other fabrication resources, can generate macro-structures with an enormous amount of interface between their constituent materials.

The fabrication method introduced here produces non-uniform, but reproducible and well-defined, lamellar patterns. Chaotic printing adds to the existing arsenal of tools currently available to the biofabrication community. Reproducible non-uniformity is precisely one of the strengths of this printing method (and a signature of Nature). We were bioinspired by some of the highly complex (and ordered) patterns that are produced in Nature, which are mostly non-uniform (i.e., vasculature, marble and other multilayered geological formations, multi-layered tissues, and real bacterial communities, among many others).

Remarkably, our variability is statistically robust. As we have shown (FIGS. 15E, 15F), the striation thickness distribution (STD) of the microstructure generated through chaotic printing is known, reproducible, can be calculated by simulations, and is even self-similar (meaning that the overall shape of the STDs obtained by printing with different numbers of elements closely resemble each other). All these properties are rooted in the fundamental physics of chaotic advection. Chaotic flows generate microstructure with a reproducible distribution of length scales.

In an exciting further development, we demonstrated that the output of a continuous 3D chaotic printhead can be fed into an electrospinning nozzle to create fiber meshes with lamellar nanostructures. By doing so, we have shown that the microstructure created by 3D chaotic printing can be further scaled down by three orders of magnitude. We envision numerous applications of continuous chaotic printing in biomedicine (i.e., bacterial and mammalian cell bioprinting), electronics (i.e., fabrication of high-sensitivity multi-branch electrodes and supercapacitors), and materials science in general.

Experimental Section

Experimental set-up. Our continuous chaotic printer included a syringe pump loaded with two 10-mL disposable syringes, a cylindrical printhead containing from 2 to 7 KSM elements, and a flask containing 550 mL of 1% calcium chloride (Fermont, Productos Quimicos Monterrey, Monterrey, NL, Mexico) (FIG. 13A). Syringes were loaded with different inks (e.g., particle suspensions in pristine 1% alginate) and connected to one of the two inlet ports located in the lid of the printhead. Details of the geometry of the printer head and the internal KSM elements are shown in FIGS. 13B and 13C. The fabrication of printer heads is described in a following subsection (KSM printheads). The syringe pump was set to operate at a flow rate of 0.8 to 1.5 mL min⁻¹. We conducted experiments using printheads with different internal diameters, in the range from 5.8 to 2 mm. The tube containing the KSM could be connected to a tip to further reduce the diameter of the final fiber. Tip reducers with an outlet diameter of 4, 2, and 1 mm were used in the experiments presented here (FIGS. 13D, 13F, and 13G). The outlet of the tip was submerged in 1% calcium chloride to crosslink the extruded fibers as soon as they exited the tube (FIG. 13D).

KSM printheads. We fabricated our KSM printheads in house. KSM elements were designed using SolidWorks based on the optimum proportions reported in literature. The sets of KSM elements were printed on a P3 Mini Multi Lens 3D printer (EnvisionTEC, Detroit, Mich.) from the ABS Flex White material. We used a length-to-radius ratio of L:3R (FIG. 13B). For example, for printheads with an internal diameter of 5.8 mm, the length and diameter of each separate KSM element were 8.7 mm and 5.8 mm, respectively. Sets of 2, 3, 4, 5, 6, and 7 KSM elements, attached to a tube cap, were fabricated to ensure a correct orientation of the ink inlet ports on the cap with respect to the first KSM (FIG. 13C). The cap was designed so that each ink inlet was positioned on a different side of the first KSM element to maintain similar initial conditions in all experiments (FIG. 13A, 13C).

Printing experiments and ink formulations. We used several different ink formulations for the experiments presented here. Inks consisted of particles suspended in 1% alginate or pristine alginate (CAS 9005-38-3, Sigma Aldrich, St. Louis, Mo., USA) solutions.

In a first set of experiments, we fabricated fibers loaded with either red or green fluorescent particles. Red and green fluorescent inks were prepared by suspending 1 part of commercial fluorescent particles (Fluor Green 5404 or Fluor Hot Pink 5407; Createx Colors; East Granby, Conn., USA) in 9 parts of a 1% aqueous solution of sodium alginate (Sigma Aldrich, St. Louis, Mo., USA). The fluorescent particles were previously subjected to three cycles of washing, centrifugation, and decantation to remove surfactants present in the commercial preparation.

We also used chaotic printing to fabricate fibers containing an overall concentration of 0.5% graphite by co-extruding a suspension of 1.0% graphite in alginate solution (1%) and pristine alginate solution (1%) through printheads containing 2, 4, or 6 KSM elements. In addition, we produced control fibers by extruding pristine alginate (without graphite microparticles) through an empty tube, or by co-extruding two streams of ink containing 0.5% graphite microparticles hand-mixed in alginate.

In a third set of experiments, we used fluorescent inks based on suspensions of fluorescent E. coli bacteria. These fluorescent bacteria were engineered to produce either GFP or RFP. Bacterial inks were prepared by mixing either GFP- or RFP-expressing E. coli in 2% alginate solution supplemented with 2% Luria-Bertani (LB) broth (Sigma Aldrich, St. Louis, Mo., USA). For ink preparation, bacterial strains were cultivated for 48 h at 37° C. in LB media. Bacterial pellets, recovered by centrifugation, were washed and re-suspended twice in alginate-LB medium. The optical density of the re-suspended pellets was adjusted to 0.1 absorbance units before printing (approximately 5×10⁸ colony forming units per mL (CFU mL⁻¹)). Fibers were printed at a flow rate of 1.5 mL/min and cultured by immersion in LB media for 72 hours. The number of viable cells present in the fibers at different times was determined by conventional plate-counting methods. Briefly, fiber samples of 0.1 g were cultured in tubes containing LB media. The number of viable cells was determined by washing the 0.1 g samples in 1× phosphate-buffered saline (PBS) at pH 7.4 (Gibco, Life Technologies, Carlsbad, Calif.) to remove the bacteria accumulated in the LB media. Each sample was disaggregated and homogenized in 0.9 mL of PBS. The resultant bacterial suspensions were decimally diluted, seeded onto 1.5% LB-Agar (Sigma Aldrich, St. Louis, Mo., USA) plates, and incubated at 37° C. for 36 h.

We also bioprinted muscular murine cells (C2C12 cell line, ATCC CRL 1772) in 1% alginate inks supplemented with 3% gelatin methacryloyl (GelMA) added with a photoinitiator (0.01% LAP). To this purpose, a first ink contained only alginate and GelMA, while the second was cell-laden with C2C12 cells at a concentration of 3×106 cell mL⁻¹. Cell laden fibers, obtained by immersion in alginate and then further crosslinked by exposure to UV light at λ=400 nm for 30 s. The bioprinted and cell-laden fibers were immersed in DEMEM culture medium (Gibco, USA) and incubated for 20 days at 37° C. in an 5% CO₂ atmosphere. Culture medium was renewed every 4th day during the culture period.

In a fifth set of experiments, we produced electrospun nanofiber mats by combining 3D chaotic printing in-line with electrospinning. First, we chaotically printed fibers by coextrusion of a pristine alginate ink (4% sodium alginate in water) and polyethylene oxide (7% PEO in water) at a rate of 2-5 μL min⁻¹. The resulting PEO-alginate fibers were then electrospun (in-line) to produce nanofiber mats.

Microscopy characterizations. The microstructure of the fibers produced by chaotic printing was analyzed by optical microscopy using an Axio Imager M2 microscope (Zeiss, Germany) equipped with Colibri.2 led illumination and an Apotome.2 system (Zeiss, Germany). Bright-field fluorescence micrographs were used to document the lamellar structures within the longitudinal segments and cross-sections of the fibers. Wide-field images (up to 20 cm²) were created using a stitching algorithm included as part of the microscope software (Axio Imager Software, Zeiss, Germany). Fibers were frozen by sudden immersion in liquid nitrogen to facilitate sectioning while preserving the microstructure. The microstructure of the nanofibers produced by chaotic printing coupled with electrospinning was analyzed by atomic force microscopy (AFM) and photo-induced force microscopy (PIFM), a nano-IR technique.

Mechanical testing of graphite-alginate fibers. We used a universal test bench machine (Tinus Olsen h10kn; PA; USA), with a load cell of 50 N at a rate of 35 mm min⁻¹, to evaluate the mechanical properties of alginate fibers containing 0.5% graphite particles and produced by different printing strategies. Specifically, we conducted tensile testing on fibers produced from a mixture of 0.5% graphite microparticles in alginate, generated either by hand-mixing and extrusion through an empty pipe (a control without lamellar structures) or by continuous chaotic printing using 2, 4, or 6 KSM elements. In these experiments, the gauge length between clamps was set to 25 mm. Stress-strain curves were obtained for each of the five different formulations. We determined the maximum tensile strength, strain at break, and Young modulus of the fibers from stress-strain data.

Computational simulations. The system was simulated using a finite element model (FEM) strategy in COMSOL Multiphysics 5. First, a 3D model was designed and solved, using laminar flow equations and a stationary solver, to determine the velocity field in the system for the various experimental scenarios explored. A fluid viscosity value of 1P and a density of 1000 kg m⁻³ were used. A time dependent solver was then used to track up to 105 massless particles using particle tracking for fluid flow physics in the previously solved stationary velocity field. The simulation was discretized with a reasonable fine mesh composed of free triangular elements. Mesh sensitivity studies were conducted to ensure the consistency of results. No-slip boundary conditions were imposed in the fluid flow simulation, while a freeze boundary condition was employed for the particle tracing module. The interface length was determined by importing the output results from the cross-section of the fibers (a set of points describing the interface position) into CorelDraw software X5 (Corel Corporation, Canada), drawing Bezier curves over the striations, and establishing the length of the curves using the software.

### Example 3. Biofabrication of High Surface/Volume and Perfusable Microstructured Scaffold Using Continuous Chaotic Printing

Utilizing the method of Example 2, perfusable microstructures were prepared using continuous chaotic printing. As in Example 2, the system employs a Kenics static mixer (KSM). An example KSM including 4 KSM elements is illustrated in FIG. 19. As shown in FIG. 19, the KSM induced laminar chaotic flow between a bioink and a fugitive (or sacrificial) ink to produce a microstructured fiber. The bioink phase can then be cured, and the fugitive ink can be removed to produce a perfusable microstructure.

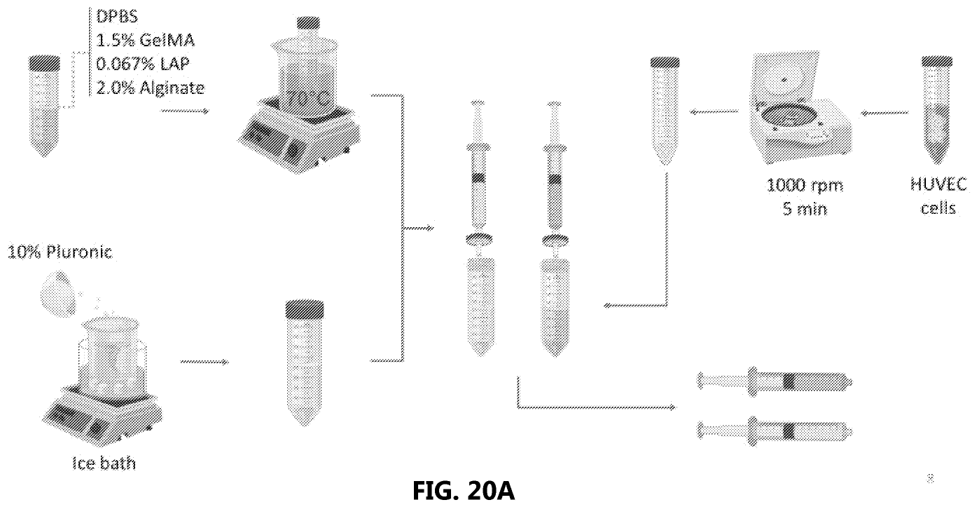

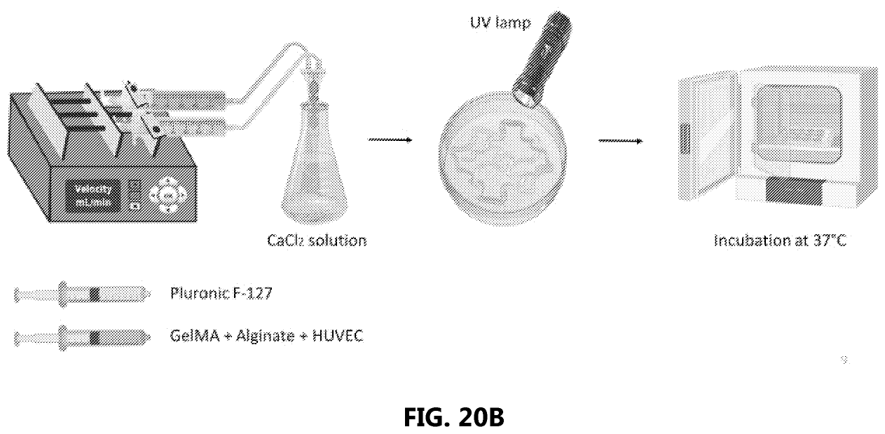

Referring now to FIG. 20A, a bioink was prepared by mixing 1-1.5% by weight GelMA, 2.0% by weight alginate, and 0.067% by weight LAP were dissolved in PBS buffer. Alginate was added after the thermal activation of LAP (Allevi, Philadelphia, USA) at 70° C. The GelMA-containing hydrogel was prepared before each printing process, and it was cooled down at room temperature before coextrusion with P-F127. If desired, cells for culture can be dispersed in the bioink. A bioink may also be prepared using Dulbecco's phosphate-buffered saline (Sigma-Aldrich, St. Louis, USA) to form an aqueous solution of 2% sodium alginate (Sigma-Aldrich, St. Louis, USA). Separately, a fugitive ink was prepared by dissolving a poloxamer (PLURONIC® acid F127; P-F127) in distilled water at concentrations of 10% or 5%. These suspensions were placed in an ice bath under continuous agitation until they became homogeneous solutions. P-F127 hydrogels were stored at 4° C., and they were warmed at room temperature (˜25° C.) before chaotic printing. The bioink and fugitive ink can then be chaotically printed to form a perfusable microstructure as described below.

P-F127 is composed of propylene oxide (PPO) and polyethylene oxide (PEO), forming a PEO-PPO-PEO copolymer. Importantly, P-F127 is a biomaterial approved by the FDA.

The temperature and concentration (% w/v) of P-F127 plays an important role in its rheological properties. For example, an aqueous solution containing 10% P-F127 exhibits a constant viscosity (˜0.1 Pa·s) at a temperature range from 4° C. to 30° C. However, when the concentration of P-F127 is 20%, this hydrogel undergoes a liquid to solid transition at ˜22° C., and its viscosity increases from 200 Pa·s to 1200 Pa·s at 25° C. Therefore, 10% or lower concentrations of P-F127 are suitable for continuous chaotic printing.

Referring now to FIG. 20B, the continuous chaotic printer included a syringe pump loaded with two 10-mL disposable syringes (one containing the GelMA-containing bioink and the other containing the fugitive ink), a cylindrical printhead containing from 3 to 6 KSM elements, and a flask containing a volume of 1% calcium chloride. Details of the geometry of the printer head and the internal KSM elements are shown in FIGS. 13B and 13C and described in Example 2 above. The syringe pump was set to operate at a flow rate of 1.5-3.0 mL min⁻¹. The outlet of the printer head was submerged in 1-2% calcium chloride to crosslink the extruded fibers as soon as they exited the tube. Subsequently, the fibers were exposed to UV light at 365 nm for 30 seconds to cure the GelMA in the fibers. The resulting fibers were then incubated at 37° C. and the fugitive ink was removed.

In the other embodiment, the aqueous solution of 2% alginate was coextruded with 10% P-F127 through a printhead containing a Kenics static mixer (KSM). These hydrogels enabled to print at flow rates ranging from 2.5 to 10 mL/min. Moreover, printheads comprising either 5 or 6 KSM elements allowed to set 1.0 mL/min as the minimum flow rate.

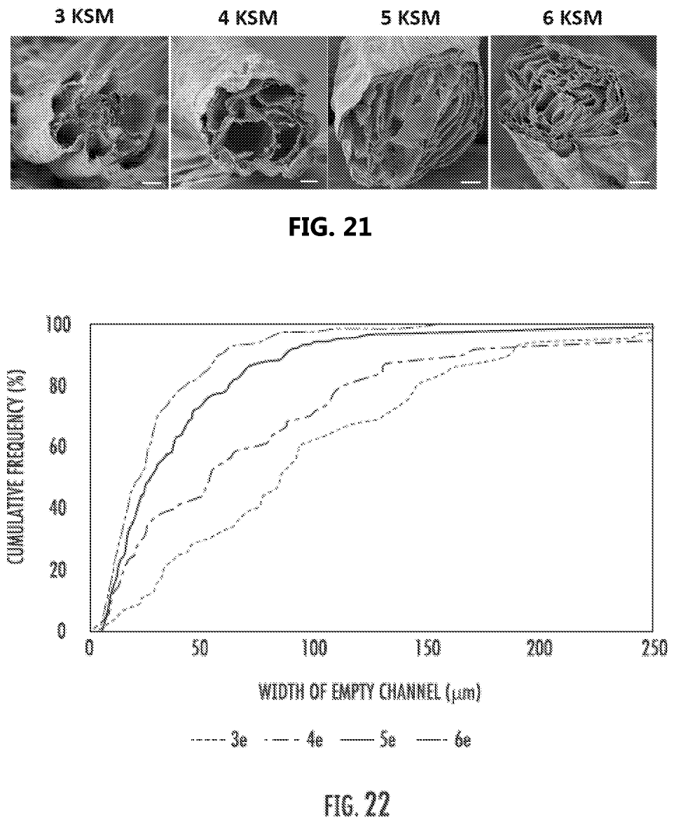

FIG. 21 shows SEM micrographs of fibers printed using 3, 4, 5, and 6 KSM elements. As shown in FIG. 21, the resulting fibers exhibited internal lamellar structures. The lamellar walls exhibited an average thickness of approximately 840 nm. Average channel widths decreased (and lamellar wall surface area increased) as the number of KSM elements increased. FIG. 22 is a plot showing the distribution of channel widths in high-surface/volume, perfusable microstructures formed using a KSM having 3, 4, 5, and 6 KSM elements. The channel width results are summarized in the table below.

| | Width (μm) | |
|---|---|---|
| KSM elements | W₅₀ | W₉₀ |
| 3 | 85 | 187 |
| 4 | 53 | 166 |
| 5 | 28 | 87 |
| 6 | 22 | 59 |

### Example 4. Bioreactors Employing High Surface/Volume and Perfusable Microstructured Scaffolds Prepared by Chaotic Printing

Expansion of progenitor cells while maintaining their stemness, or differentiating expanded cells, requires dense 3D culture environments of which few have been tested. Most that have been tested are assemblies of beads or sheets (lamina) inside a perfusion system. Nature generates densely packed micro- and nano-laminar structures to enable key functionalities within cells, tissues, and organs. Due to fabrication limitations in resolution and speed, prior to the proposed use of chaotic lamina it is not been possible to create micro- or nano-scale lamina cell culture systems like those found in nature. However, the proposed micro- or nano-laminar systems would offer extensive amounts of surface area per unit volume in small footprint systems.

In this Example, we propose chaotic 3D lamina printing—the use of chaotic flows—for the rapid generation of complex, high-resolution microstructures as the basis for highly modular and scalable bioreactor devices for single and multi-cell expansion and differentiation. In the Examples above, we demonstrated that simple and deterministic chaotic flow induced in a viscous liquid, through its repeated stretching and folding action, can deform a bioink (i.e., a drop of a miscible liquid, fluorescent bead, or cell) at an exponential rate to render a densely packed lamellar microstructure that is then preserved by curing or photocrosslinking. This exponentially fast creation of fine microstructures greatly exceeds the resolution and speed of currently available 3D printing techniques. Diverse applications for this technology can be envisioned, including the development of densely packed catalytic surfaces and highly complex multi-lamellar and multi-component tissue-like structures for biomedical applications. However, here we describe the use of these strategies to prepare scaffolds for use in a chaotic lamina bioreactor system.

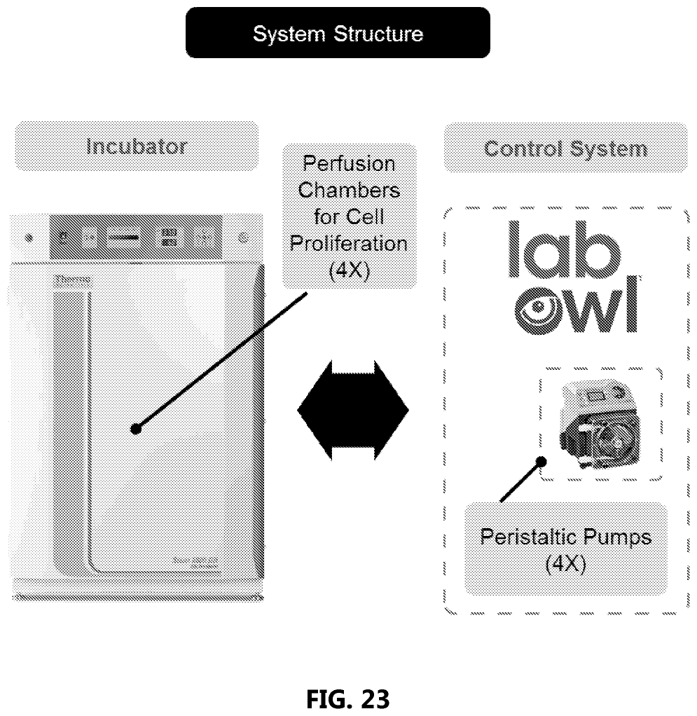

Closed bioreactor systems that permit the large-scale manufacture of cells and tissues. The proposed chaotic lamina bioreactor systems can provide for extremely high-density cell expansion in a modular bioreactor system. We are initially focusing on the culture of Bone Marrow-derived human Mesenchymal Stem Cells (BM-hMSCs), where current cell-based therapies require 10's to 100's of millions of cells per patient. Since BMhMSCs have to attach in order to survive and proliferate, the amount of surface area per liter becomes critical. Somewhat more surface area can be provided by coiling tissue culture plastic tubes (also referred to as “hollow fiber” systems) that can be perfused, such as in the Terumo BCT (Tokyo, Japan) system. The next generation in expanded surface area has been microbead or microcarrier systems referred to as Quantum Cell Expansion (QCE). These have proven risky as nutrient access to, and cell removal from, the inter-bead space is hazardous for cells at best. Advanced spinner flask, wave, and stirred tank systems, such as the Vertical Wheel8 (PBS Biotech, Camarillo Calif.), potentially allow for containers not restrained by incubator conditions. Stir tanks move away from the limited size of conventional incubator-based bioreactors. However, they make it nearly impossible to monitor microenvironment conditions, especially in regard to modeling and validating flow to all cells with local sensors. This makes it difficult to adjust and confirm universal environmental gases (FIG. 23), nutrient, and growth factor delivery, as well as waste removal. Our approach to solving these problems is to provide sensors near relatively compact, highly modular scaffolds for cell culture (e.g., chaotic lamina scaffolds, such as rods, discussed below), allowing for the adjustment of multiple channel parameters for each of a scalable series of chaotic laminae bioreactors.

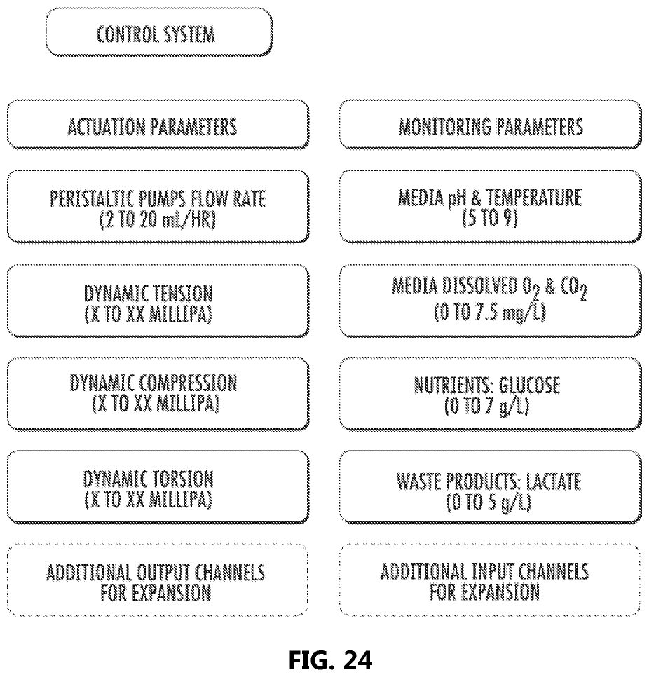

Bioreactors that enable precision control over medium composition and feedback monitoring of differentiation efficiency. There are primarily three activities of cells in bioreactors, scaffolds, or cell-based therapies that must be controlled: attachment, proliferation, and differentiation (maturation). These activities require both the proper substrate and media cues. These cues can be mechanical or involve cell signaling. The chaotic lamina bioreactor arrays cells on thin sheets or lamina in a way that can result in a very high number and density of cells. This can allow for cell attachment (to chaotic laminae) and proliferation (expansion) of BM-hMSCs without losing stemness. In initial implementations, the bioreactor can include sensors to monitor pH, temperature, dissolved CO₂, and O₂, glucose, and lactate (FIG. 24). In future implementations, other sensors (e.g., ammonia sensors) can be included as needed. If desired, circulating growth factors (cytokines), antibiotics, etc. can be introduced into the bioreactor (with sensors if desired to monitor levels of the circulating growth factors, antibiotics, etc.). A control system can utilize readings from the sensors and operate pumps to maintain desired environmental conditions within the bioreactor. In some embodiments, growth factors can be bioconjugated to the chaotic laminae scaffolds.

Bioreactors that automatically monitor, store, and replenish media. As density and perfusion forces increase, shear stress will be monitored. However, it is not expected to be a significant, much less an insurmountable, factor in chaotic laminae bioreactors. Our perfusion system will allow single, shared, and multiple media reservoirs. As the opening concentrations of nutrients, cytokines, antibiotics wane and waste product concentrations increase, our system will have the ability to adjust the rate of circulation of this media to control the concentration of the tracked growth factors until a new reservoir need be accessed, thereby allowing more until it is necessary to exchange old media for fresh.

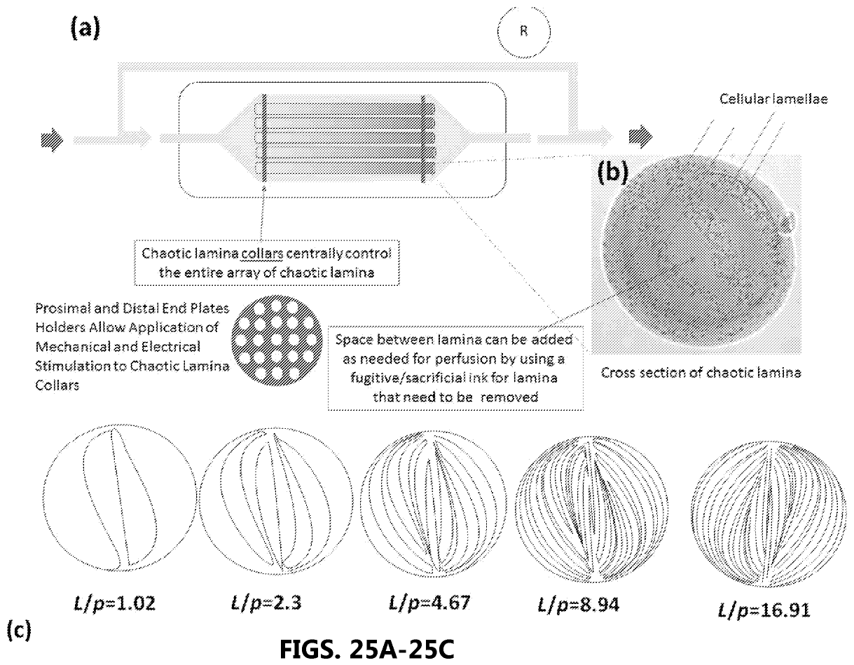

A multi-modal, large-scale bioreactor that can be used for multiple tissues and that can provide the necessary electrochemical and physiological stimuli to mature engineered tissues. Multimodal bioreactors have been attempted, with most using optical or acoustic sensors. However, these systems often fail to scale for the proliferation and differentiation of large numbers of cells because of difficult Computational Fluid Dynamic (CFD) and Fluid Structure Interface (FSI) modeling for large systems with complex geometries (e.g., inlet and perfusion chamber geometries that are difficult to model and validate) and inhomogeneous flow control (e.g., stir tank). Instead, in the bioreactors described herein, chaotic lamina scaffolds (e.g., formed as a rod, fiber, or bundle of fibers) equipped with collars can be arrayed in a cylindrical or application-specific geometry with a single input plate that can apply mechanical (e.g., tension, compression, and/or torsion) and/or electrical stimulation to the proximal and distal collars. See FIGS. 25A-25C.

A modular bioreactor system that can multiplex with existing technologies for fluid management and cell selection. Part of the bioreactor design involves the use of easily accessed media reservoirs. In regard to cell selection, this can be controlled at two points. The chaotic lamina system allows control over both points. First, in terms of cell seeding, the biofabrication (i.e., simultaneous 3D printing and cell seeding) methods described herein allow for the formation of adjacent lamina with a different types of cells (see Examples above). This sheet, or “layer”, differentiated system mimics structures in the developing embryo, fetus, and child, as well as a healing wound. The second point where cell selection is important is at cell harvest following proliferation or cell differentiation. Harvest can be accomplished by one of two means. First, the bioink can include a polymer that degrades or sufficiently weakens by the planned harvest point, such that a mild mechanical agitation removes cells from the scaffold. Alternatively, a mild enzyme that only targets the bioink (laminae) could be used.

With respect to “fluid management, in some implementations, the chaotic lamina rods can be flash frozen (e.g., for storage). If desired, the rods can be individually handled (e.g., by a robot that places the rods into storage where immediate use following cell expansion is not desirable. For example, if one was not ready to use the cells at the point they must be harvested to stop expansion, they may be stored (frozen).

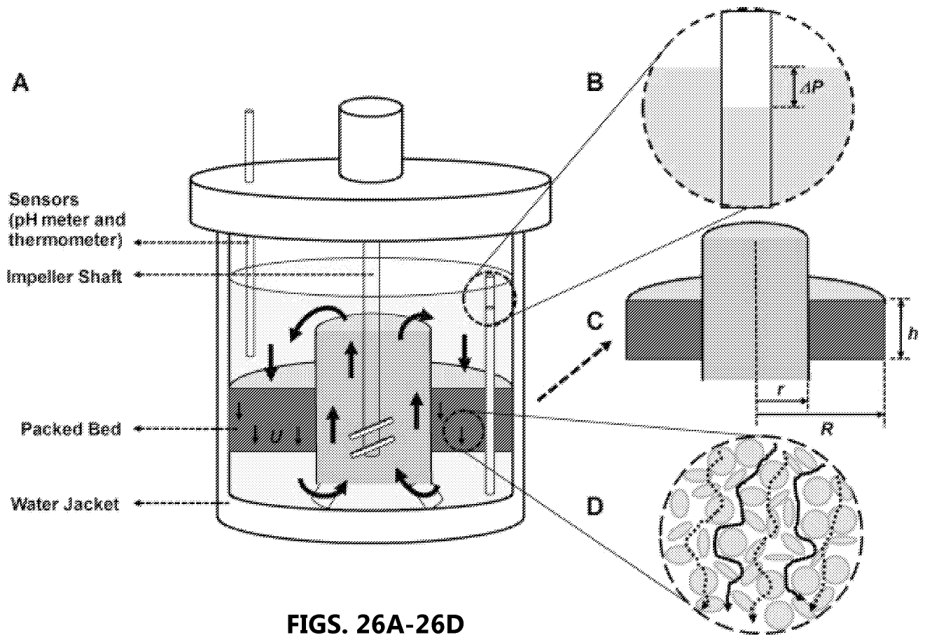

Bioreactors that maximize surface-area-to-volume ratios, control shear and other environmental parameters, and ensure maximum cell recovery during enzymatic release steps. The chaotic laminae described herein offer unprecedented surface-area-to-volume (SAV) ratios. The chaotic lamina is capable of producing SAV ratios that, until now, were only possible in living organisms. The termite hindgut, which requires anoxia for its activities, has been cited as one of the highest SAV ratio “bioreactors”, achieving 5000 m²/m³, or 5000 m-¹. Conventional stir bioreactors utilizing stacks of fibrous sheets provide 116.7 m⁻¹ SAV (FIG. 26). In the examples above, model chaotic lamina rods were produced having an SAV of 710 m⁻¹, with higher levels possible. Future rods can exhibit SAVs of 4600 m⁻¹ (FIGS. 25A-25C).

Studies will determine design-dependent aspects and the ability to modulate these elements to maintain desired cell expansion rates. High SAVs are known to make it simpler to modulate shear. This is seen as improved cell viability, which was observed to be improved 90-97% in the chaotic lamina systems above.

Summary

As described above, the proposed chaotic lamina-based bioreactors can function as incubator-based systems allowing large numbers of cells to be expanded in the smallest possible space. Rather than state of the art indirectly tracked stirring systems, the chaotic laminar rods will have highly accurate sensors tracking environmental gases, nutrient, growth factor delivery, and waste removal. Unlike indirect testing in current systems, the chaotic lamina sensors will determine both when new media needs to be added, alert the user by the internet, and will automatically control media flow rates of available media to ensure cell expansion rates. Unlike non-existent commercial and small scale, home-made systems that deliver non-homogenous mechanical or electrical stimulation, the collared chaotic laminar rod system will allow apply mechanical and electrical stimulation as well as allow automation of cell harvest and storage (freezing). Chaotic lamina rods will have SAV that is more than six times greater than current commercial systems. Once the prototype chaotic lamina system is validated, it is expected that there is potential to increase cell yield by roughly 4 fold in a fraction of the space. Current lab-based use of non-GMP cells in standard footprint incubators can expand about 50 flasks of 1 million to 100 million cells (i.e., 5 billion cells). Whole room systems are available to expand up to 25-30 billion cells. The bioreactors described herein will accomplish this in an incubator. Moreover, chaotic lamina rod modularity will improve expansion speed, require less handling or space for freezing, and the use of standard incubators with direct sensing/controls will reduce cost at least 10×.

The chaotic lamina platform will provide a single or multi-cell type expansion bioreactor with an SAV of at least 700 m-1 that has automated, direct chamber outlet tracking of media, flow actuation, and remote notification of the need for media additions. The platform will be validated for four independent, separately tracked and actuated, bioreactor lines. Each line will be capable of applying independent mechanical and electrical stimuli and will be able to provide for robotic removal of chaotic lamina rods. The platform will include manual and robotic cell isolation from chaotic lamina rods for immediate use and rod-based storage (freezing). The system will be contained in a small footprint incubator that facilitates automated and highly accurate control of environmental gases, humidity, and temperature.

The compositions, systems, and methods of the appended claims are not limited in scope by the specific compositions, systems, and methods described herein, which are intended as illustrations of a few aspects of the claims. Any compositions, systems, and methods that are functionally equivalent are intended to fall within the scope of the claims. Various modifications of the compositions, systems, and methods in addition to those shown and described herein are intended to fall within the scope of the appended claims. Further, while only certain representative compositions, systems, and method steps disclosed herein are specifically described, other combinations of the compositions, systems, and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than where noted, all numbers expressing geometries, dimensions, and so forth used in the specification and claims are to be understood at the very least, and not as an attempt to limit the application of the doctrine of equivalents to the scope of the claims, to be construed in light of the number of significant digits and ordinary rounding approaches.

Unless defined otherwise, all technical and scientific terms used herein have the same meanings as commonly understood by one of skill in the art to which the disclosed invention belongs. Publications cited herein and the materials for which they are cited are specifically incorporated by reference.

What is claimed is:

1. A method for the preparation of a perfusable scaffold for cell culture, the method comprising:
providing a bioink composition and a fugitive ink composition;
chaotic printing the bioink composition and the fugitive ink composition to generate a microstructured precursor comprising a plurality of lamellar structures formed from the bioink composition;
curing the bioink composition to form a cured scaffold precursor; and
removing the fugitive ink from the cured scaffold precursor, thereby forming the perfusable scaffold.

2. The method of claim 1, wherein the method further comprises dispersing a population of cells in the bioink composition prior to the chaotic printing.

3. The method of claim 1, wherein the method further comprises seeding the perfusable scaffold with a population of cells.

4. The method of any of claims 2-3, wherein the cells comprise pluripotent stem cells, multipotent stem cells, progenitor cells, terminally differentiated cells, endothelial cells, endothelial progenitor cells, immortalized cell lines, primary cells, or any combination thereof.

5. The method of any of claim 1-4, wherein chaotic printing of the bioink composition and the fugitive ink composition comprises inducing laminar flow of the bioink composition and the fugitive ink composition through a mixer that chaotically mixes the bioink composition and the fugitive ink composition to form lamellar interfaces between the bioink composition and the fugitive ink composition.

6. The method of any of claims 1-5, wherein chaotic printing of the bioink composition and the fugitive ink composition comprises coextruding the bioink composition and the fugitive ink composition through a mixer that chaotically mixes the bioink composition and the fugitive ink composition to form lamellar interfaces between the bioink composition and the fugitive ink composition.

7. The method of any of claims 5-6, wherein the mixer comprises a static mixer, such as a Kenics static mixer.

8. The method of any of claims 1-7, wherein the perfusable scaffold an average striation thickness of from 10 nm to 500 μm.

9. The method of any of claims 1-8, wherein the perfusable scaffold exhibits a surface-area-to-volume (SAV) of from 400 m⁻¹ to 5000 m⁻¹.

10. The method of any of claims 1-9, wherein the perfusable scaffold exhibits a surface density of at least 0.05 m² cm⁻³.

11. The method of any of claims 1-10, wherein the perfusable scaffold is produced in the form of a fiber.

12. The method of any of claims 1-11, further chaotic printing the bioink composition and the fugitive ink composition comprises 3D printing, electrospinning, extrusion, or any combination thereof.

13. The method of any of claims 1-12, wherein the bioink composition comprises a polymer.

14. The method of claim 13, wherein the polymer comprises a hydrogel-forming agent.

15. The method of any of claims 13-14, wherein the polymer comprises a polysaccharide, such as alginate, hyaluronic acid, agarose, or any combination thereof.

16. The method of any of claims 13-15, wherein the polymer comprises a protein or peptide, such as gelatin, collagen, or any combination thereof.

17. The method of any of claims 13-16, wherein the polymer comprises a synthetic polymer, such as a polyester (e.g., poly(propylene fumarate) (PPF), polycaprolactone, poly(lactic-co-glycolic acid), polylactic acid, polyglycolic acid, or any combination thereof).

18. The method of any of claims 13-17, wherein the polymer is crosslinkable.

19. The method of any of claims 13-18, wherein the polymer is present in an amount of from 0.5% to 20% by weight, based on the total weight of the bioink composition

20. The method of any of claims 1-19, wherein the bioink composition comprises a bioactive agent, such as a growth factor, growth inhibitor, cytokine, steroid, antibiotic, morphogen, or any combination thereof.

21. The method of claim 20, wherein the bioink composition comprises a polymer and wherein the bioactive agent is conjugated to the polymer.

22. The method of claim 20, wherein the bioink composition comprises a population of nanoparticles, a population of microparticles, or any combination thereof, and wherein the bioactive agent is conjugated to the particles.

23. The method of claim 20, wherein the bioink composition comprises a population of nanoparticles, a population of microparticles, or any combination thereof, and wherein the bioactive agent is encapsulated or dispersed in the particles.

24. The method of any of claims 1-23, wherein the fugitive ink composition comprises a polymer.

25. The method of claim 24, wherein the polymer comprises a poly(alkylene oxide) block copolymer, such as a polyoxyethylene-polyoxypropylene (PEO-PPO) block copolymers (e.g., a poloxamer).

26. The method of any of claims 24-25, wherein the polymer is present in an amount of from 0.5% to 20% by weight, based on the total weight of the fugitive ink composition.

27. A perfusable scaffold for cell culture prepared by the method of any of claims 1-26.

28. A bioreactor comprising
a plurality of perfusable scaffolds, each prepared by the method of any of claims 1-26.

29. The bioreactor of claim 28, wherein each of the plurality of the perfusable scaffolds is in the form of a rod, fiber, or bundle of fibers.

30. The bioreactor of any of claims 28-29, further comprising a housing enclosing the plurality of perfusable scaffolds.

31. The bioreactor or any of claims 28-30, wherein each of the plurality of perfusable scaffolds is operatively coupled to a proximal collar and a distal collar.

32. The bioreactor of claim 31, wherein the bioreactor further comprises a first single input plate operatively coupled to each of the proximal collars, and a second single input plate operatively coupled to each of the distal collars.

33. The bioreactor of claim 32, wherein the first single input plate and the second single input plate are configured to apply mechanical stimulation to the plurality of perfusable scaffolds.

34. The bioreactor of any of claims 32-33, wherein the first single input plate and the second single input plate are configured to apply electrical stimulation to the plurality of perfusable scaffolds.

35. The bioreactor of any of claims 28-34, wherein the bioreactor further comprises a pH monitoring and control system, a temperature monitoring and control system, an O₂ monitoring and control system, a CO₂ monitoring and control system, a glucose monitoring and control system, a lactate monitoring and control system, a fluid flow monitoring and control system, or any combination thereof.
